(19) United States
(12) Patent Application Publication
(10) Pub. No.: US 2013/0274233 A1
(43) Pub. Date: Oct. 17, 2013

(54) MODULATORS OF HEDGEHOG SIGNALING PATHWAY

(71) Applicant: PRESIDENT AND FELLOW OF HARVARD COLLEGE, (US)

(72) Inventors: Yu WANG, Madison, WI (US); Andrew P. MCMAHON, Pasadena, CA (US); Rubin L. LEE, Wellesley, MA (US)

(21) Appl. No.: 13/855,930

(22) Filed: Apr. 3, 2013

Related U.S. Application Data
(60) Provisional application No. 61/619,649, filed on Apr. 3, 2012.

(51) Int. Cl. A61K 31/166; A61K 31/58

(52) U.S. Cl. CPC A61K 31/166; A61K 31/58
USPC 514/174; 514/615; 435/375

(57) ABSTRACT

The invention provides compounds and methods for modulating the Hedgehog signaling pathway. The compounds modulate the translocation and/or accumulation of smoothened to the primary cilia.

### RELATED APPLICATIONS

This application claims benefit under 35 U.S.C. §119(e) of the U.S. Provisional Application No. 61/619,649, filed Apr. 3, 2012, the content of which is incorporated herein by reference in its entirety.

### GOVERNMENT SUPPORT

This invention was made with government support under grant no. R37 NSO33642 awarded by the National Institutes of Health. The government has certain rights in the invention.

### TECHNICAL FILED

The invention relates to compositions and methods for modulating the Hedgehog signaling pathway.

### BACKGROUND

Hedgehog (Hh) signaling plays an essential role in developmental processes and adult tissue homeostasis¹. An increasing body of evidence identifies the Hh pathway as a contributing factor in the growth of a variety of human cancers. The loss of normal regulatory control of the Hh pathway within a subset of Hh responsive cells leads directly to the initiation of particular solid tumors, notably basal cell carcinoma (BCC), the most prevalent cancer in the Caucasian population², and medulloblastoma (MB), the most common childhood brain cancer³. In other cancers, Hh signals from tumor cells appear to condition the local environment to favor tumor growth. This category includes a broad spectrum of high incidence cancers, particularly those in breast, lung, liver, stomach, pancreas, prostate, and gastro-intestinal tract⁴⁻⁵. The potential of Hh targeted cancer therapy has stimulated an extensive search for Hh pathway antagonists. Typically, drug discovery screens have broadly sampled the Hh pathway looking for agents capable of silencing a Hh signal-dependent transcriptional response. Although small-molecule “hits” may occur at any point in the pathway that ultimately translates to an altered transcriptional response, Smoothened (Smo), has emerged as the prevalent target,⁶⁻⁷ Smo is essential for all pathway activity, and activating mutations in Smo have been observed in both human BCC and MB. Smo antagonists have entered clinical trials⁸, and successful repression of tumorigenesis in patients with invasive or metastatic forms of BCC has validated the concept of Hh targeted cancer therapy⁹.

An obligatory step in the activation of Hh signaling is the accumulation of Smo in the primary cilium (PC), a tubulin-scaffolded membrane extension templated by the centriole (FIG. 1a). While all small molecule Smo agonists examined so far induce Smo accumulation in the PC, various Smo antagonists affect Smo localization in distinct ways (FIG. 1a)¹⁰⁻¹². SANT-1 and SANT-2 inhibit both Hh pathway activation and Sonic Hedgehog (Shh) induced Smo accumulation within the PC¹⁰⁻¹². The actions of GDC0449 (also known as RG3616), a Smo antagonist in clinical trials, have not been reported^{9,13-14}. In contrast, the inventors, and others, have reported that Cyclopamine (Cyc), a natural product of the plant Veratrum californicum that binds Smo and inhibits pathway activation, behaves as a pseudo-agonist promoting Smo accumulation within the PC¹⁰⁻¹². Further, forskolin (FKL), a putative protein kinase A (PKA) activator, also inhibits Hh pathway and promotes Smo ciliary accumulation though indirectly through PKA stimuylation¹⁰.

Although both categories of inhibitors can be potentially useful in targeted cancer therapy, an understanding of the differential outcomes on Smo ciliary translocation is important in viewing their actions. Thus, there is a need in the art for broad spectrum of small molecules that inhibit Hh signaling without conferring the risk of hyperactivity.

### SUMMARY

In one aspect, the invention provides a method of modulating Hedgehog signaling pathway, the method comprising contacting a cell with a compound described herein.

In another aspect the invention provides a method of modulating differentiated state, survival, and/or proliferation of a cell, the method comprising contacting the cell with a compound described herein.

In yet another aspect, the invention provides a method of treating a subject, the method comprising administration of a therapeutically effective amount of the compound to subject, wherein the subject is in need of modulating Hedgehog signaling pathway.

In still yet another aspect, the invention provides a method of modulating cellular sensitivity to Hedgehog signaling, the method comprising contacting a cell with a compound described herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1C show a high content Smo antagonist screen-image analysis and assay validation. (a) A field of cells in a typical well. The cell number was calculated by counting Hoechst stained nuclei. The PC were precisely segmented as Ivs::tagRFPT positive structures and hSmo::EGFP intensity was quantified in the PC. (b) Representative images of the dose-dependent inhibition of Smo::EGFP ciliary accumulation by GDC0449. The concentrations of GDC0449 used to obtain these images were 0, 0.15 nM, 1.3 nM, 12 nM, 111 nM, 1 μM from left to right. Scale bar: 10 μm. (c) Key measurements from high content image analyses. The cell number was determined by counting Hoechst stained nuclei. Ivs::tagRFPT positive structures were precisely segmented as the PC and Smo::EGFP intensity within the PC was quantified. The Ivs+ cilium count and Smo+ cilium count were determined based on arbitrary thresholds; the mean (±S.D) shown is based on four replicates.

FIGS. 2A-2D show identification of compounds disrupting the PC. (a-b) HPI-4, an inhibitor of ciliogenesis, was identified in the assay. (c-d) Vinblastine (VBN), which disrupts microtubules, and leads to disruption of the PC, was also identified through general effects on Ivs::tagRFPt; the mean (±S.D) for the Smo localization assay and Gli-luciferase transcriptional reporter assays was calculated from four replicates (a and c). HPI-4 and VBN were used at 50 μM and 370 nM, respectively, to generate the representative images in (b) and (d). Scale bar: 10 μm.

FIGS. 3A-3I show DY131 displays a conserved mechanism for Smo inhibition similar with previously identified antagonists. (a) Structure of DY131 and GSK4716. (b-c) Representative images (b) and quantification (c) of DY131 and GSK4716 inhibition of Hh induced Smo accumulation at the primary cilium. 500 nM SANT-1 was used as a positive control for pronounced inhibition. DY131 and GSK4716 were used at 3.75 μM and 7.5 μM, respectively, for data in (b). Scale bar: 5 μm. (d) Gli-luciferase measurements indicate dose-dependent inhibition of Hh pathway activity by both DY131 and GSK4716. Data show the means (±S.D.) from quadruplicate samples. Image analysis was based on over 300 cells per sample. (e) Representative images showing Smo::EGFP and SmoM2::EGFP overexpressing cells treated with vehicle or 1.1 μM DY131. Scale bar: 5 μm. (f) Image analysis of quadruplicate samples, plotting mean (±S.D.) of over 300 cells analyzed in each sample. (g) Dose-response curves displaying DY131 inhibition of wild-type Smo, and SmoM2 activity. Data show mean (±S.D.) in quadruplicate samples. Representative images (h) and quantification (i) of Bodipy-Cyc competition experiments. Cyc, SANT-1, and DY131 were each used at 1.1 μM in (h). Scale bar: 10 μm. Data show the mean (±S.D.) in quadruplicate samples (i), analyzing 50-100 transfected cells in each sample.

FIGS. 4A-4K show SMANT displays an unprecedented mechanism for Smo inhibition. (a) Structure of SMANT and SMANT-2. (b-c) Representative images (b) and quantification (c) of SMANT and SMANT-2 inhibition of Hh induced Smo accumulation at the primary cilium. 1 μM SANT-2 was used as a positive control. SMANT and SMANT-2 were used at 7.5 μM for data in (b). Scale bar: 5 μm. (d) Representative images showing Smo::EGFP and SmoM2::EGFP overexpressing cells treated with vehicle or SMANT. SMANT was applied to wild-type Smo and SmoM2 expressing cells at 7.5 μM and 30 μM respectively. Scale bar: 5 μm. (e) Image analysis of quadruplicate samples shown in (d), plotting mean (±S.D.) of over 300 cells analyzed in each sample. (f-g) Representative images (f) and quantifications of Smo ciliary localization (g) showing Smo::EGFP/Ivs::tagRFPT cells treated with 100 nM SAG combined with vehicle, SANT-1, GDC0449, or SMANT. SANT-1, GDC0449 and SMANT were used at 104, 120 nM, and 60 μM. (h) Gli-luciferase measurement of dose-dependent inhibition of Hh pathway activity by SMANT upon Shh stimulation, overexpression of Smo and SmoM2 respectively, or treatment with 0.2 μM or 1 μM of SAG. Data show the means (±S.D.) from triplicate samples. (i) Gli-luciferase measurements in suFU−/− mouse embryonic fibroblasts treated with DY131 and SMANT respectively. GDC0449 and GANT61 were used as negative and positive controls respectively. (j-k) Representative images (j) and quantification (k) of Bodipy-Cyc competition experiments for SMANT. SANT-1 served as a control for competition activity. SMANT was used at 30 μM in (h). Scale bar: 5 μm. Data show the mean (±S.D.) from quadruplicate samples (i), analyzing 100-200 transfected cells in each sample.

FIGS. 5A-5E show DY131 and SMANT inhibit proliferation of cerebellar granule-cell neural progenitors (CGNP) without conferring hypersensitivity to Shh stimulation. (a-b) representative images (a) and quantification of phospho-histone H3(pH3) positive cells (b) upon co-treatment with 0.625 μM DY131 or SMANT with Shh ligand. (b) P<=0.001 in t-test for all samples treated with DY131 or SMANT at 0.625 μM and above compared with DMSO treated controls. (c-e) In contrast to Cyc, GDC0449, DY131, and SMANT do not confer prolonged hypersensitivity to Shh stimulation in either Gli responsive reporter (c) or CGNP proliferation assays (d-e). Hh signaling activity and CGNP proliferation were measured after treatment with vehicle, Cyc (5 μM), GDC0449 (500 nM), DY131 (10 μM), or SMANT (10 μM) separately. Samples were analyzed in quadruplicate; data show the mean (±S.D.). For the Gli-luciferase reporter assay (c), cells stimulated by Shh for a relatively short time period (12 hours) displayed a modest but significant inductive response (#p<0.003 in at test comparing to a DMSO primed 0 nM Shh treatment). The response was enhanced by pre-treating cells with Cyc (##P<0.003 in t test comparing toDMSO priming and stimulation with the same concentration of Shh) whereas pretreatment with GDC0449, DY131, or SMANT showed no enhancing activity (P>0.05 in t test comparing samples primed with DMSO and stimulated with the same concentration of Shh). For the CGNP assay (d-e), cells were treated for 3 days. +p<0.0001 in at test comparing the effects of DMSO (control) or any of the antagonists of Smo ciliary accumulation.

FIGS. 6A-6C show differential modulation of Smo cilial translocation and its signaling by several antagonists. (a) A schematic showing different Smo ciliary translocation behaviors induced by different Hh antagonists. Although all inhibited Smo activity, SANT-1, SANT-2, and GDC0449 inhibited Smo ciliary accumulation, whereas the direct Smo antagonist Cyc and FKL, a PKA activator, that indirectly affects pathway activity, both promote Smo accumulation to the PC. (b-c) Treatment with Hh antagonists (Cyc: 5 μM; FKL: 100 μM; SANT-1: 500 nM; GDC0449: 500 nM) modifies Smo cilial localization (b) and inhibit Shh-stimulated pathway activity (P<1E-4 in t test) (c). SAG was included as a control agonist that has been shown to bind Smo and promote ligand independent Smo accumulation on the PC. Data show the mean from quadruplicate treatments (±the standard deviation, S.D.).

FIG. 7 shows the workflow for a Smoothened cell-based screen monitoring accumulation at the primary cilium. Small molecule libraries were applied to Smo::EGFP/Ivs::tagRFPT cells. Potential antagonists of Smo cilial accumulation were identified in an assay based on movement of Smo::EGFP.

FIGS. 8A and 8B show validation of high content Smo antagonist screen with SANT-1. (a) Representative images of the dose-dependent inhibition of Smo::EGFP cilial accumulation by SANT-1. The concentrations of SANT-1 used to obtain these images were 0, 1.5 nM, 14 nM, 123 nM, 1111 nM from left to right. Scale bar: 5 μm. (b) Key measurements from high content image analyses. The mean (±S.D) shown is based on four replicates.

FIG. 9 shows plots of relative Smo::EGFP+ cilium count normalized upon Shh+DMSO treated samples for putative Smo antagonist screen. Each dot represents the measurement of over one thousand cells in each well. Compound libraries were assayed at 10 μM for this plot. SANT-1 and SAG were used at 1 μM and Cyc was used at 10 μM.

FIGS. 10A-10E show indentification of AntagVIII. (a) structure. (b-d) AntagVIII inhibited both Smo cilial accumulation (b and c) and Hh pathway activity (d). Each treatment was analyzed in quadruplicate, measuring over 300 cells for each sample. AntagVIII was used at 1.875 μM for the representative image in (b). Scale bar: 5 μm (e) high content analyses on AntagVIII's dose dependent inhibition of Smo localization on the PC.

FIGS. 11A-11E show identification of AY9944. (a) Structure. (b-d) AY9944 inhibited both Smo cilial accumulation (b and c) and the Hh pathway activity (d). Each treatment was analyzed in quadruplicate, measuring over 300 cells for each sample. AY9944 was used at 10 μM for the representative image in (b). Scale bar: 5 μm (e) high content analyses on AY9944's dose dependent inhibition of Smo localization on the PC.

FIGS. 12A-12E show identification of Itraconazole (ICZ) and Ketoconazole (KCZ). (a) Structures. (b-d) ICZ and KCZ inhibited both Smo cilial accumulation (b and c) and Hh pathway activity (d). Each treatment was analyzed in quadruplicate, measuring over 300 cells for each sample. ICZ and KCZ were used at 3.3 μM and 10 μM for representative images (b). Scale bar: 10 μm (e) high content analysis of the dose dependent inhibition of Smo accumulation in the PC on ICZ or KCZ treatment.

FIGS. 13A and 13B show high content analyses indicate specific inhibition of Smo cilial localization by DY131 and GSK4716. High content measurements are displayed for the cell count, Ivs::tagRFPT positive cilial count, cilial Ivs::tagRFPT intensity and Smo::EGFP intensity in the PC. Samples were analyzed in quadruplicate images comprising 50-100 transfected cells. Data are plotted as the mean (±S.D.).

FIG. 14 shows DY131 does not inhibit Wnt signaling activity. Wnt signaling activity was measured in a Top-flash reporter assay (Corbit et al., Nature Cell Biology, 2008, 10, 70-76). A gradient of DY131 was applied to the cells with (Fold plotted) or without Wnt3a ligand (Control % plotted) conditioned medium. Means (±S.D.) from quadruplicate treatments are shown.

FIGS. 15A-15C show several ERR/ER ligands including tamoxifen, 4-hydroxytamoxifen (4-OHT), diethylstilbestrol, and hexestrol do not alter Smo cilial localization in presence or absence of Hh ligand. (a) Structures of ERR/ER ligands. (b) Mean (±S.D.) of relative Smo::EGFP positive cilium count after treating cells with various doses of ERR/ER ligands in the absence (Fold plotted) or presence (Control % plotted) of Hh ligand. Each treatment was analyzed in quadruplicate, examining over 600 cells per sample. (c) Representative images. All compounds were used at a concentration of 15 μM. Scale bar: 5 μm.

FIGS. 16A-16D show accumulation of SmoM2 in the PC is refractory to SANT-1 or GDC0449 inhibition. Representative images (a and c) and quantification (b and d) of the cilial intensity of Smo::EGFP and SmoM2::EGFP. SANT-1 and GDC0449 were used at 1.1 μM for the representative images; quadruplicate samples were analyzed and data show the means (±S.D.) of the EGFP intensity, examining over 300 cells in each treatment. Scale bar: 5 μm.

FIGS. 17A-17C show SmoM2 mutation confers resistance to inhibition by Cyc, SANT-1, or GDC0449. Hh pathway activity was induced by Hh ligand, over-expressing Smo or SmoM2. Dose dependent inhibition of Hh/Smo/SmoM2 signaling by Cyc, SANT-1, or GDC0449 was measured in quadruplicate samples. Data show the mean (±S.D.).

FIGS. 18A-18C show DY131 inhibits SAG induced Smo cilial accumulation and signaling. Representative images (a) and image quantification (b) showing the mean (±S.D.) determined in quadruplicate samples, examining over 300 cells per sample. Scale bar: 5 μm. Mean (±S.D.) of Gli luciferase activity measured in quadruplicated samples (c).

FIGS. 19A and 19B show high content analyses indicate specific inhibition of Smo cilial localization by SMANT and SMANT-2. High content measurements are displayed for the cell count, Ivs::tagRFPT positive cilium count, cilial Ivs::tagRFPT intensity and Smo::EGFP intensity in the PC. Samples were analyzed in quadruplicate images comprising 50-100 transfected cells. Data are plotted as the mean (±S.D.).

FIG. 20 shows SMANT does not activate nor profoundly inhibit Wnt signaling activity. Wnt signaling activity was measured in a Top-flash reporter assay¹. A gradient of SMANT was applied to the cells with (Fold plotted) or without Wnt3a ligand (Control % plotted) conditioned medium. Means (±S.D.) from quadruplicate treatments are shown.

FIGS. 21A and 21B show SMANT does not inhibit Cyc induced Smo cilial accumulation, whereas DY131, SANT-1, and GDC0449 effectively compete with Cyc. (a) representative images of cells treated with 5 μM Cyc and DMSO, GDC0449 (7.5 μM), SANT-1 (7.5 μM), DY131 (7.5 μM), or SMANT (15 μM) respectively. (b) image quantification showing the mean (±S.D.) determined in quadruplicate samples, examining over 300 cells per sample. Scale bar: 5 μm.

FIGS. 22A-22E show the effects of distinct Smo antagonists on Smo localization and Hh pathway activity. (a) Measurement of Hh signaling in Gli transcription reporter assays following treatment with either vehicle or Hh antagonists: Cyc (5 μM), FKL (100 μM), SANT-1 (500 nM), and GDC0449 (500 nM). Samples were analyzed in quadruplicate; mean is shown ±S.D. Pathway stimulation with Sonic hedgehog (Shh) was shorter (12 hrs) than in routine assays but a significant response was observed in this short time frame (#p<0.04 and ##p<1E-4 in t test). * indicates statistically significant hyperactivity in at test comparing pre-treated with vehicle treated cells (*p<0.02; **p<0.006 in t test). SANT-1 and GDC0449 pre-treated cells showed either a significant but lower induction (+p<0.05 in t test), or no reduction in activity (P>0.1 in t-test for the rest). (b-e) Representative images of Smo::EGFP in the PC (b and c) and quantified Smo::EGFP ciliary intensity (d and e); a slow turnover of Smo::EGFP was observed following Cyc (5 μM) and FKL (100 μM) withdrawal. No measurable change was observed in Ivs::tagRFPT, an independent primary cilium marker, over the same time period. All the Ivs::tagRFPT images in this report were shifted leftwards by 5 pixels to clearly show both Smo::EGFP and Ivs::tagRFPT signals. Experiments were conducted in quadruplicate, analyzing over 300 cells in each sample. Fluorescent intensity was plotted as the mean (±S.D.). Scale bar: 5 μm.

FIG. 23 shows cellular sensitivity to SAG stimulation after priming with various Hh antagonists. Hh signaling activity was measured in a Gli transcription reporter assay after pre-treatment with vehicle or various Hh antagonists including Cyc (5 μM), FKL (100 μM), SANT-1 (500 nM), and GDC0449 (500 nM). Samples were assayed in quadruplicate and means (±S.D.) were displayed. A short 12 hr stimulation with SAG induced a weak but significant response (#p<0.0002 in t test). * indicates statistical significant difference in t test when compared with cells treated with vehicle (*p<0.03; **p<0.005 in t test). In contrast, SANT-1 and GDC0449 treated cells showed a significantly lower induction (+p<0.05 in t test) or no statistical difference (p>0.05 in t-test for remaining samples) when compared with cells treated with vehicle.

FIGS. 24-29 show an investigation of structure-activity relationships (SAR) for DY131 and its analogs.

FIGS. 30-36 show an investigation of structure-activity relationship analysis of inhibitory activity od Smo Cilila accumulation by SMANT and its analogs. FIG. 30 shows classes of Azacyclo-N-alkyanidies assayed in Smo antagonist high content screen.

FIGS. 37A-37D show glucocorticoids induce Smo accumulation at the primary cilium. (A) Structures of 10 representative naturally occurring and synthetic GCs (names in bold). (B) Related dose response curves for accumulation of Smo in the PC using the subset of GCs indicated in (A). The mean (±S.D.) was calculated for four replicates analyzing several hundred cells in each sample. (C) Key positions correlating with Smo accumulation activity in the GC scaffold are highlighted in red. (D) Representative images of dose-dependent accumulation of Smo at the PC in response to stimulation by a synthetic GC, fluocinolone acetonide (FA). Scale bar: 10 μm.

FIGS. 38A-38H show FA sensitizes cells to Hh stimulation and competes with Cyc binding to Smo (A) Modest but significant activation of the Hh pathway at high doses of FA, measured by a Gli responsive luciferase reporter activity in NIH/3T3 cells. FA action is blocked by Smo antagonists SANT-1 and Cyc. * P<0.0001 (t-test), comparing with DMSO, FA+SANT-1, or FA+Cyc. (B) FA up-regulates the expression of Hh target genes Ptch1 and Gli1 in NIH/3T3 cells. #P<0.02 (t-test) (C-D) Measurement of Hh pathway activity in cells treated simultaneously with a fixed concentration of FA and different concentrations of Shh ligand (C), or with a fixed concentration of Shh ligand, and different concentrations of FA (D). Treatment with Shh ligand and DMSO were used for comparison. (E) Measurement of Hh pathway activity after stimulation with various concentrations of Shh ligand following priming treatments with 10 μM FA (red curve). DMSO prime treatments (blue curve) were used for comparison. (F) Measurement of Hh pathway activity after stimulation with a fixed dose of Shh ligand following priming treatments with different concentrations of FA (red curve) or when pathway activity was stimulated by expressing a constitutively active SmoM2 variant (blue curve). All Gli-luciferase assay samples were replicated four times. The qRT-PCR in (B) was performed in triplicate. Data represent mean (±S.D.). (G) Schematics showing the structures of Bodipy-Cyc and Smo. Critical sites in the 6^{th} and 7^{th} transmembrane domains of Smo, that are likely important for direct interactions are highlighted by rectangles. (H) Representative merged images from Bodipy-Cyc competition assays. Transfected cells can be identified by co-labeling with nuclear localized tagRFPT. (I) Quantification of Bodipy-Cyc fluorescence signal: each data point represents values from 50-100 transfected cells. The controls including those from a parental plasmid (pCIT), Smo and SmoM2 expressing cells were displayed as the dashed lines. Mean (±S.D.) was calculated from four replicate samples. Scale bar: 10 μm.

FIGS. 39A-39C show drug-drug interaction between FA and GDC0449. (A) GDC0449 dose-dependent inhibition of Shh stimulated Hh pathway activity in the presence or absence of 10 μM FA, or SmoM2 expressing cell lines. (B) Representative images of Smo::EGFP/Ivs::tagRFPT cells treated with GDC0449 and Shh in the presence or absence of 10 μM FA. GDC0449 was co-applied at 111 nM and 1,111 nM respectively with Shh and Shh+FA. (C) Relative Smo::EGFP+ cilium count of GDC0449's dose-dependent inhibition of Shh-ligand stimulated accumulation of Smo in the PC in the presence or absence of 10 μM FA. Measurements were performed in quadruplicate. Several hundred cells were analyzed in each sample to assess the accumulation of Smo in the PC from data in (B). Data plotted are mean (±S.D.). Scale bar: 5 μm.

FIG. 40A-40F shows FA modulates proliferation of cerebellar granule-cell neural progenitors (CGNP). CGNP proliferation was quantified based on percentage of pH3 positive cells. Representative images and measurements were shown for FA dose dependent modulation of CGNP proliferation (A-B), promotion of Shh stimulated NP proliferation by 10 μM FA(C-D), and GDC0449 dose-dependent inhibition of Shh stimulated CGNP proliferation in the presence or absence of 10 μM FA(E-F). GDC0449 was applied at 100 nM in (E). Mean (±S.D.) was calculated from four replicate samples each containing over a thousand cells. *p=0.0003(t-test). #p<0.0001(t-test). Scale bar: 20 μm.

FIGS. 41A-41K show Budesonide (Bud) inhibits Hh pathway activity induced by various stimuli and does not compete with Cyc for binding Smo. (A) The chemical structure of Bud. (B-C) representative images (B) and quantification of Smo ciliary localization (C) in Smo::EGFP/Ivs::tagRFPT cells treated with Shh and varying concentrations of Bud. (D) Measurement of Hh pathway activity in cells treated with Bud only or Bud followed by Shh. (E) Dose dependent inhibition of Hh pathway activity by Bud on Shh or SAG treatment, 50 nM or 1 μM, respectively. (F-G) representative images (F) and quantification of Smo::EGFP or SmoM2::EGFP ciliary intensity (G) from cells treated with Bud. Bud was used at 22.2 μM in (F). (H) Bud's dose dependent inhibition of Hh pathway activity induced by overexpression of wildtype Smo and SmoM2 respectively. (I) Measurement of Hh pathway activity in suFU−/− cells treated with Bud and FA respectively. DMSO and SAG were used as negative control and GANT61 was positive control. (J-K) representative images (J) and quantification of Bodipy-Cyc intensity in Smo expressing cos ? cells (K) treated with Bodipy-Cyc and Bud. Vehicle was used for comparison. Bud was used at 200 μM in (J). All quantitative data represent mean (±S.D.) from either quadruplicated samples (imaging assays) or triplicate experiments (Gli-luciferase assays). Quantifications of ciliary localization involved over a thousand cells per sample whereas 50-100 Smo expressing cells were analyzed in each treatment for Bodipy-Cyc competition assay. Scale bar: 5 μm.

FIGS. 42A-42C show GDC0449 and Bud combinatorially inhibit Smo-directed Hedgehog signaling. (A-B) Quantification of Smo ciliary localization (A) and representative images (B) of Smo::EGFP/Ivs::tagRFPT cells treated with GDC0449 and Shh in the presence or absence of 10 μM Bud. In (A), GDC0449 was co-applied at 1.6 nM with Shh and Shh+Bud respectively. (C) GDC0449 dose-dependent inhibition of Shh stimulated Hh pathway activity in the presence or absence of 10 μM Bud. Data plotted are mean (±S.D.) from four biological replicates (B) analyzing over a thousand of cells or three biological replicates(C). Scale bar: 5 μm.

FIGS. 43A-43N show a high content screen for compounds driving Smo ciliary tranlocation. (A) A 500 nM solution of the small molecule Smo agonist SAG was serially diluted in low serum medium Smo::EGFP/Ivs::tagRFPT cells were treated with varying concentrations of SAG. Here and elsewhere, the Ivs::tagRFPT images were shifted leftwards by 5 pixels to show both Smo and Ivs signals in merged images. Scale bar: 5 μm. (B) Multi-parametric quantification of data sets in the high content assay: mean (±S.D.) from duplicate experiments are presented. (C) A 15 μM Cyc solution was serially diluted in low serum medium and introduced to Smo::EGFP/Ivs::tagRFPT cells. Scale bar: 10 μm. (D) Multi-parametric quantifications of data: mean from duplicate experiments. (E) A 200 μM solution of FKL was serially diluted in low serum medium and applied to Smo::EGFP/Ivs::tagRFPT cells. Note the lengthened cilium of treatment with FKL evident by lengthened Ivs::tagRFPT domain. Scale bar: 10 μm. (F) Multi-parametric quantification of data sets: mean (±S.D) from four replicates. (G) A Plot of relative Smo::EGFP+ cilium count normalized to cell number for putative agonist screen. Each dot represents the measurement of over a thousand cells in each well. Compound libraries were assayed at 10 μM. SANT-1 and SAG were used at 1 μM and Cyc at 10 μM. (H) The chemical structure of LY294002. (I) Representative images of Smo::EGFP/Ivs::tagRFPT cells treated with 30 μM LY294002 in comparison with DMSO vehicle only. Scale bar: 10 μm. (J) Quantification of Smo::EGFP positive cilia upon treatment with varying concentrations of LY294002 (red) and dose dependent inhibition of Shh ligand driven pathway activity by LY294002 (blue). (K) High content quantification of various parameters of treatment with varying concentrations of LY294002. Means (±S.D) from four replicates were presented. (L) Structures of all glucocorticoids (in alphabetical order) demonstrated to promote Smo ciliary accumulation. (M) High content image analyses of Smo::EGFP/Ivs::tagRFPT cells treated with a different concentrations of FA. Measurements were performed in quadruplicate scoring several hundred cells in each sample. Data display mean values (±S.D). (N) Wnt signaling activity as measured by Top-flash signal was not affected when FA was applied alone, simultaneously with Wnt3a ligand or whenreporter cells were pre-treated with FA prior to Wnt3a ligand addition. Measurements were performed in quadruplicate. Data display the mean (±S.D.).

FIGS. 44A-44E show analyses of Smo ciliary accumulation induced by overexpression or FA. (A) Comparison of the dose-response required for activating the Hh pathway when over-expressing Smo (red curve) or GFP (green curve) in reporter cells. Experiments were performed in quadruplicate. Data display the mean (±S.D.). (Inset) A representative image of a Smo over-expressing cell line shows Smo accumulation in the PC in the absence of ligand. Scale bar: 10 μm. (B) Representative images taken immediately, 8 hours, and 24 hours after FA(10 μM) withdrawal. (C) Time lapse plots of the fluorescent intensity of Smo::EGFP and Ivs::tagRFPT at the PC following FA(10 μM) withdrawal. Data display the mean (±S.D.) calculated from four repeats measuring several hundred cells in each sample. Scale bar: 5 μm. (D) Graphs of dose dependent reduction of FA driven Smo accumulation within the PC on increasing levels of SANT-1 and GDC0449. Mean (±S.D.) of relative number of Smo+PC calculated from analysis of several hundred cells in quadruplicate treatments. (E) Representative images. Scale bar: 5 μm.

FIGS. 45A-45D show FA confers resistance to Hh pathway inhibition by the Smo antagonists Cyc and SANT-1. (A-B) Cyc (A) or SANT-1 (B) mediated dose dependent inhibition of Shh ligand induced Hh pathway activation in the presence or absence of 10 μM FA. (C) SANT-1 mediated dose-dependent inhibition of Smo accumulation at the PC in response to Shh ligand stimulation in the presence or absence of 10 μM FA. (D) Representative images quantified in (C). SANT-1 was applied at 370 nM. Measurements represent several hundred cells in quadruplicate samples. Data plotted display the mean (±S.D.). Scale bar: 5 μm.

FIG. 46A-46R show analyses of GC Smo antagonists, Ciclesonide (Cic) and Bud. (A) Cic structure. (B) representative images of Shh treated Smo::EGFP/Ivs::tagRFPT cells with DMSO or Cic at 30 μM. Scale bar: 5 μm. (C) Quantification of Smo ciliary localization of cells treated with Shh and various doses of Cic. Measurements represent several hundred cells in quadruplicate samples. Data plotted display the mean (±S.D.). (D) Cic dose-dependent inhibition of Hh pathway activity measured by a Gli responsive luciferase reporter activity in NIH/3T3 cells. Data plotted display the mean (±S.D.) of triplicate samples. (E) Representative images of Smo::EGFP/Ivs::tagRFPT cells treated with 100 μM Bud and 5 μM Cyc or varying doses of SAG. DMSO was a vehicle control for comparison. Scale bar: 5 μm. (F) Quantification of Bud's dose-dependent inhibition of Smo::EGFP ciliary intensity induced by SAG or Cyc. Measurements represent several hundred cells in quadruplicate samples. Data plotted display the mean (±S.D.). (G) Wnt signaling activity in a Top-flash reporter assay was not affected when Bud was applied alone, simultaneously with Wnt3a ligand or by pre-treating reporter cells prior to Wnt3a ligand addition. Measurements were performed in quadruplicate. Data display the mean (±S.D.). (H and J) Representative images of Smo::EGFP and SmoM2::EGFP overexpressing cells treated with 370 nM SANT-1 (H) or 370 nM GDC0449 (J). DMSO was used as a control for comparison. Scale bar: 5 μm. (I and K) Quantification of ciliary intensity of Smo::EGFP and SmoM2::EGFP for cells treated with varying concentrations of SANT-1(I) or GDC0449(K). Data plotted display the mean (±S.D.) from triplicate experiments. (L) Representative images of Smo::EGFP and SmoM2::EGFP overexpressing cells treated with 100 μM Cic. DMSO was used as a control for comparison. Both Ivs::tagRFPT and acetylated tubulin (acet-tub) were examined as markers enriched in the PC. Scale bar: 5 μm. (M-O) Quantification of Smo::EGFP and SmoM2::EGFP ciliary intensity (M), Ivs::tagRFPT ciliary intensity (N), and Ivs::tagRFPT positive cilium count (O). Measurements represent several hundred cells in quadruplicate samples. Data plotted display the mean (±S.D.). (P) High content quantification of Smo::EGFP/Ivs::tagRFPT cells treated with Shh and varying concentrations of Bud. Measurements represent several hundred cells in quadruplicate samples. Data plotted display the mean (±S.D.). (Q) Comparative analysis of Arl13b::tagRFPT and acet-tub in the PC of cells treated with 100 μM Bud or DMSO vehicle. Scale bar: 5 μm. (R) Quantitative analyses of Arl13b::tagRFPT positive primary cilia upon Bud treatment. Measurements represent several hundred cells in quadruplicate samples. Data plotted display the mean (±S.D.).

### DETAILED DESCRIPTION

In one aspect, described herein is a method of modulating a Hedgehog signaling pathway in a cell, the method comprising contacting the cell with a compound described herein.

The cell can be contacted with the compound in a cell culture e.g., in vitro or ex vivo, or the compound can be administrated to a subject, e.g., in vivo. The term “contacting” or “contact” as used herein in connection with contacting a cell includes subjecting the cell to an appropriate culture media which comprises the indicated compound. Where the cell is in vivo, “contacting” or “contact” includes administering the compound in a pharmaceutical composition to a subject via an appropriate administration route such that the compound contacts the cell in vivo. For in vivo methods, a therapeutically effective amount of a compound can be administered to a subject. Methods of administering compounds to a subject are known in the art and easily available to one of skill in the art.

As used herein, the term “modulate,” with reference to the Hedgehog signaling pathway, means to regulate positively or negatively the normal functioning of a component in the Hedgehog signaling pathway. Thus, the term modulate can be used to refer to an increase, decrease, masking, altering, overriding or restoring the normal functioning of a component in the Hedgehog signaling pathway.

The term “Hedgehog signaling pathway”, “Hedgehog pathway” and “Hedgehog signal transduction pathway” are all used to refer to the chain of events normally mediated by Hedgehog, smoothened, Ptch1, and Gli, among others, and resulting in a changes in gene expression and other phenotypic changes typical of Hedgehog activity. Activating a downstream component can activate the Hedgehog pathway even in the absence of a Hedgehog protein. For example, overexpression of smoothened will activate the pathway in the absence of Hedgehog, Gli and Ptch1 gene expression are indicators of an active Hedgehog-signaling pathway. Accordingly, compounds described herein can be used to overcome an inappropriate increase in Hedgehog signal transduction, whether said increase in signal transduction is the result in a mutation/lesion in a component of the Hedgehog signaling pathway (e.g., Ptch1, Gli1, Gli3, smoothened, etc) or whether said increase in signal transduction occurs in the context of a cell which does not comprise a mutation/lesion in a component of the Hedgehog signaling pathway (e.g., a wildtype cell with respect to components of the Hedgehog signaling pathway). Thus, in some embodiments, the cell has a phenotype of smoothened gain-of-function, Hedgehog gain-of-function, patched (Ptc) loss-of-function, Gli gain-of-function, and/or over expression of Hedgehog ligands.

The term “smoothened gain-of-function” refers to an aberrant modification or mutation of a smo gene, or an increased level of expression of the gene, which results in a phenotype that resembles contacting a cell with a Hedgehog protein, e.g., aberrant activation of a Hedgehog pathway. While not wishing to be bound by any particular theory, it is noted that Ptch1 may not signal directly into the cell, but rather modulates the activity of smoothened, another membrane bound protein located downstream of Ptch1 in Hedgehog signaling (Mango et al., (1996) Nature 384: 177-179; Taipale et al. (2002) Nature 418, 892-896). The gene smo is a segment polarity gene required for the correct patterning of every segment in Drosophila (Alcedo et al., (1996) Cell 86:221232). Human homologs of smo have been identified. See, for example, Stone et al. (1996) Nature 384:129-134, and GenBank accession U84401. The smoothened gene encodes an integral membrane protein with characteristics of heterotrimeric G-protein-coupled receptors; i.e., 7-transmembrane regions. This protein shows homology to the Drosophila Frizzled (Fz) protein, a member of the wingless pathway. Ptc is a Hh receptor. Cells that express Smo fail to bind Hh, indicating that smo does not interact directly with Hh (Nusse, (1996) Nature 384: 119120). Rather, the binding of Sonic Hedgehog (SHH) to its receptor, PTCH. is thought to prevent normal inhibition by PTCH of smoothened. Activating smoothened mutations are known to occur in sporadic basal cell carcinoma (Xie, et al., Nature, 1998, 391: 90-92), and in primitive neuroectodermal tumors of the central nervous system (Reifenberger, et al., Cancer Res., 1998, 58:1798-1803).

The term “Hedgehog gain-of-function” refers to an aberrant modification or mutation of a Ptch1 gene, Hedgehog gene, or smoothened gene, or a decrease (or loss) in the level of expression of such a gene, which results in a phenotype which resembles contacting a cell with a Hedgehog protein, e.g., aberrant activation of a Hedgehog pathway. The gain-of-function may include a loss of the ability of the Ptch1 gene product to regulate the level of expression of Ci homolog genes, e.g., Gli 1, Gli2, and Gli3. The term “Hedgehog gain-of-function” is also used herein to refer to any similar cellular phenotype (e.g., exhibiting excess proliferation) that occurs due to an alteration anywhere in the Hedgehog signal transduction pathway, including, but not limited to, a modification or mutation of Hedgehog itself. For example, a tumor cell with an abnormally high proliferation rate due to activation of the Hedgehog signalling pathway would have a “Hedgehog gain-of-function” phenotype, even if Hedgehog is not mutated in that cell.

The term “patched loss-of-function” refers to an aberrant modification or mutation of a Ptch1 gene, or a decreased level of expression of the gene, which results in a phenotype which resembles contacting a cell with a Hedgehog protein, e.g., aberrant activation of a Hedgehog pathway. The loss-of-function may include a loss of the ability of the Ptch1 gene product to regulate the level of expression or activity of Ci homolog genes, e.g., Gli1, Gli2, and Gli3. The term ‘Ptch1 loss-of function’ is also used herein to refer to any similar cellular phenotype (e.g., exhibiting excess proliferation) that occurs due to an alteration anywhere in the Hedgehog signal transduction pathway, including, but not limited to, a modification or mutation of Ptch1 itself. For example, a tumor cell with an abnormally high proliferation rate due to activation of the Hedgehog signalling pathway would have a “Ptch1 loss-of-function” phenotype, even if Ptch1 is not mutated in that cell.

The term “Gli gain-of-function” refers to an aberrant modification or mutation of a Gli gene, or an increased level of expression of the gene, which results in a phenotype that resembles a cell responding to a Hedgehog protein, e.g., aberrant activation of a Hedgehog pathway.

The vertebrate family of Hedgehog genes includes three members that exist in mammals, known as Desert (Dhh), Sonic (Shh) and Indian (Ihh) Hedgehogs, all of which encode secreted proteins. These various Hedgehog proteins consist of a signal peptide, a highly conserved N-terminal region, and a more divergent C-terminal domain. Biochemical studies have shown that autoproteolytic cleavage of the Hh precursor protein proceeds through an internal thioester intermediate which subsequently is cleaved in a nucleophilic substitution. It is likely that the nucleophile is a small lipophilic molecule which becomes covalently bound to the C-terminal end of the N-peptide, tethering it to the cell surface. The biological implications are profound. As a result of the tethering, a high local concentration of N-terminal Hedgehog peptide is generated on the surface of the Hedgehog producing cells. It is this N-terminal peptide which is both necessary and sufficient for short- and long-range Hedgehog signaling activities.

An inactive Hedgehog signaling pathway is where the transmembrane protein receptor Patched (Ptc) inhibits the activity of Smoothened (Smo), a seven transmembrane protein. The transcription factor Gli, a downstream component of Hh signaling, is processed to a repressor form and nuclear accumulation of activator forms prevented prevented through interactions with cytoplasmic proteins, including Fused and Suppressor of fused (Sufu). As a consequence, transcriptional activation of Hedgehog target genes is repressed. Activation of the pathway is initiated through binding of any of the three mammalian ligands (Dhh, Shh or Ihh) to Ptc. Ligand binding results in a reversal of the repression of Smo, thereby activating a cascade that leads to the translocation of the active form of the transcription factor Gli to the nucleus. Nuclear Gli activates target gene expression, including Ptc and Gli itself. Increased levels of Hedgehog signaling are sufficient to initiate cancer formation and are required for tumor survival.

In some embodiments of this and other aspects described herein, the cell is a cancer cell. As used herein, the term “cancer cell” refers to a cell undergoing early, intermediate or advanced stages of multi-step neoplastic progression as described, for example, in H. C. Pilot (1978) in “Fundamentals of Oncology,” Marcel Dekker (Ed.), New York pp 15-28. The features of early, intermediate and advanced stages of neoplastic progression have been described using microscopy. Cancer cells at each of the three stages of neoplastic progression generally have abnormal karyotypes, including translocations, inversion, deletions, isochromosomes, monosomies, and extra chromosomes. A cell in the early stages of malignant progression is referred to as “hyperplastic cell” and is characterized by dividing without control and/or at a greater rate than a normal cell of the same cell type in the same tissue. Proliferation may be slow or rapid but continues unabated. A cell in the intermediate stages of neoplastic progression is referred to as a “dysplastic cell.” A dysplastic cell resembles an immature epithelial cell, is generally spatially disorganized within the tissue and loses its specialized structures and functions. During the intermediate stages of neoplastic progression, an increasing percentage of the epithelium becomes composed of dysplastic cells. “Hyperplastic” and “dysplastic” cells are referred to as “pre-neoplastic” cells. In the advanced stages of neoplastic progression a dysplastic cell become a “neoplastic” cell. Neoplastic cells are typically invasive i.e., they either invade adjacent tissues, or are shed from the primary site and circulate through the blood and lymph to other locations in the body where they initiate secondary cancers. As used herein, the term “cancer cell” encompass both pre-malignant and malignant cancer cells.

In some embodiments of this and other aspects described herein, the compound is formula (I):

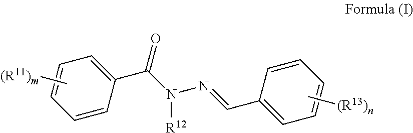

wherein:
R¹¹ is independently for each occurrence optionally substituted alkyl, optionally substituted alkenyl, optionally substituted alkynyl, optionally substituted cyclyl, optionally substituted heterocyclyl, optionally substituted aryl, optionally substituted heteroaryl, OR¹⁴, NO₂, CN, CF₃, halo, C(O)R¹⁴, CO₂R¹⁴, SOR¹⁴, SO₂R¹⁴, or N(R¹⁵)₂;
R¹² is H, optionally substituted alkyl, optionally substituted alkenyl, optionally substituted alkynyl, optionally substituted cyclyl, optionally substituted heterocyclyl, optionally substituted aryl, optionally substituted heteroaryl, OR¹⁴, C(O)R¹⁴, or CO₂R¹⁴;
R¹³ is independently for each occurrence optionally substituted alkyl, optionally substituted alkenyl, optionally substituted alkynyl, optionally substituted cyclyl, optionally substituted heterocyclyl, optionally substituted aryl, optionally substituted heteroaryl, OR¹⁴, NO₂, CN, CF₃, halo, C(O)R¹⁴, CO₂R¹⁴, SOR¹⁴, SO₂R¹⁴, or N(R¹⁵)₂;
R¹⁴ is independently for each occurrence optionally substituted alkyl, optionally substituted alkenyl, optionally substituted alkynyl, optionally substituted cyclyl, optionally substituted heterocyclyl, optionally substituted aryl, or optionally substituted heteroaryl;
R¹⁵ is independently for each occurrence H, optionally substituted alkyl, optionally substituted alkenyl, optionally substituted alkynyl, optionally substituted cyclyl, optionally substituted heterocyclyl, optionally substituted aryl, optionally substituted heteroaryl, C(O)R¹⁴, or CO₂R¹⁴;
m is 0, 1, 2, 3, 4, or 5;
n is 0, 1, 2, 3, 4, or 5; and
isomers and pharmaceutically acceptable salts thereof.

In some embodiments, m is 1 or 2.

When present, each R¹¹ can be independently a halogen or OR¹⁴. Exemplary R¹⁴ include, but are not limited to H, optionally substituted C₁-C₆ alkyl. Accordingly, in some embodiments, R¹¹ is Cl, OH or OCH₃.

In some embodiments, n is 1.

When present, each R¹³ can be independently a halogen, optionally substituted C₁-C₆alkyl, or NO₂, N(R¹⁵)₂. Exemplary R¹⁵ substituents include H and C₁-C₆ alkyl. Accordingly, in some embodiments, R¹³ is F, NO₂, N(CH₂CH₃)₂, N(CH₃)₂, or CH(CH₂CH₃)₂.

In some embodiments, R¹² is H or optionally substituted C₁-C₆alkyl.

In some embodiments, a compound of formula (I) is of formula (Ia): or (Ib):

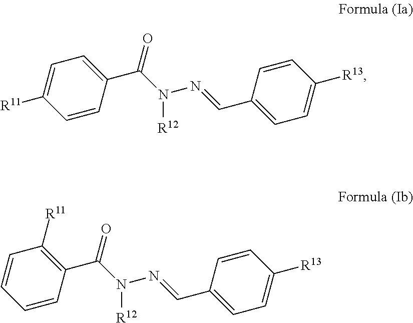

wherein R¹¹, R¹² and R¹³ are as defined above.

Some exemplary compounds of formula (I) include

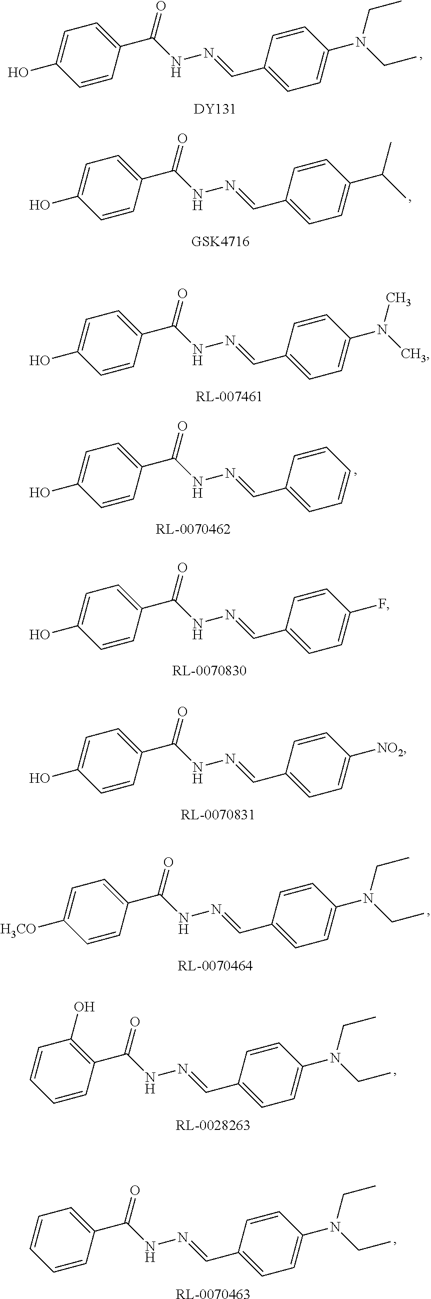

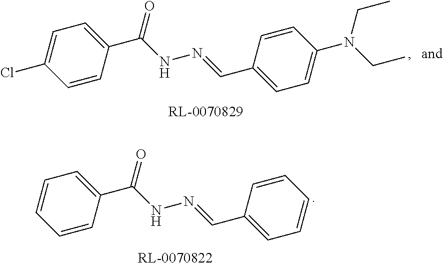 [SMILES: CCN(CC)c1ccc(/C=N/NC(=O)c2ccc(Cl)cc2)cc1 ; "RL-0070829" ; O=C(N/N=C/c1ccccc1)c1ccccc1 ; "RL-0070822"]

In some embodiments of this and other aspects described herein, the compound is formula (I):

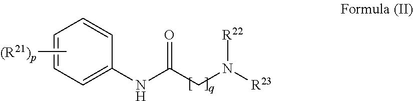

wherein:
R²¹ is independently for each occurrence optionally substituted alkyl, optionally substituted alkenyl, optionally substituted alkynyl, optionally substituted cyclyl, optionally substituted heterocyclyl, optionally substituted aryl, optionally substituted heteroaryl, OR²⁴, NO₂, CN, CF₃, halo, OC(O)R²⁴, C(O)R²⁴, CO₂R²⁴, C(O)N(R₂₄)₂, SOR²⁴, SO₂R²⁴, N(R²⁴)₂, NHC(O)R²⁴, NHCO₂R²⁴, or two R²¹s together with the carbons they are attached to form an optionally substituted 5-8 member cyclyl or an optionally substituted 5-8 member heterocyclyl;
R²² and R²³ are independently H, optionally substituted alkyl, optionally substituted alkenyl, optionally substituted alkynyl, optionally substituted cyclyl, optionally substituted heterocyclyl, optionally substituted aryl, optionally substituted heteroaryl, optionally substituted —(CH₂)ₜ—R²⁴, or R²² and R²³ together with the nitrogen they are attached to form an optionally substituted 5-8 membered ring;
R²⁴ is independently for each occurrence optionally substituted alkyl, optionally substituted alkenyl, optionally substituted alkynyl, optionally substituted cyclyl, optionally substituted heterocyclyl, optionally substituted aryl, or optionally substituted heteroaryl;
p is 0, 1, 2, 3, 4, or 5;
q is 1, 2, 3, 4, 5, or 6;
t is 1, 2, 3, 4, 5, or 6; and
isomers and pharmaceutically acceptable salts thereof.

In some compounds of formula (II), p can be 1 or 2.

In some compounds of formula (II), q can be 1 or 2.

In some embodiments, t can be 1, 2, or 3.

When R²² and R²³ do not form a ring, they both can be the same or both different. For example, at least one of or both of R²² and R²³ can be an optionally substituted C₁-C₆alkyl, at least one of R²² and R²³ can be —(CH₂)—R²⁴, or any combinations thereof. In some compounds of formula (II), at least one of at least one of R²⁴ and R²³ is methyl. I

In some embodiments, one of R²² and R²³ is methyl and the other is a butyl.

In some embodiments, one of R²² and R²³ can be an optionally substituted C₁-C₆alkyl and the other —(CH₂)ₜ—R²⁴. In one embodiment of this, one of R²² and R²³ is methyl and the other is —(CH₂)ᵣR²⁴. In some embodiments, at least one of R²² and R²³ is —(CH₂)ᵣR²⁴ and R²⁴ is optionally substituted aryl or heteroaryl. In one embodiment, at least one of R²² and R²³ is —CH₂—R²⁴ and R²⁴ is phenyl.

In some embodiments, a compound of formula (II) is of formula (IIa):

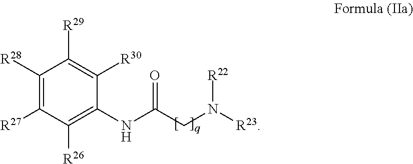

Variables R²², R²³ and q are as defined above for formula (II).

R²⁶ can be H, optionally substituted alkyl, optionally substituted alkenyl, optionally substituted alkynyl, optionally substituted cyclyl, optionally substituted heterocyclyl, optionally substituted aryl, optionally substituted heteroaryl, OR²⁴, NO₂, CN, CF₃, halo, OC(O)R²⁴, C(O)R²⁴, CO₂R²⁴, C(O)N(R²⁴)₂, SOR²⁴, SO₂R²⁴, N(R²⁴)₂, NHC(O)R²⁴, or NHCO₂R²⁴. In some embodiments, R²⁶ can be halo or optionally substituted C₁-C₆alkyl. In some embodiments, R²⁶ is H, F, Cl, or methyl.

R²⁷ can be H, optionally substituted alkyl, optionally substituted alkenyl, optionally substituted alkynyl, optionally substituted cyclyl, optionally substituted heterocyclyl, optionally substituted aryl, optionally substituted heteroaryl, OR²⁴, NO₂, CN, CF₃, halo, OC(O)R²⁴, C(O)R²⁴, CO₂R²⁴, C(O)N(R²⁴)₂, SOR²⁴, SO₂R²⁴, N(R²⁴)₂, NHC(O)R²⁴, or NHCO₂R²⁴. In some embodiments, R²⁷ can be halo, optionally substituted C₁-C₆alkyl, or OR²⁴, wherein R²⁴ is optionally substituted C₁-C₆ alkyl. In some embodiments, R²⁴ is H, Cl, methyl, or OCH₃.

R²⁸ can be H, optionally substituted alkyl, optionally substituted alkenyl, optionally substituted alkynyl, optionally substituted cyclyl, optionally substituted heterocyclyl, optionally substituted aryl, optionally substituted heteroaryl, OR²⁴, NO₂, CN, CF₃, halo, OC(O)R²⁴, C(O)R²⁴, CO₂R²⁴, C(O)N(R²⁴)₂, SOR²⁴, SO₂R²⁴, N(R²⁴)₂, NHC(O)R²⁴, or NHCO₂R²⁴. In some embodiments, R²⁸ can be H, halo, optionally substituted C₁-C₆alkyl, or CO₂R²⁴, where R²⁴ is optionally substituted C₁-C₆alkyl. In some embodiments, R²⁸ is H, Br, F, methyl, CO₂CH₂CH₃.

In some embodiments, R²⁷ and R²⁸ together with the carbon atoms they are attached can form a 5-8 membered cyclyl or heterocyclyl, which can be optionally substituted. In some embodiments, R²⁷ and R²⁸ together with the carbon atoms they are attached can form a 5-membered heterocylcyl which can be optionally substituted. In one embodiments R²⁷ and R²⁸ together with the

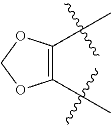 [SMILES: *C1=C(*)OCO1]

carbons they are attached to form

R²⁹ can be H, optionally substituted alkyl, optionally substituted alkenyl, optionally substituted alkynyl, optionally substituted cyclyl, optionally substituted heterocyclyl, optionally substituted aryl, optionally substituted heteroaryl, OR²⁴, NO₂, CN, CF₃, halo, OC(O)R²⁴, C(O)R²⁴, CO₂R²⁴, C(O)N(R²⁴)₂, SOR²⁴, SO₂R²⁴, N(R²⁴)₂, NHC(O)R²⁴, or NHCO₂R²⁴. In some embodiments, R²⁹ can be H, halo, optionally substituted C₁-C₆alkyl, or OR²⁴, wherein R²⁴ is optionally substituted C₁-C₆ alkyl. In some embodiments, R²⁹ is H, Cl, methyl or OCH₃.

In some embodiments, R²⁸ and R²⁹ together with the carbon atoms they are attached can form a 5-8 membered cyclyl or heterocyclyl, which can be optionally substituted. In some embodiments, R²⁸ and R²⁹ together with the carbon atoms they are attached can form a 5-membered ring which can be optionally substituted.

R³⁰ can be H, optionally substituted alkyl, optionally substituted alkenyl, optionally substituted alkynyl, optionally substituted cyclyl, optionally substituted heterocyclyl, optionally substituted aryl, optionally substituted heteroaryl, OR²⁴, NO₂, CN, CF₃, halo, OC(O)R²⁴, C(O)R²⁴, CO₂R²⁴, C(O)N(R²⁴)₂, SOR²⁴, SO₂R²⁴, N(R²⁴)₂, NHC(O)R²⁴, or NHCO₂R²⁴. In some embodiments, R³⁰ can be H, optionally substituted C₁-C₆alkyl or OR²⁴, wherein R²⁴ is an optionally substituted C₁-C₆alkyl. In some embodiments, R³⁰ is H, methyl or OCH₃.

It is to be understood that R²⁶, R²⁷, R²⁸, R²⁹ and R³⁰ can all be the same, all different or any combinations of same and different.

In some embodiments, a compound of formula (II) is of formula (IIb):

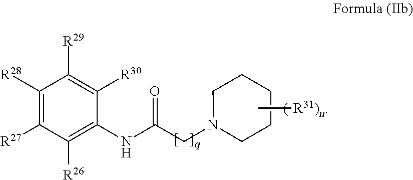

Variables R²⁶-R³⁰ and q are as defined above for formula (IIa).

R³¹ is independently for each occurrence optionally substituted alkyl, optionally substituted alkenyl, optionally substituted alkynyl, optionally substituted cyclyl, optionally substituted heterocyclyl, optionally substituted aryl, optionally substituted heteroaryl, OR²⁴, NO₂, CN, CF₃, halo, OC(O)R²⁴, C(O)R²⁴, CO₂R²⁴, C(O)N(R²⁴)₂, SOR²⁴, SO₂R²⁴, N(R²⁴)₂, NHC(O)R²⁴, or NHCO₂R²⁴. In some embodiments, R³¹ can be optionally substituted C₁-C₆alkyl or CO₂R²⁴, wherein R²⁴ is optionally substituted C₁-C₆alkyl. In some embodiments, R³¹ is methyl or CO₂CH₂CH₃.

Variable u can be 0, 1, 2, 3, 4, or 5. In some embodiments, u is 0, 1, 2, or 3.

In some embodiments, a compound of formula (II) is of formula (IIc):

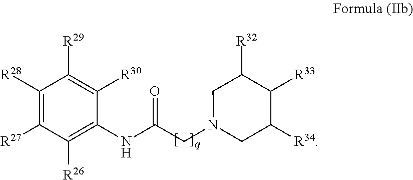

R²⁶-R³⁰ and variable q are as defined above for formula (IIa).

R³² can be H, optionally substituted alkyl, optionally substituted alkenyl, optionally substituted alkynyl, optionally substituted cyclyl, optionally substituted heterocyclyl, optionally substituted aryl, optionally substituted heteroaryl, OR²⁴, NO₂, CN, CF₃, halo, OC(O)R²⁴, C(O)R²⁴, CO₂R²⁴, C(O)N(R²⁴)₂, SOR²⁴, SO₂R²⁴, N(R²⁴)₂, NHC(O)R²⁴, or NHCO₂R²⁴. In some embodiments, R³² can be optionally substituted C₁-C₆alkyl. In one some embodiments, R³² is H or methyl.

R³³ can be H, optionally substituted alkyl, optionally substituted alkenyl, optionally substituted alkynyl, optionally substituted cyclyl, optionally substituted heterocyclyl, optionally substituted aryl, optionally substituted heteroaryl, OR⁴, NO₂, CN, CF₃, halo, OC(O)R⁴, C(O)R⁴, CO₂R⁴, C(O)N(R⁴)₂, SOR⁴, SO₂R⁴, N(R⁴)₂, NHC(O)R⁴, or NHCO₂R⁴. In some embodiments, R³³ can be optionally substituted C₁-C₆alkyl. In one some embodiments, R³³ is H or methyl.

R³⁴ can be H, optionally substituted alkyl, optionally substituted alkenyl, optionally substituted alkynyl, optionally substituted cyclyl, optionally substituted heterocyclyl, optionally substituted aryl, optionally substituted heteroaryl, OR⁴, NO₂, CN, CF₃, halo, OC(O)R⁴, C(O)R⁴, CO₂R⁴, C(O)N(R⁴)₂, SOR⁴, SO₂R⁴, N(R⁴)₂, NHC(O)R⁴, or NHCO₂R⁴. In some embodiments, R³⁴ can be optionally substituted C₁-C₆alky or CO₂R⁴, wherein R⁴ is optionally substituted C₁-C₆alkyl. In some embodiments, R¹³ is H, methyl, or CO₂CH₂CH₃.

In some embodiments, a compound of formula (II) is of formula (IIc):

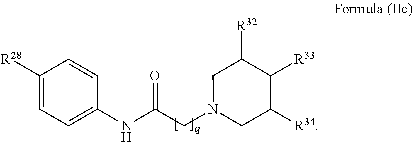

In some embodiments, a compound of formula (II) is of formula (IId):

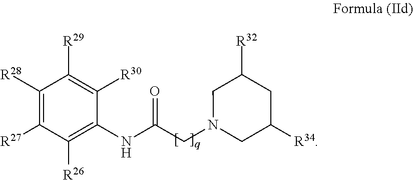

In some embodiments, a compound of formula (II) is of formula (IIe):

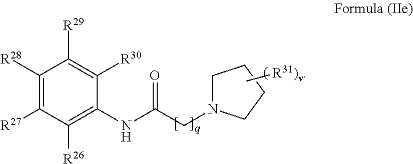

Variable R²⁶-R³¹ and q are as defined above.

Variable v can be 0, 1, 2, 3, or 4. In some embodiments, v is 0.

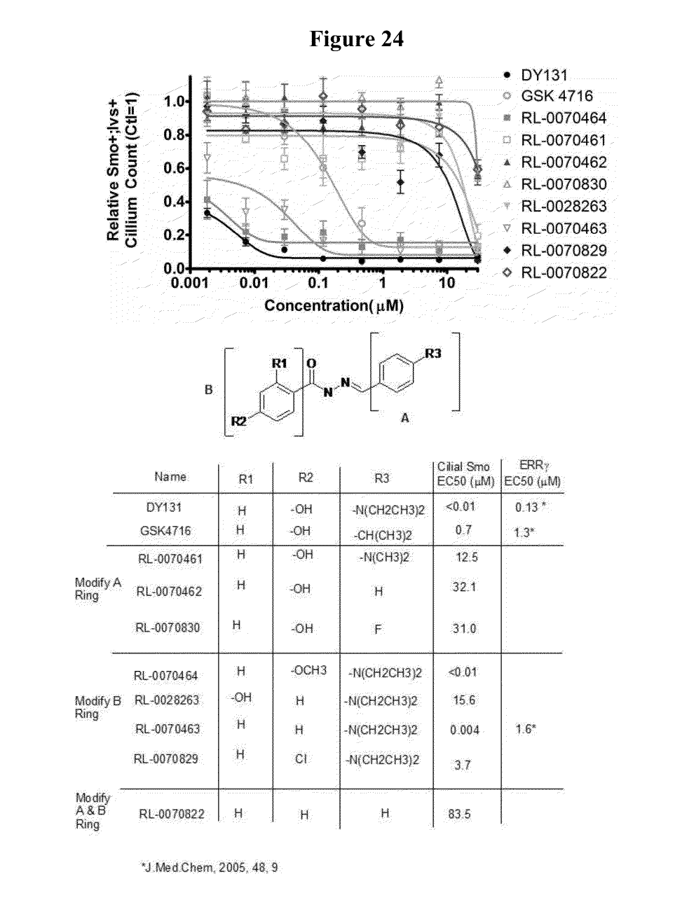

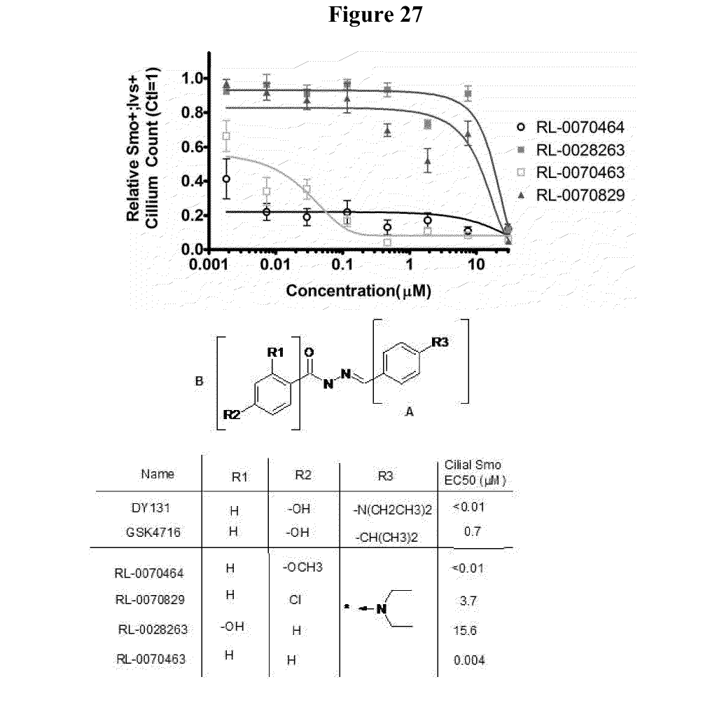

Some exemplary compounds of formula (II) are shown in FIGS. 24-36.

In some embodiments, a compound is an analog, derivative or prodrug of a compound of formula (I) or formula (II)

Without limitations, a compound of described herein can exist as a pure stereoisomer, or as a racemic mixture.

Hedgehog pathway antagonists that induce Smo accumulation to the PC can also trigger a hypersensitivity to pathway stimulation when inhibitory levels fall. Accordingly, in some embodiments of this and other aspects described herein, a compound described herein inhibits the translocation of smoothened (Smo) to the primary cilium and/or the accumulation of Smo in the primary cilium. Compounds that inhibit the translocation of smoothened (Smo) to the primary cilium and/or the accumulation of Smo in the primary cilium are also referred to as Smo antagonists herein.

In some embodiments, a compound that inhibits the accumulation and/or translocation of smoothened to the primary cilium is a compound of formula (I) or formula (II).

In some embodiments of this and other aspects described herein, the compound increases the translocation of smoothened (Smo) to the primary cilium and/or the accumulation of Smo in the primary cilium. Compounds that increase the accumulation of Smo in the primary cilium can be used to sensitize and/or prime the Hedgehog pathway to activation. Without wishing to be bound by a theory, a compound that increases Smo accumulation in the primary cilium may not activate the Hedgehog pathway but can sensitize cells to Hedgehog pathway activation. Additionally these compounds can reduce the inhibitory properties of antagonists of Smo signaling. To sensitize and/or prime the Hedgehog pathway to activation means to make the Hedgehog pathway more sensitive to activation.

In some embodiments, a compound that increases the translocation of smoothened (Smo) to the primary cilium and/or the accumulation of Smo in the primary cilium is a glucocorticoid. As used herein, the term “glucocorticoid” means a steroid hormone glucocorticoid. “Glucocorticoids” are agonists for the glucocorticoid receptor. Compounds that mimic glucocorticoids are also considered to be glucocorticoids herein. Exemplary glucocorticoid receptor agonists include, but are not limited to, aldosternone, beclomethasone, beclomethasone dipropionate, betametahasone, betametahasone-21-phosphate disodium, betametahasone valerate, budesonide (also referred to as Bud herein), clobetasol, clobetasol propionate, clobetasone butyrate, clocortolone pivalate, cortisol, cortisteron, cortisone, dexamethasone, dexamethasone acetate, dexamethasone sodium phosphate, diflorasone diacetate, flucinonide, fludrocortisones acetate, flumethasone, flunisolide, flucionolone acetonide, fluticasone furate, fluticasone propionate, halcinonide, halpmetasone, hydrocortisone, hydroconrtisone acetate, hydrocortisone succinate, 16α-hydroxyprednisolone, isoflupredone acetate, medrysone, methylprednisolone, prednacinolone, predricarbate, prednisolone, prednisolone acetate, prednisolone sodium succinate, prednisone, triamcinolone, triamcinolone, and triamcinolone diacetate.

As used herein, glucocorticoid is intended to include, for example, the following generic and brand name corticosteroids: cortisone (CORTONE ACETATE, ADRESON, ALTESONA, CORTELAN, CORTISTAB, CORTISYL, CORTOGEN, CORTONE, SCHEROSON); dexamethasone-oral (DECADRON-ORAL, DEXAMETH, DEXONE, HEXADROL-ORAL, DEXAMETHASONE INTENSOL, DEXONE 0.5, DEXONE 0.75, DEXONE 1.5, DEXONE 4); hydrocortisone-oral (CORTEF, HYDROCORTONE); hydrocortisone cypionate (CORTEF ORAL SUSPENSION); methylprednisolone-oral (MEDROL-ORAL); prednisolone-oral (PRELONE, DELTA-CORTEF, PEDIAPRED, ADNISOLONE, CORTALONE, DELTACORTRIL, DELTASOLONE, DELTASTAB, DI-ADRESON F, ENCORTOLONE, HYDROCORTANCYL, MEDISOLONE, METICORTELONE, OPREDSONE, PANAAFCORTELONE, PRECORTISYL, PRENISOLONA, SCHERISOLONA, SCHERISOLONE); prednisone (DELTASONE, LIQUID PRED, METICORTEN, ORASONE 1, ORASONE 5, ORASONE 10, ORASONE 20, ORASONE 50, PREDNICEN-M, PREDNISONE INTENSOL, STERAPRED, STERAPRED DS, ADASONE, CARTANCYL, COLISONE, CORDROL, CORTAN, DACORTIN, DECORTIN, DECORTISYL, DELCORTIN, DELLACORT, DELTADOME, DELTACORTENE, DELTISONA, DIADRESON, ECONOSONE, ENCORTON, FERNISONE, NISONA, NOVOPREDNISONE, PANAFCORT, PANASOL, PARACORT, PARMENISON, PEHACORT, PREDELTIN, PREDNICORT, PREDNICOT, PREDNIDIB, PREDNIMENT, RECTODELT, ULTRACORTEN, WINPRED); triamcinoloneoral (KENACORT, ARISTOCORT, ATOLONE, SHOLOG A, TRAMACORT-D, TRI-MED, TRIAMCOT, TRISTOPLEX, TRYLONE D, U-TRI-LONE).

In some embodiment of this and other aspects described herein, the glucocorticoid is not clobetasol, clobetasol propionate, fluocinonide (flucinoide), fluticasone, fluticasone propionate, or halcinonide. [Does it contradict to 00102? Why glucocortcoids cannot be these?]

The inventors have also discovered that glucocorticoids unexpectedly induce hypersensitivity to Hh pathway stimulation. Accordingly, presented herein is a method for modulating cellular sensitivity to a Hedghog signaling in a cell, the method comprising contacting a cell with a compound described herein. In some embodiments, the Hedgehog signaling pathway can become hypersensitive to a Shh ligand relative to cell in absence of contact with a compound described herein.

The effect of a compound described herein on the Hedgehog signaling pathway can be measured by the methods described, for example, in Int. Pat. App. Pub. No. WO/2010/078290, content of which is incorporated herein by reference in its entirety.

Compounds described herein are also applicable to cell culture techniques wherein it is desirable to modulate the Hedgehog signaling pathway. Accordingly, another aspect of the invention relates to a method of modulating a differentiated state, survival, and/or proliferation of cell, the method comprising contacting the cell with a compound described herein.

### Method of Treatment

The compound described herein can be administered to a subject as part of a therapeutic application. In general, the method can be characterized as including a step of administering a therapeutically effective amount of the compound to subject in need thereof.

In one embodiment, the compounds and method can used in treating a subject afflicted with cancer, a precancerous condition and/or metastasis. As used herein, an anti-cancer treatment aims to reduce, prevent or eliminate cancer cells or the spread of cancer cells or the symptoms of cancer in the local, regional or systemic circulation. Anti-cancer treatment also means the direct treatment of tumors, for example by reducing or stabilizing their number or their size (curative effect), but also by preventing the in situ progression of tumor cells or their diffusion, or the establishment of tumors; this also includes the treatment of deleterious effects linked to the presence of such tumors, in particular the attenuation of symptoms observed in a patient or an improvement in quality of life. By “reduced” in the context of cancer is meant reduction of at least 10% in the growth rate of a tumor or the size of a tumor or cancer cell burden.

As used herein, the term “cancer” refers to an uncontrolled growth of cells that may interfere with the normal functioning of the bodily organs and systems. Cancers that migrate from their original location and seed vital organs can eventually lead to the death of the subject through the functional deterioration of the affected organs. A metastasis a cancer cell or group of cancer cells, distinct from the primary tumor location resulting from the dissemination of cancer cells from the primary tumor to other parts of the body. At the time of diagnosis of the primary tumor mass, the subject may be monitored for the presence of in transit metastases, e.g., cancer cells in the process of dissemination. As used herein, the term cancer, includes, but is not limited to the following types of cancer, breast cancer, biliary tract cancer, bladder cancer, brain cancer including Glioblastomas and medulloblastomas; cervical cancer; choriocarcinoma; colon cancer; endometrial cancer; esophageal cancer, gastric cancer; hematological neoplasms including acute lymphocytic and myelogenous leukemia; T-cell acute lymphoblastic leukemia/lymphoma; hairy cell leukemia; chronic myelogenous leukemia, multiple myeloma; AIDS-associated leukemias and adult T-cell leukemia lymphoma; intraepithelial neoplasms including Bowen's disease and Paget's disease; liver cancer; lung cancer; lymphomas including Hodgkin's disease and lymphocytic lymphomas; neuroblastomas; oral cancer including squamous cell carcinoma; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, and osteosarcoma; skin cancer including melanoma, Merkel cell carcinoma, Kaposi's sarcoma, basal cell carcinoma, and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; and renal cancer including adenocarcinoma, Wilms tumor. Examples of cancer include but are not limited to, carcinoma, including adenocarcinoma, lymphoma, blastoma, melanoma, sarcoma, and leukemia. More particular examples of such cancers include squamous cell cancer, small-cell lung cancer, non-small cell lung cancer, gastrointestinal cancer, Hodgkin's and non Hodgkin's lymphoma, pancreatic cancer, Glioblastoma, cervical cancer, ovarian cancer, liver cancer such as hepatic carcinoma and hepatoma, bladder cancer, breast cancer, colon cancer, colorectal cancer, endometrial carcinoma, salivary gland carcinoma, kidney cancer such as renal cell carcinoma and Wilms' tumors, basal cell carcinoma, melanoma, prostate cancer, vulval cancer, thyroid cancer, testicular cancer, esophageal cancer, and various types of head and neck cancer. Other cancers will be known to the artisan.

As used herein, the term “precancerous condition” has its ordinary meaning, i.e., an unregulated growth without metastasis, and includes various forms of hyperplasia and benign hypertrophy. Accordingly, a “precancerous condition” is a disease, syndrome, or finding that, if left untreated, can lead to cancer. It is a generalized state associated with a significantly increased risk of cancer. Premalignant lesion is a morphologically altered tissue in which cancer is more likely to occur than its apparently normal counterpart. Examples of pre-malignant conditions include, but are not limited to, oral leukoplakia, actinic keratosis (solar keratosis), Barrett's esophagus, atrophic gastritis, benign hyperplasia of the prostate, precancerous polyps of the colon or rectum, gastric epithelial dysplasia, adenomatous dysplasia, hereditary nonpolyposis colon cancer syndrome (HNPCC), Barrett's esophagus, bladder dysplasia, precancerous cervical conditions, and cervical dysplasia.

The compounds described herein also have wide applicability to the treatment or prophylaxis of disorders afflicting epithelial tissue, as well as in cosmetic uses, for example by altering the growth state of a treated epithelial tissue. The mode of administration and dosage regimens will vary depending on the epithelial tissue(s) that is to be treated. For example, topical formulations will be preferred where the treated tissue is epidermal tissue, such as dermal or mucosal tissues.

A method that “promotes the healing of a wound” results in the wound healing more quickly as a result of the treatment than a similar wound heals in the absence of the treatment. “Promotion of wound healing” can also mean that the method regulates the proliferation and/or growth of, inter alia, keratinocytes, or that the wound heals with less scarring, less wound contraction, less collagen deposition and more superficial surface area. In certain instances, “promotion of wound healing” can also mean that certain methods of wound healing have improved success rates, (e.g., the take rates of skin grafts,) when used together with the method of the present invention.

Despite significant progress in reconstructive surgical techniques, scarring can be an important obstacle in regaining normal function and appearance of healed skin. This is particularly true when pathologic scarring such as keloids or hypertrophic scars of the hands or lace causes functional disability or physical deformity. In the severest circumstances, such scarring may precipitate psychosocial distress and a life of economic deprivation. Wound repair includes the stages of hemostasis, inflammation, proliferation, and remodeling. The proliferative stage involves multiplication of fibroblasts and endothelial and epithelial cells. Through the use of the subject method, the rate of proliferation of epithelial cells in and proximal to the wound can be controlled in order to accelerate closure of the wound and/or minimize the formation of scar tissue.

The present treatment can also be effective as part of a therapeutic regimen for treating oral and paraporal ulcers, e.g. resulting from radiation and/or chemotherapy. Such ulcers commonly develop within days after chemotherapy or radiation therapy. These ulcers usually begin as small, painful irregularly shaped lesions usually covered by a delicate gray necrotic membrane and surrounded by inflammatory tissue. In many instances, lack of treatment results in proliferation of tissue around the periphery of the lesion on an inflammatory basis. For instance, the epithelium bordering the ulcer usually demonstrates proliferative activity, resulting in loss of continuity of surface epithelium. These lesions, because of their size and loss of epithelial integrity, dispose the body to potential secondary infection. Routine ingestion of food and water becomes a very painful event and, if the ulcers proliferate throughout the alimentary canal, diarrhea usually is evident with all its complicating factors. According to the present invention, a treatment for such ulcers which includes application of a Hedgehog antagonist can reduce the abnormal proliferation and differentiation of the affected epithelium, helping to reduce the severity of subsequent inflammatory events.

The subject method and compositions can also be used to treat wounds resulting from dermatological diseases, such as lesions resulting from autoimmune disorders such as psoriasis. Atopic dermititis refers to skin trauma resulting from allergies associated with an immune response caused by allergens such as pollens, foods, dander, insect venoms and plant toxins.

Antiproliferative preparations of compounds can be used to inhibit lens epithelial cell proliferation to prevent post-operative complications of extracapsular cataract extraction. Cataract is an intractable eye disease and various studies on a treatment of cataract have been made. But at present, the treatment of cataract is attained by surgical operations. Cataract surgery has been applied for a long time and various operative methods have been examined. Extracapsular lens extraction has become the method of choice for removing cataracts. The major medical advantages of this technique over intracapsular extraction are lower incidence of aphakic cystoid macular edema and retinal detachment. Extracapsular extraction is also required for implantation of posterior chamber type intraocular lenses that are now considered to be the lenses of choice in most cases.

However, a disadvantage of extra capsular cataract extraction is the high incidence of posterior lens capsule opacification, often called after-cataract, which can occur in up to 50% of cases within three years after surgery. After cataract is caused by proliferation of equatorial and anterior capsule lens epithelial cells that remain after extra capsular lens extraction. These cells proliferate to cause Sommerling rings, and along with fibroblasts that also deposit and occur on the posterior capsule, cause opacification of the posterior capsule, which interferes with vision. Prevention of after cataract would be preferable to treatment. To inhibit secondary cataract formation, the subject method provides a means for inhibiting proliferation of the remaining lens epithelial cells. For example, such cells can be induced to remain quiescent by instilling a solution containing an Hedgehog antagonist preparation into the anterior chamber of the eye after lens removal. Furthermore, the solution can be osmotically balanced to provide minimal effective dosage when instilled into the anterior chamber of the eye, thereby inhibiting subcapsular epithelial growth with some specificity. The compounds and method can also be used in the treatment of corneopathies marked by corneal epithelial cell proliferation, as for example in ocular epithelial disorders such as epithelial downgrowth or squamous cell carcinomas of the ocular surface.

Levine, et al., J. Neurosci., 1997, 17:6277 showed that Hedgehog proteins can regulate mitogenesis and photoreceptor differentiation in the vertebrate retina, and Ihh is a candidate factor from the pigmented epithelium to promote retinal progenitor proliferation and photoreceptor differentiation. Likewise, Jensen, et al., Development, 1991, 124:363 demonstrated that treatment of cultures of perinatal mouse retinal cells with the amino-terminal fragment of Sonic Hedgehog protein results in an increase in the proportion of cells that incorporate bromodeoxuridine, in total cell numbers, and in rod photoreceptors, amacrine cells and Muller Glial cells, suggesting that Sonic Hedgehog promotes the proliferation of retinal precursor cells. Thus, the compounds and method described herein can be used in the treatment of proliferative diseases of retinal cells and regulate photoreceptor differentiation.

Yet another aspect of the present invention relates to the use of the subject method to control hair growth. Hair is basically composed of keratin, a tough and insoluble protein; its chief strength lies in its disulphide bond of cystine. Each individual hair comprises a cylindrical shaft and a root, and is contained in a follicle, a flask-like depression in the skin. The bottom of the follicle contains a finger-like projection termed the papilla, which consists of connective tissue from which hair grows, and through which blood vessels supply the cells with nourishment. The shaft is the part mat extends outwards from the skin surface, whilst the root has been described as the buried part of the hair. The base of the root expands into the hair bulb, which rests upon the papilla. Cells from which the hair is produced grow in the bulb of the follicle; they are extruded in the form of fibers as the cells proliferate in the follicle. Hair “growth” refers to the formation and elongation of the hair fiber by the dividing cells.

As is well known in the art, the common hair cycle is divided into three stages: anagen, catagen and telogen. During the active phase (anagen), the epidermal stem cells of the dermal papilla divide rapidly. Daughter cells move upward and differentiate to form the concentric layers of the hair itself. The transitional stage, catagen, is marked by the cessation of mitosis of the stem cells in the follicle. The resting stage is known as telogen, where the hair is retained within the scalp for several weeks before an emerging new hair developing below it dislodges the telogen-phase shaft from its follicle. From this model it has become clear that the larger the pool of dividing stem cells that differentiate into hair cells, the more hair growth occurs. Accordingly, methods for increasing or reducing hair growth can be carried out by potentiating or inhibiting, respectively, the proliferation of these stem cells.

In certain embodiments, the subject method can be employed as a way of reducing the growth of human hair as opposed to its conventional removal by cutting, shaving, or depilation. For instance, the present method can be used in the treatment of trichosis characterized by abnormally rapid or dense growth of hair, e.g. hypertrichosis. In an exemplary embodiment, Hedgehog antagonists can be used to manage hirsutism, a disorder marked by abnormal hairiness. The subject method can also provide a process for extending the duration of depilation.

Moreover, because a Hedgehog antagonist will often be cytostatic to epithelial cells, rather than cytotoxic, Smo antagonists can be used to protect hair follicle cells from cytotoxic agents that require progression into S-phase of the cell-cycle for efficacy, e.g. radiation-induced death. Treatment by the subject method can provide protection by causing the hair follicle cells to become quiescent, e.g., by inhibiting the cells from entering S phase, and thereby preventing the follicle cells from undergoing mitotic catastrophe or programmed cell death. For instance, Hedgehog antagonists can be used for patients undergoing chemo- or radiation-therapies that ordinarily result in hair loss. By inhibiting cell-cycle progression during such therapies, the subject treatment can protect hair follicle cells from death that might otherwise result from activation of cell death programs. After the therapy has concluded, the instant method can also be removed with concommitant relief of the inhibition of follicle cell proliferation.

The subject method can also be used in the treatment of folliculitis, such as folliculitis decalvans, folliculitis ulerythematosa reticulata or keloid folliculitis. For example, a cosmetic preparation of a Smo antagonist can be applied topically in the treatment of pseudofolliculitis, a chronic disorder occurring most often in the submandibular region of the neck and associated with shaving, the characteristic lesions of which are erythematous papules and pustules containing buried hairs.

In another aspect of the invention, the subject method can be used to induce differentiation and/or inhibit proliferation of epithelially derived tissue. Such forms of these molecules can provide a basis for differentiation therapy for the treatment of hyperplastic and/or neoplastic conditions involving epithelial tissue. For example, such preparations can be used for the treatment of cutaneous diseases in which there is abnormal proliferation or growth of cells of the skin.

For instance, the methods and composition of the invention are intended for the treatment of hyperplastic epidermal conditions, such as keratosis, as well as for the treatment of neoplastic epidermal conditions such as those characterized by a high proliferation rate for various skin cancers, as for example basal cell carcinoma or squamous cell carcinoma. The subject method can also be used in the treatment of autoimmune diseases affecting the skin, in particular, of dermatological diseases involving morbid proliferation and/or keratinization of the epidermis, as for example, caused by psoriasis or atopic dermatosis.

Many common diseases of the skin, such as psoriasis, squamous cell carcinoma, keratoacanthoma and actinic keratosis are characterized by localized abnormal proliferation and growth. For example, in psoriasis, which is characterized by scaly, red, elevated plaques on the skin, the keratinocytes are known to proliferate much more rapidly than normal and to differentiate less completely.

In one embodiment, the subject method is suitable for the treatment of dermatological ailments linked to keratinization disorders causing abnormal proliferation of skin cells, which disorders may be marked by either inflammatory or non-inflammatory components. To illustrate, Smo antagonist, e.g., which promotes quiescense or differentiation can be used to treat varying forms of psoriasis, be they cutaneous, mucosal or ungual. Psoriasis, as described above, is typically characterized by epidermal keratinocytes that display marked proliferative activation and differentiation along a “regenerative” pathway. Treatment with an antiproliferative embodiment of the subject method can be used to reverse the pathological epidermal activiation and can provide a basis for sustained remission of the disease.

A variety of other keratotic lesions are also candidates for treatment with the subject method. Actinic keratoses, for example, are superficial inflammatory premalignant tumors arising on sun-exposed and irradiated skin. The lesions are erythematous to brown with variable scaling. Current therapies include excisional and cryosurgery. These treatments are painful, however, and often produce cosmetically unacceptable scarring. Accordingly, treatment of keratosis, such as actinic keratosis, can include application, preferably topical, of a Smo antagonist composition in amounts sufficient to inhibit hyperproliferation of epidermal/epidermoid cells of the lesion.

Acne represents yet another dermatologic ailment which may be treated by the subject method. Acne vulgaris, for instance, is a multifactorial disease most commonly occurring in teenagers and young adults, and is characterized by the appearance of inflammatory and noninflammatory lesions on the face and upper trunk. The basic defect which gives rise to acne vulgaris is hypercornification of the duct of a hyperactive sebaceous gland. Hypercornification blocks the normal mobility of skin and follicle microorganisms, and in so doing, stimulates the release of lipases by Propinobacterium acnes and Staphylococcus epidermidis bacteria and Pitrosporum ovale, a yeast. Treatment with an antiproliferative Smo antagonist, particularly topical preparations, may be useful for preventing the transitional features of the ducts, e.g. hypercornification, which lead to lesion formation. The subject treatment may further include, for example, antibiotics, retinoids and antiandrogens.

The present invention also provides a method for treating various forms of dermatitis. Dermatitis is a descriptive term referring to poorly demarcated lesions that are either pruritic, erythematous, scaley, blistered, weeping, fissured or crusted. These lesions arise from any of a wide variety of causes. The most common types of dermatitis are atopic, contact and diaper dermatitis. For instance, seborrheic dermatitis is a chronic, usually pruritic, dermatitis with erythema, dry, moist, or greasy scaling, and yellow crusted patches on various areas, especially the scalp, with exfoliation of an excessive amount of dry scales. The subject method can also be used in the treatment of stasis dermatitis, an often chronic, usually eczematous dermatitis. Actinic dermatitis is dermatitis that due to exposure to actinic radiation such as that from the sun, ultraviolet waves or x- or gamma-radiation. According to the present invention, the subject method can be used in the treatment and/or prevention of certain symptoms of dermatitis caused by unwanted proliferation of epithelial cells. Such therapies for these various forms of dermatitis can also include topical and systemic corticosteroids, antipuritics, and antibiotics.

For example, it is contemplated that the subject method could be used to inhibit angiogenesis. Given that Hedgehog promotes angiogenesis, Smo antagonists are expected to act as angiogenesis inhibitors, particularly in situations where some level of Hedgehog signaling is necessary for angiogenesis.

Angiogenesis is fundamental to many disorders. Persistent, unregulated angiogenesis occurs in a range of disease states, tumor metastases and abnormal growths by endothelial cells. The vasculature created as a result of angiogenic processes supports the pathological damage seen in these conditions. The diverse pathological states created due to unregulated angiogenesis have been grouped together as angiogenic dependent or angiogenic associated diseases. Therapies directed at control of the angiogenic processes could lead to the abrogation or mitigation of these diseases.

Diseases caused by, supported by or associated with angiogenesis include ocular neovascular disease, age-related macular degeneration, diabetic retinopathy, retinopathy of prematurity, corneal graft rejection, neovascular glaucoma, retrolental fibroplasia, epidemic keratoconjunctivitis, Vitamin A deficiency, contact lens overwear, atopic keratitis, superior limbic keratitis, pterygium keratitis sicca, Sjogren's, acne rosacea, phylectenulosis, syphilis, Mycobacteria infections, lipid degeneration, chemical burns, bacterial ulcers, fungal ulcers, Herpes simplex infections, Herpes zoster infections, protozoan infections, Kaposi sarcoma, Mooren ulcer, Terrien's marginal degeneration, marginal keratolysis, rheumatoid arthritis, systemic lupus, polyarteritis, trauma, Wegeners sarcoidosis, Scleritis, Stevens Johnson disease, periphigoid radial keratotomy, corneal graph rejection, rheumatoid arthritis, osteoarthritis chronic inflammation (e.g., ulcerative colitis or Crohn's disease), hemangioma, Osier-WeberRendu disease, and hereditary hemorrhagic telangiectasia.

In addition, angiogenesis plays a critical role in cancer. A tumor cannot expand without a blood supply to provide nutrients and remove cellular wastes. Tumors in which angiogenesis is important include solid tumors such as rhabdomyosarcomas, retinoblastoma, Ewing sarcoma, neuroblastoma, and osteosarcoma, and benign tumors such as acoustic neuroma, neurofibroma, trachoma and pyogenic granulomas. Angiogenic factors have been found associated with several solid tumors. Prevention of angiogenesis could halt the growth of these tumors and the resultant damage to the animal due to the presence of the tumor. Angiogenesis is also associated with blood-born tumors such as leukemias, any of various acute or chronic neoplastic diseases of the bone marrow in which unrestrained proliferation of white blood cells occurs, usually accompanied by anemia, impaired blood clotting, and enlargement of the lymph nodes, liver, and spleen. It is believed that angiogenesis plays a role in the abnormalities in the bone marrow that give rise to leukemia like tumors.

In addition to tumor growth, angiogenesis is important in metastasis. Initially, angiogenesis is important is in the vascularization of the tumor which allows cancerous cells to enter the blood stream and to circulate throughout the body. After the tumor cells have left the primary site, and have settled into the secondary, metastasis site, angiogenesis must occur before the new tumor can grow and expand. Therefore, prevention of angiogenesis could lead to the prevention of metastasis of tumors and possibly contain the neoplastic growth at the primary site.

Angiogenesis is also involved in normal physiological processes such as reproduction and wound healing. Angiogenesis is an important step in ovulation and also in implantation of the blastula after fertilization. Prevention of angiogenesis could be used to induce amenorrhea, to block ovulation or to prevent implantation by the blastula.

The subject method can also be useful for the treatment and/or prevention of respiratory distress syndrome or other disorders resulting from inappropriate lung surface tension. Respiratory distress syndrome results from insufficient surfactant in the alveolae of the lungs. The lungs of vertebrates contain surfactant, a complex mixture of lipids and protein that causes surface tension to rise during lung inflation and decrease during lung deflation. During lung deflation, surfactant decreases such that there are no surface forces that would otherwise promote alveolar collapse. Aerated alveoli that have not collapsed during expiration permit continuous oxygen and carbon dioxide transport between blood and alveolar gas and require much less force to inflate during the subsequent inspiration. During inflation, lung surfactant increases surface tension as the alveolar surface area increases. A rising surface tension in expanding alveoli opposes over-inflation in those airspaces and tends to divert inspired an to less well-aerated alveoli, thereby facilitating even lung aeration.

Respiratory distress syndrome is particularly prevalent among premature infants. Lung surfactant is normally synthesized at a very low rate until the last six weeks of fetal life. Human infants born more than six weeks before the normal term of a pregnancy have a high risk of being born with inadequate amounts of lung surfactant and inadequate rates of surfactant synthesis. The more prematurely an infant is born, the more severe the surfactant deficiency is likely to be. Severe surfactant deficiency can lead to respiratory failure within a few minutes or hours of birth. The surfactant deficiency produces progressive collapse of alveoli (atelectasis) because of the decreasing ability of the lung to expand despite maximum inspiratory effort. As a result, inadequate amounts of oxygen reach the infant's blood. RDS can occur in adults as well, typically as a consequence of failure in surfactant biosynthesis.

Lung tissue of premature infants shows high activity of the Hedgehog signaling pathway. Inhibition of this pathway using Hedgehog antagonists increases the formation of lamellar bodies and increases the expression of genes involved in surfactant biosynthesis. Lamellar bodies are subcellular structures associated with surfactant biosynthesis. For these reasons, treatment of premature infants with a Hedgehog antagonist should stimulate surfactant biosynthesis and ameliorate RDS. In cases where adult RDS is associated with Hedgehog pathway activation, treatment with Hedgehog antagonists should also be effective.

It is further contemplated that the use of Smo antagonists may be specifically targeted to disorders where the affected tissue and/or cells evince high Hedgehog pathway activation. Expression of Gli genes is activated by the Hedgehog signaling pathway, including Gli-1, Gli-2 and Gli-3. Gli-1 expression is most consistently correlated with Hedgehog signaling activity across a wide range of tissues and disorders, while Gli-3 is somewhat less so. The Gli genes encode transcription factors that activate expression of many genes needed to elicit the full effects of Hedgehog signaling. However, the Gli-3 transcription factor can also act as a repressor of Hedgehog effector genes, and therefore, expression of Gli-3 can cause a decreased effect of the Hedgehog signaling pathway. Whether Gli-3 acts as a transcriptional activator or repressor depends on post-translational events, and therefore it is expected that methods for detecting the activating form (versus the repressing form) of Gli-3 protein would also be a reliable measure of Hedgehog pathway activation, Gli-2 gene expression is expected to provide a reliable marker for Hedgehog pathway activation. The Gli-1 gene is strongly expressed in a wide array of cancers, hyperplasias and immature lungs, and serves as a marker for the relative activation of the Hedgehog pathway. In addition, tissues, such as immature lung, that have high Gli gene expression are strongly affected by Hedgehog inhibitors. Accordingly, it is contemplated that the detection of Gli gene expression may be used as a powerful predictive tool to identify tissues and disorders that will particularly benefit from treatment with a Hedgehog antagonist.

In preferred embodiments, Gli-1 expression levels are detected, either by direct detection of the transcript or by detection of protein levels or activity. Transcripts may be detected using any of a wide range of techniques that depend primarily on hybridization of probes to the Gli-1 transcripts or to cDNAs synthesized therefrom. Well known techniques include Northern blotting, reverse-transcriptase PCR and microarray analysis of transcript levels. Methods for detecting Gli protein levels include Western blotting, immunoprecipitation, two-dimensional polyacrylamide gel electrophoresis (2D SDS-PAGE) (preferably compared against a standard wherein the position of the Gli proteins has been determined), and mass spectroscopy. Mass spectroscopy may be coupled with a series of purification steps to allow highthroughput identification of many different protein levels in a particular sample. Mass spectroscopy and 2D SDS-PAGE can also be used to identity post-transcriptional modifications to proteins including proteolytic events, ubiquitination, phosphorylation, lipid modification etc. Gli activity may also be assessed by analyzing binding to substrate DNA or in vitro transcriptional activation of target promoters. Gel shift assays, DNA footprinting assays and DNA-protein crosslinking assays are all methods that may be used to assess the presence of a protein capable of binding to Gli binding sites on DNA.

In some embodiments, Gli transcript levels are measured and diseased or disordered tissues showing abnormally high Gli levels are treated with a Hedgehog antagonist Premature lung tissue, lung cancers (e.g., adenocarcinomas, broncho-alveolar adenocarcinomas, small cell carcinomas), breast cancers (e.g., inferior ductal carcinomas, inferior lobular carcinomas, tubular carcinomas), prostate cancers (e.g., adenocarcinomas), and benign prostatic hyperplasias all show strongly elevated Gli-1 expression levels in certain cases. Accordingly, Gli-1 expression levels are a powerful diagnostic device to determine which of these tissues should be treated with a Hedgehog antagonist. In addition, mere is substantial correlative evidence that cancers of urothelial cells (e.g., bladder cancer, other urogenital cancers) will also have elevated Gli-1 levels in certain cases. For example, it is known that loss of heterozygosity on chromosome 9q22 is common in bladder cancers. The Ptch1 gene is located at this position and Ptch1 loss of function is probably a partial cause of hyperproliferation, as in many other cancer types. Accordingly, such cancers would also show high Gli expression and would be particularly amenable to treatment with a Hedgehog antagonist.

Expression of Ptch1 and Ptch2 is also activated by the Hedgehog signaling pathway, but these genes are inferior to the Gli genes as markers of Hedgehog pathway activation. In certain tissues only one of Ptch1 or Ptch2 is expressed although the Hedgehog pathway is highly active. For example, in testicular development, desert Hedgehog plays an important role and the Hedgehog pathway is activated. Accordingly, these genes are individually unreliable as markers for Hedgehog pathway activation, although simultaneous measurement of both genes can be a useful indicator for tissues to be treated with a Smo antagonist.

The compounds and method described herein can be used as part of process for generating and/or maintaining an array of different vertebrate tissue both in vitro and in vivo because of the apparently broad involvement of Hedgehog, Ptch1, and smoothened in the formation of ordered spatial arrangements of differentiated tissues in vertebrates. As used herein, the term “proliferating” and “proliferation” refer to cells undergoing mitosis.

For example, the method is applicable to cell culture techniques wherein it is desirable to modulate the level of Hedgehog signaling. In vitro neuronal culture systems have proved to be fundamental and indispensable tools for the study of neural development, as well as the identification of neurotrophic factors such as nerve growth factor (NGF), ciliary trophic factors (CNTF), and brain derived neurotrophic factor (BDNF).

Accordingly, the method described herein can be in used cultures of neuronal stem cells, such as in the use of such cultures for the generation of new neurons and glia. In such embodiments of the subject method, the cultured cells can be contacted with a compound described herein in order to alter the rate of proliferation of neuronal stem cells in the culture and/or alter the rate of differentiation, or to maintain the integrity of a culture of certain terminally differentiated neuronal cells. In some embodiments, the subject method can be used to culture, for example, sensory neurons or, alternatively, motor neurons. Such neuronal cultures can be used as convenient assay systems as well as sources of implantable cells for therapeutic treatments.

To further illustrate other uses of the compounds and method described herein, it is noted that intracerebral grafting has emerged as an additional approach to central nervous system therapies. For example, one approach to repairing damaged brain tissues involves the transplantation of cells from fetal or neonatal animals into the adult brain. See for example Dunnett, et al., J. Exp. Biol., 1987, 123:265-289 and Freund, et al., J. Neurosci., 1985, 5:603-616, content of both which is incorporated herein by reference in its entirety. Fetal neurons from a variety of brain regions can be successfully incorporated into the adult brain, and such grafts can alleviate behavioral defects. For example, movement disorder induced by lesions of dopaminergic projections to the basal ganglia can be prevented by grafts of embryonic dopaminergic neurons. Complex cognitive functions that are impaired after lesions of the neocortex can also be partially restored by grafts of embryonic cortical cells. The method described herein can be used to regulate the growth state in the culture, or where fetal tissue is used, especially neuronal stem cells, can be used to regulate the rate of differentiation of the stem cells.

Stem cells useful in the present invention are generally known. For example, several neural crest cells have been identified, some of which are multipotent and likely represent uncommitted neural crest cells, and others of which can generate only one type of cell, such as sensory neurons, and likely represent committed progenitor cells. The role of compounds and method described herein to culture such stem cells can be to regulate differentiation of the uncommitted progenitor, or to regulate further restriction of the developmental fate of a committed progenitor cell towards becoming a terminally differentiated neuronal cell. For example, the compounds and method described herein can be used in vitro to regulate the differentiation of neural crest cells into glial cells, schwann cells, chromaffin cells, cholinergic sympathetic or parasympathetic neurons, as well as peptidergic and serotonergic neurons. The compounds described herein can be used alone, or can be used in combination with other neurotrophic factors that act to more particularly enhance a particular differentiation fate of the neuronal progenitor cell.

In addition to the implantation of cells cultured with the compounds and method described herein, yet another aspect of the present invention concerns the therapeutic application of the compounds to regulate the growth state of neurons and other neuronal cells in both the central nervous system and the peripheral nervous system. The ability of Ptch1, Hedgehog, and smoothened to regulate neuronal differentiation during development of the nervous system and also presumably in the adult state indicates that, in certain instances, the compounds and method described herein can be expected to facilitate control of adult neurons with regard to maintenance, functional performance, and aging of normal cells; repair and regeneration processes in chemically or mechanically lesioned cells; and treatment of degeneration in certain pathological conditions. Accordingly, in some embodiments, the compounds and method described herein can be used of for treatment, prevention and/or reduction of severity of neurological conditions deriving from: (i) acute, subacute, or chronic injury to the nervous system, including traumatic injury, chemical injury, vascular injury and deficits (such as the ischemia resulting from stroke), together with infectious/inflammatory and tumor-induced injury; (ii) aging of the nervous system including Alzheimer's disease; (iii) chronic neurodegenerative diseases of the nervous system, including Parkinson's disease, Huntington's chorea, amyotrophic lateral sclerosis and the like, as well as spinocerebellar degenerations; and (iv) chronic immunological diseases of the nervous system or affecting the nervous system, including multiple sclerosis.

Without wishing to be bound by a theory, the compounds and method described herein can also be used in generating nerve prostheses for the repair of central and peripheral nerve damage. In particular, where a crushed or severed axon is intubulated by use of a prosthetic device, Hedgehog antagonists can be added to the prosthetic device to regulate the rate of growth and regeneration of the dendritic processes. Exemplary nerve guidance channels are described in U.S. Pat. No. 5,092,871 and No. 4,955,892, content of both of which is incorporate herein by reference in its entirety.

In some embodiments, the compounds and method described herein can be used in the treatment of neoplastic or hyperplastic transformations such as may occur in the central nervous system. For instance, the compounds and method can be utilized to cause such transformed cells to become either post-mitotic or apoptotic. The compounds and method can, therefore, be used as part of a treatment for, e.g., malignant Gliomas, meningiomas, medulloblastomas, neuroectodermal tumors, and ependymomas. In some embodiments, the compounds and method can be used as part of a treatment regimen for malignant medulloblastoma and other primary CNS malignant neuroectodermal tumors.

Yet another aspect of the present invention concerns the observation in the art that It is well known that Ptch1, Hedgehog, and/or smoothened are involved in morphogenic signals involved in other vertebrate organogenic pathways in addition to neuronal differentiation as described above, having apparent roles in other endodermal patterning, as well as both mesodermal and endodermal differentiation processes. Accordingly, the compounds and method describe herein can also be utilized for both cell culture and therapeutic methods involving generation and maintenance of non-neuronal tissue.

Ptch1, Hedgehog, and smoothened are also apparently involved in controlling the development of stem cells responsible for formation of the digestive tract, liver, lungs, and other organs which derive from the primitive gut. Shh serves as an inductive signal from the endoderm to the mesoderm, which is critical to gut morphogenesis. Therefore, the compounds and method described herein can be employed for regulating the development and maintenance of an artificial liver that can have multiple metabolic functions of a normal liver. In some embodiments, the compounds and method described herein can be used to regulate the proliferation and differentiation of digestive tube stem cells to form hepatocyte cultures which can be used to populate extracellular matrices, or which can be encapsulated in biocompatible polymers, to form both implantable and extracorporeal artificial livers. Additionally, pharmaceutically acceptable compositions of the compounds described herein can be used in conjunction with transplantation of such artificial livers, as well as embryonic liver structures, to regulate uptake of intraperitoneal implantation, vascularization, and in vivo differentiation and maintenance of the engrafted liver tissue.

In yet other embodiments, the compounds described herein can be employed therapeutically to regulate such organs after physical, chemical or pathological insult. For instance, therapeutic compositions comprising can be utilized in liver repair subsequent to a partial hepatectomy.

The generation of the pancreas and small intestine from the embryonic gut depends on intercellular signaling between the endodermal and mesodermal cells of the gut. In particular, the differentiation of intestinal mesoderm into smooth muscle has been suggested to depend on signals from adjacent endodermal cells. One candidate mediator of endodermally derived signals in the embryonic hindgut is Sonic Hedgehog. See, for example, Apelqvist, et al., Curr. Biol., 1997, 7:801-804. The Shh gene is expressed throughout the embryonic gut endoderm with the exception of the pancreatic bud endoderm, which instead expresses high levels of the homeodomain protein Ipfl/Pdxl (insulin promoter factor 1/pancreatic and duodenal homeobox 1), an essential regulator of early pancreatic development. This differential expression of endodermally derived Shh controls the fate of adjacent mesoderm at different regions of the gut tube. Accordingly, the compounds and method described herein can be used to control or regulate the proliferation and/or differentiation of pancreatic tissue both in vivo and in vitro.

There are a wide variety of pathological cell proliferative and differentiative conditions for which the compounds and method described herein can provide therapeutic benefits, with the general strategy being, for example, the correction of aberrant insulin expression, or modulation of differentiation. Thus, the compounds and method described herein can be used for inducing and/or maintaining a differentiated state, enhancing survival and/or affecting proliferation of pancreatic cells. For example, the compounds described herein can be used as part of a process to generate and/or maintain such tissue both in vitro and in vivo. For instance, modulation of the Hedgehog signaling patway can be employed in both cell culture and therapeutic methods involving generation and maintenance of beta-cells and possibly also for non-pancreatic tissue, such as in controlling the development and maintenance of tissue from the digestive tract, spleen, lungs, urogenital organs (e.g., bladder), and other organs which derive from the primitive gut.

The compounds and method described herein can be used in the treatment of hyperplastic and neoplastic disorders effecting pancreatic tissue, particularly those characterized by aberrant proliferation of pancreatic cells. For instance, pancreatic cancers are marked by abnormal proliferation of pancreatic cells, which can result in alterations of insulin secretory capacity of the pancreas. For instance, certain pancreatic hyperplasias, such as pancreatic carcinomas, can result in hypoinsulinemia due to dysfunction of beta-cells or decreased islet cell mass.

Moreover, manipulation of Hedgehog signaling pathway at different points can be useful as part of a strategy for reshaping/repairing pancreatic tissue both in vivo and in vitro.

Accordingly, the compounds described herein can be employed therapeutically to regulate the pancreas after physical, chemical or pathological insult. Additionally, the method described herein can be applied to cell culture techniques, and in particular, can be employed to enhance the initial generation of prosthetic pancreatic tissue devices. Manipulation of proliferation and differentiation of pancreatic tissue, for example, by altering Hedgehog signaling can provide a means for more carefully controlling the characteristics of a cultured tissue. In an exemplary embodiment, a method described herein can be used to augment production of prosthetic devices which require beta-islet cells, such as may be used in the encapsulation devices described, for example, in U.S. Pat. No. 4,892,538; No. 5,106,627; No. 4,391,909; and No. 4,353,888, content of all of which is incorporated herein by reference in its entirety. Early progenitor cells to the pancreatic islets are multipotential, and apparently coactivate all the islet-specific genes from the time they first appear. As development proceeds, expression of islet-specific hormones, such as insulin, becomes restricted to the pattern of expression characteristic of mature islet cells. The phenotype of mature islet cells, however, is not stable in culture, as reappearance of embryonal traits in mature beta-cells can be observed. By utilizing the compounds and method described herein, the differentiation path or proliferative index of the cells can be regulated. Furthermore, manipulation of the differentiative state of pancreatic tissue can be utilized in conjunction with transplantation of artificial pancreas. For instance, manipulation of Hedgehog singaling to affect tissue differentiation can be utilized as a means of maintaining graft viability.

Bellusci, et al., Development, 1997, 124:53 report that Sonic Hedgehog regulates lung mesenchymal cell proliferation in vivo. Accordingly, the compounds and method described herein can be used to regulate regeneration of lung tissue, e.g., in the treatment of emphysema.

The compounds described herein can also be used in the in vitro generation of skeletal tissue, such as from skeletogenic stem cells, as well as the in vivo treatment of skeletal tissue deficiencies. For example, the compounds described herein can be used to regulate the rate of chondrogenesis and/or osteogenesis. By “skeletal tissue deficiency”, it is meant a deficiency in bone or other skeletal connective tissue at any site where it is desired to restore the bone or connective tissue, no matter how the deficiency originated, e.g., whether as a result of surgical intervention, removal of tumor, ulceration, implant, fracture, or other traumatic or degenerative conditions.

For example, the compounds and method described herein can be used as part of a regimen for restoring cartilage function to a connective tissue. Such methods are useful in, for example, the repair of defects or lesions in cartilage tissue which is the result of degenerative wear such as that which results in arthritis, as well as other mechanical derangements which may be caused by trauma to the tissue, such as a displacement of torn meniscus tissue, meniscectomy, a Taxation of a joint by a torn ligament, malignment of joints, bone fracture, or by hereditary disease.

The compounds and method are also useful for remodeling cartilage matrix, such as in plastic or reconstructive surgery, as well as periodontal surgery. The compounds and method can also be applied to improving a previous reparative procedure, for example, following surgical repair of a meniscus, ligament, or cartilage. Furthermore, it can prevent the onset or exacerbation of degenerative disease if applied early enough after trauma.

In some embodiments, the method comprises treating the afflicted connective tissue with a therapeutically sufficient amount of a compound described hereinto regulate a cartilage repair response in the connective tissue by managing the rate of differentiation and/or proliferation of chondrocytes embedded in the tissue. Such connective tissues as articular cartilage, interarticular cartilage (menisci), costal cartilage (connecting the true ribs and the sternum), ligaments, and tendons are particularly amenable to treatment in reconstructive and/or regenerative therapies using the subject method. As used herein, regenerative therapies include treatment of degenerative states which have progressed to the point of which impairment of the tissue is obviously manifest, as well as preventive treatments of tissue where degeneration is in its earliest stages or imminent.

The compounds and method described herein can be used as part of a therapeutic intervention in the treatment of cartilage of a diarthroidal joint, such as a knee, an ankle, an elbow, a hip, a wrist, a knuckle of either a finger or toe, or a tempomandibular joint. The treatment can be directed to the meniscus of the joint, to the articular cartilage of the joint, or both. To further illustrate, the compounds and method can be used to treat a degenerative disorder of a knee, such as which might be the result of traumatic injury (e.g., a sports injury or excessive wear) or osteoarthritis. The compounds can be administered as an injection into the joint with, for instance, an arthroscopic needle. In some instances, the injected agent can be in the form of a hydrogel or other slow release vehicle described above in order to permit a more extended and regular contact of the agent with the treated tissue.

The compounds and method described herein can used in the field of cartilage transplantation and prosthetic device therapies. However, problems arise, for instance, because the characteristics of cartilage and fibrocartilage vary between different tissue: such as between articular, meniscal cartilage, ligaments, and tendons, between the two ends of the same ligament or tendon, and between the superficial and deep parts of the tissue. The zonal arrangement of these tissues may reflect a gradual change in mechanical properties, and failure occurs when implanted tissue, which has not differentiated under those conditions, lacks the ability to appropriately respond. For instance, when meniscal cartilage is used to repair anterior cruciate ligaments, the tissue undergoes a metaplasia to pure fibrous tissue. By regulating the rate of chondrogenesis, the compounds and methods can be used to particularly address this problem, by helping to adaptively control the implanted cells in the new environment and effectively resemble hypertrophic chondrocytes of an earlier developmental stage of the tissue.

In similar fashion, the compounds and method described herein can be applied to enhancing both the generation of prosthetic cartilage devices and to their implantation. The need for improved treatment has motivated research aimed at creating new cartilage that is based on collagen-glycosaminoglycan templates, isolated chondrocytes, and chondrocytes attached to natural or synthetic polymers. See, for example, Stone, et al., Clin. Orthop. Relat. Red., 1997, 252:129; Grande, et al., J. Orthop. Res., 1989, 7:208; Takigawa, et al., Bone Miner., 1987, 2:449; Walitani, et al., J. Bone Jt. Surg., 1989, 71B:74; Vacanti, et al., Plast Reconstr. Surg., 1991, 88:753; von Schroede, r et al., J. Biomed. Mater. Res., 1991, 25:329; and Freed, et al., J. Biomed. Mater. Res., 1993, 27:11, U.S. Pat. No. 5,041,138, content of all of which is incorporated herein by reference in its entirety. For example, chondrocytes can be grown in culture on biodegradable, biocompatible highly porous scaffolds formed from polymers such as polyglycolic acid, polylactic acid, agarose gel, or other polymers that degrade over time as function of hydrolysis of the polymer backbone into innocuous monomers. The matrices are designed to allow adequate nutrient and gas exchange to the cells until engraftment occurs. The cells can be cultured in vitro until adequate cell volume and density has developed for the cells to be implanted. One advantage of the matrices is that they can be cast or molded into a desired shape on an individual basis, so that the final product closely resembles the patient's own ear or nose (by way of example), or flexible matrices can be used which allow for manipulation at the time of implantation, as in a joint. The implants can be contacted with a compound described herein during certain stages of the culturing process in order to manage the rate of differentiation of chondrocytes and the formation of hypertrophic chrondrocytes in the culture.

The implanted device can be treated with a compound described herein in order to actively remodel the implanted matrix and to make it more suitable for its intended function. As set out above with respect to tissue transplants, the artificial transplants suffer from the same deficiency of not being derived in a setting which is comparable to the actual mechanical environment in which the matrix is implanted. The ability to regulate the chondrocytes in the matrix by the compounds and method described herein can allow the implant to acquire characteristics similar to the tissue for which it is intended to replace.

The compounds and method of the invention can be used to enhance attachment of prosthetic devices. To illustrate, the compounds and method can be used in the implantation of a periodontal prosthesis, wherein the treatment of the surrounding connective tissue stimulates formation of periodontal ligament about the prosthesis.

The compounds and method described herein can be employed as part of a regimen for the generation of bone (osteogenesis) at a site in the animal where such skeletal tissue is deficient. Indian Hedgehog is particularly associated with the hypertrophic chondrocytes that are ultimately replaced by osteoblasts. For instance, administration of a compound described herein can be employed as part of a method for regulating the rate of bone loss in a subject, for example, to control endochondral ossification in the formation of a “model” for ossification.

The compounds described herein can be used to regulate spermatogenesis. The Hedgehog proteins, particularly Dhh, have been shown to be involved in the differentiation and/or proliferation and maintenance of testicular germ cells. Dhh expression is initiated in Sertoli cell precursors shortly after the activation of Sry (testicular determining gene) and persists in the testis into the adult. Males are viable but infertile, owing to a complete absence of mature sperm. Examination of the developing testis in different genetic backgrounds suggests that Dhh regulates both early and late stages of spermatogenesis. See, for example, Bitgood, et al., Curr. Biol., 1996, 6:298. Accordingly, a compound described herein can be used as a contraceptive. In similar fashion, the compounds described herein can be used for modulating normal ovarian function.

As used herein, a “subject” means a human or animal. Examples of subjects include primates (e.g., humans, and monkeys). Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments of the aspects described herein, the subject is a mammal, e.g., a primate, e.g., a human. The terms, “patient” and “subject” are used interchangeably herein. The terms, “patient” and “subject” are used interchangeably herein. A subject can be male or female. A subject can be one who has not been previously diagnosed with inflammation, inflammatory disease or condition, and/or cancer.

Preferably, the subject is a mammal. The mammal can be a human, non-human primate, mouse, rat, dog, cat, horse, or cow, but are not limited to these examples. Mammals other than humans can be advantageously used as subjects that represent animal models of inflammatory disease or disorder, or as animal models of cancer. In addition, the methods and compositions described herein can be used to treat domesticated animals and/or pets.

A subject can be one who has been previously diagnosed with cancer and/or having a precancerous condition. Without wishing to be bound by a theory, a subject can be diagnosed as having cancer and/or a precancerous condition.

A method described herein can further comprise selecting a subject who has cancer and/or a precancerous condition. The method can also comprise the step of diagnosing a subject for cancer and/or a precancerous condition before onset of administration or treatment regime.

For administration to a subject, a compound described herein can be provided in pharmaceutically acceptable compositions. Accordingly, in one aspect, the invention provides a pharmaceutical composition comprising one or more of a compound described herein, formulated together with one or more pharmaceutically acceptable carriers (additives) and/or diluents. As described in detail below, the pharmaceutical compositions of the present invention can be specially formulated for administration in solid or liquid form, including those adapted for the following: (1) oral administration, for example, drenches (aqueous or non-aqueous solutions or suspensions), gavages, lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally; or (9) nasally. Additionally, compounds can be implanted into a patient or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960, content of all of which is herein incorporated by reference.

As used here, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material. A pharmaceutically acceptable carrier will not promote the raising of an immune response to an agent with which it is admixed, unless so desired. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alchols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

The amount of a compound described herein that can be combined with a carrier material to produce a single dosage form will generally be that amount of the compound that produces a therapeutic effect. Generally out of one hundred percent, this amount will range from about 0.001% to 99% of the compound, preferably from about 0.01% to about 70%, most preferably from 5% to about 30%.

Toxicity and therapeutic efficacy can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compositions that exhibit large therapeutic indices, are preferred.

As used herein, the term ED denotes effective dose and is used in connection with animal models. The term EC denotes effective concentration and is used in connection with in vitro models.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized.

The phrase “therapeutically-effective amount” as used herein means that amount of a compound, material, or composition comprising a conjugate which is effective for producing some desired therapeutic effect in at least a sub-population of cells in an animal at a reasonable benefit/risk ratio applicable to any medical treatment.

Determination of a therapeutically effective amount is well within the capability of those skilled in the art. Generally, a therapeutically effective amount can vary with the subject's history, age, condition, sex, as well as the severity and type of the medical condition in the subject, and administration of other pharmaceutically active agents.

The therapeutically effective dose can be estimated initially from cell culture assays. A dose may be formulated in animal models to achieve a circulating plasma concentration range that includes the IC₅₀ (i.e., the concentration of the therapeutic which achieves a half-maximal inhibition of symptoms) as determined in cell culture. Levels in plasma may be measured, for example, by high performance liquid chromatography. The effects of any particular dosage can be monitored by a suitable bioassay.

The dosage may be determined by a physician and adjusted, as necessary, to suit observed effects of the treatment. Generally, the compositions are administered so that a compound or composition described herein is given at a dose from 1 μg/kg to 150 mg/kg, 1 μg/kg to 100 mg/kg, 1 μg/kg to 50 mg/kg, 1 μg/kg to 20 mg/kg, 1 μg/kg to 10 mg/kg, 1 μg/kg to 1 mg/kg, 100 μg/kg to 100 mg/kg, 100 μg/kg to 50 mg/kg, 100 μg/kg to 20 mg/kg, 100 μg/kg to 10 mg/kg, 100 μg/kg to 1 mg/kg, 1 mg/kg to 100 mg/kg, 1 mg/kg to 50 mg/kg, 1 mg/kg to 20 mg/kg, 1 mg/kg to 10 mg/kg, 10 mg/kg to 100 mg/kg, 10 mg/kg to 50 mg/kg, or 10 mg/kg to 20 mg/kg. It is to be understood that ranges given here include all intermediate ranges, for example, the range 1 mg/kg to 10 mg/kg includes 1 mg/kg to 2 mg/kg, 1 mg/kg to 3 mg/kg, 1 mg/kg to 4 mg/kg, 1 mg/kg to 5 mg/kg, 1 mg/kg to 6 mg/kg, 1 mg/kg to 7 mg/kg, 1 mg/kg to 8 mg/kg, 1 mg/kg to 9 mg/kg, 2 mg/kg to 10 mg/kg, 3 mg/kg to 10 mg/kg, 4 mg/kg to 10 mg/kg, 5 mg/kg to 10 mg/kg, 6 mg/kg to 10 mg/kg, 7 mg/kg to 10 mg/kg, 8 mg/kg to 10 mg/kg, 9 mg/kg to 10 mg/kg, and the like. It is to be further understood that the ranges intermediate to the given above are also within the scope of this invention, for example, in the range 1 mg/kg to 10 mg/kg, dose ranges such as 2 mg/kg to 8 mg/kg, 3 mg/kg to 7 mg/kg, 4 mg/kg to 6 mg/kg, and the like.

With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment or make other alteration to treatment regimen. The dosing schedule can vary from once a week to daily depending on a number of clinical factors, such as the subject's sensitivity to the compounds described herein. The desired dose can be administered everyday or every third, fourth, fifth, or sixth day. The desired dose can be administered at one time or divided into subdoses, e.g., 2-4 subdoses and administered over a period of time, e.g., at appropriate intervals through the day or other appropriate schedule. Such sub-doses can be administered as unit dosage forms. In some embodiments of the aspects described herein, administration is chronic, e.g., one or more doses daily over a period of weeks or months. Examples of dosing schedules are administration daily, twice daily, three times daily or four or more times daily over a period of 1 week, 2 weeks, 3 weeks, 4 weeks, 1 month, 2 months, 3 months, 4 months, 5 months, or 6 months or more.

As used herein, the term “administer” refers to the placement of a composition into a subject by a method or route which results in at least partial localization of the composition at a desired site such that desired effect is produced. Routes of administration suitable for the methods of the invention include both local and systemic administration. Generally, local administration results in more of the composition being delivered to a specific location as compared to the entire body of the subject, whereas, systemic administration results in delivery to essentially the entire body of the subject.

A compound described herein can be administered by any appropriate route known in the art including, but not limited to, oral or parenteral routes, including intravenous, intramuscular, subcutaneous, transdermal, airway (aerosol), pulmonary, nasal, rectal, vaginal, and topical (including on the skin, and body cavities, such as buccal, vaginal, rectal and sublingual) administration.

Exemplary modes of administration include, but are not limited to, injection, infusion, instillation, inhalation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In some embodiments of the aspects described herein, the compositions are administered by intravenous infusion or injection. In some embodiments, administration is oral.

The compounds described herein can be administrated to a subject in combination with one or more pharmaceutically active agents. Exemplary pharmaceutically active compound include, but are not limited to, those found in Harrison's Principles of Internal Medicine, 13^{th} Edition, Eds. T. R. Harrison et al. McGraw-Hill N.Y., N.Y.; Physicians Desk Reference, 50^{th} Edition, 1997, Oradell N.J., Medical Economics Co.; Pharmacological Basis of Therapeutics, 8^{th} Edition, Goodman and Gilman, 1990; United States Pharmacopeia, The National Formulary, USP XII NF XVII, 1990; current edition of Goodman and Oilman's The Pharmacological Basis of Therapeutics; and current edition of The Merck Index, the complete content of all of which are herein incorporated in its entirety.

The compound and the pharmaceutically active agent can be administrated to the subject in the same pharmaceutical composition or in different pharmaceutical compositions (at the same time or at different times). When administrated at different times, they can be administered within 5 minutes, 10 minutes, 20 minutes, 60 minutes, 2 hours, 3 hours, 4, hours, 8 hours, 12 hours, 24 hours of administration of the other When administered in different pharmaceutical compositions, routes of administration can be different.

In some embodiments, the pharmaceutically active agent is an anti-cancer compound or agent. As used herein, the term “anti-cancer compound” or “anti-cancer agent” is used to describe any compound (including its analogs, derivatives, prodrugs and pharmaceutically salts) which may be used to treat cancer. Anti-cancer compounds for use in the present invention include, but are not limited to, inhibitors of topoisomerase I and II, alkylating agents, microtubule inhibitors (e.g., taxol), and angiogenesis inhibitors. Exemplary anti-cancer compounds include, but are not limited to, paclitaxel (taxol); docetaxel; germicitibine; Aldesleukin; Alemtuzumab; alitretinoin; allopurinol; altretamine; amifostine; anastrozole; arsenic trioxide; Asparaginase; BCG Live; bexarotene capsules; bexarotene gel; bleomycin; busulfan intravenous; busulfanoral; calusterone; capecitabine; carboplatin; carmustine; carmustine with Polifeprosan Implant; celecoxib; chlorambucil; cisplatin; cladribine; cyclophosphamide; cytarabine; cytarabine liposomal; dacarbazine; dactinomycin; actinomycin D; Darbepoetin alfa; daunorubicin liposomal; daunorubicin, daunomycin; Denileukin diftitox, dexrazoxane; docetaxel; doxorubicin; doxorubicin liposomal; Dromostanolone propionate; Elliott's B Solution; epirubicin; Epoetin alfa estramustine; etoposide phosphate; etoposide (VP-16); exemestane; Filgrastim; floxuridine (intraarterial); fludarabine; fluorouracil (5-FU); fulvestrant; gemtuzumab ozogamicin; goserelin acetate; hydroxyurea; Ibritumomab Tiuxetan; idarubicin; ifosfamide; imatinib mesylate; Interferon alfa-2a; Interferon alfa-2b; irinotecan; letrozole; leucovorin; levamisole; lomustine (CCNU); mechlorethamine (nitrogenmustard); megestrol acetate; melphalan (L-PAM); mercaptopurine (6-MP); mesna; methotrexate; methoxsalen; mitomycin C; mitotane; mitoxantrone; nandrolone phenpropionate; Nofetumomab; LOddC; Oprelvekin; oxaliplatin; pamidronate; pegademase; Pegaspargase; Pegfilgrastim; pentostatin; pipobroman; plicamycin; mithramycin; porfimer sodium; procarbazine; quinacrine; Rasburicase; Rituximab; Sargramostim; streptozocin; talbuvidine (LDT); talc; tamoxifen; temozolomide; teniposide (VM-26); testolactone; thioguanine (6-TG); thiotepa; topotecan; toremifene; Tositumomab; Trastuzumab; tretinoin (ATRA); Uracil Mustard; valrubicin; valtorcitabine (monoval LDC); vinblastine; vinorelbine; zoledronate; and any mixtures thereof. In some embodiments, the anti-cancer agent is a paclitaxel-carbohydrate conjugate, e.g., a paclitaxel-glucose conjugate, as described in U.S. Pat. No. 6,218,367, content of which is herein incorporated by reference in its entirety. Some exemplary paclitaxel-carbohydrate conjugates include, but are not limited to, 2′-(GABA-succinoyl)paclitaxel, 2′-(glucose-GABA-succinoyl)paclitaxel, 2′-(glucose-succinoyl)paclitaxel, 2′-(glucose-glutamyl)paclitaxel, 2′-(glucosamide-GABA-succinoyl)paclitaxel, 2′-(glucoseamide-succinoyl)paclitaxel, 2′-(glucoseamide-glutamyl)paclitaxel, 7-(GABA-succinoyl)paclitaxel, 7-(glucose-GABA-succinoyl)paclitaxel, 7-(glucose-succinoyl)paclitaxel, 7-(glucose-glutamyl)paclitaxel, 7-(glucosamide-GABA-succinoyl)paclitaxel, 7-(glucoseamide-succinoyl)paclitaxel, and 7-(glucoseamide-glutamyl)paclitaxel.

### SELECTED DEFINITIONS

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

Other than in the operating examples, or where otherwise indicated, all numbers expressing quantities of ingredients or reaction conditions used herein should be understood as modified in all instances by the term “about.” The term “about” when used in connection with percentages may mean ±1%. Furthermore, the term “about” can mean within ±1% of a value.

The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description.

Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

The terms “decrease”, “reduced”, “reduction”, “decrease” or “inhibit” are all used herein generally to mean a decrease by a statistically significant amount. However, for avoidance of doubt, ““reduced”, “reduction” or “decrease” or “inhibit” means a decrease by at least 10% as compared to a reference level, for example a decrease by at least 10%, or at least about 20%, or at least about 30%, or at least about 40%, or at least about 50%, or at least about 60%, or at least about 70%, or at least about 80%, or at least about 90% or up to and including a 100% decrease (e.g. absent level as compared to a reference sample), or any decrease between 10-100% as compared to a reference level.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “statistically significant” or “significantly” refers to statistical significance and generally means a two standard deviation (2SD) above or below a reference level. The term refers to statistical evidence that there is a difference. It is defined as the probability of making a decision to reject the null hypothesis when the null hypothesis actually true. The decision is often made using the p-value.

The phrase “aberrant modification or mutation” of a gene refers to such genetic lesions as, for example, deletions, substitution or addition of nucleotides to a gene, as well as gross chromosomal rearrangements of the gene and/or abnormal methylation of the gene. Likewise, mis-expression of a gene refers to aberrant levels of transcription of the gene relative to those levels in a normal cell under similar conditions, as well as non-wild-type splicing of mRNA transcribed from the gene.

As used herein, the term “anti-cancer activity” or “anti-cancer properties” refers to the inhibition (in part or in whole) or prevention of unregulated cell growth and/or the inhibition (in part or in whole) or prevention of a cancer as defined herein. Anticancer activity includes, e.g., the ability to reduce, prevent, or repair genetic damage, modulate undesired cell proliferation, modulate misregulated cell death, or modulate mechanisms of metastasis (e.g., ability to migrate).

By “treatment”, “prevention” or “amelioration” of a disease or disorder is meant delaying or preventing the onset of such a disease or disorder, reversing, alleviating, ameliorating, inhibiting, slowing down or stopping the progression, aggravation or deterioration the progression or severity of a condition associated with such a disease or disorder. In one embodiment, one or more symptoms of a disease or disorder are alleviated by at least 5%, at least 10%, at least 20%, at least 30%, at least 40%, or at least 50%.

The term “alkyl” refers to saturated non-aromatic hydrocarbon chains that may be a straight chain or branched chain, containing the indicated number of carbon atoms (these include without limitation methyl, ethyl, propyl, iso-propyl, butyl, 2-methyl-ethyl, t-butyl, allyl, or propargyl), which may be optionally inserted with N, O, or S. For example, C₁-C₆ indicates that the group may have from 1 to 6 carbon atoms in it.

The term “alkenyl” refers to an alkyl that comprises at least one double bond. Exemplary alkenyl groups include, but are not limited to, for example, ethenyl, propenyl, butenyl, 1-methyl-2-buten-1-yl and the like.

The term “alkynyl” refers to an alkyl that comprises at least one triple bond.

The term “cyclyl” or “cycloalkyl” refers to saturated and partially unsaturated cyclic hydrocarbon groups having 3 to 12 carbons, for example, 3 to 8 carbons, and, for example, 3 to 6 carbons, wherein the cycloalkyl group additionally may be optionally substituted. Exemplary cycloalkyl groups include, but are not limited to, cyclopropyl, cyclobutyl, cyclopentyl, cyclopentenyl, cyclohexyl, cyclohexenyl, cycloheptyl, cyclooctyl, and the like.

The term “heterocyclyl” refers to a nonaromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent. Examplary heterocyclyl groups include, but are not limited to piperazinyl, pyrrolidinyl, dioxanyl, morpholinyl, tetrahydrofuranyl, and the like.

The term “aryl” refers to monocyclic, bicyclic, or tricyclic aromatic ring system wherein 0, 1, 2, 3, or 4 atoms of each ring may be substituted by a substituent. Examplary aryl groups include, but are not limited to, phenyl, naphthyl, anthracenyl, azulenyl, fluorenyl, indanyl, indenyl, naphthyl, phenyl, tetrahydronaphthyl, and the like.

The term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2, 3, or 4 atoms of each ring may be substituted by a substituent. Examplary heteroaryl groups include, but are not limited to, pyridyl, furyl or furanyl, imidazolyl, benzimidazolyl, pyrimidinyl, thiophenyl or thienyl, pyridazinyl, pyrazinyl, quinolinyl, indolyl, thiazolyl, naphthyridinyl, and the like.

The term “optionally substituted” means that the specified group or moiety, such as an alkyl group, alkenyl group, and the like, is unsubstituted or is substituted with one or more (typically 1-4 substituents) independently selected from the group of substituents listed below in the definition for “substituents” or otherwise specified.

The term “substituents” refers to a group “substituted” on an alkyl, alkenyl, alkynyl, cycloalkyl, aryl, heterocyclyl, or heteroaryl group at any atom of that group. Suitable substituents include, without limitation, halogen, hydroxy, oxo, nitro, haloalkyl, alkyl, alkenyl, alkynyl, alkaryl, aryl, aralkyl, alkoxy, aryloxy, amino, acylamino, alkylcarbanoyl, arylcarbanoyl, aminoalkyl, alkoxycarbonyl, carboxy, hydroxyalkyl, alkanesulfonyl, arenesulfonyl, alkanesulfonamido, arenesulfonamido, aralkylsulfonamido, alkylcarbonyl, acyloxy, cyano or ureido. In some cases, two substituents, together with the carbons to which they are attached to can form a ring.

As used here in the term “isomer” refers to compounds having the same molecular formula but differing in structure. Isomers which differ only in configuration and/or conformation are referred to as “stereoisomers.” The term “isomer” is also used herein to refer to an enantiomer.

The term “enantiomer” is used to describe one of a pair of molecular isomers which are mirror images of each other and non-superimposable. Other terms used to designate or refer to enantiomers include “stereoisomers” (because of the different arrangement or stereochemistry around the chiral center; although all enantiomers are stereoisomers, not all stereoisomers are enantiomers) or “optical isomers” (because of the optical activity of pure enantiomers, which is the ability of different pure enantiomers to rotate planepolarized light in different directions). Enantiomers generally have identical physical properties, such as melting points and boiling points, and also have identical spectroscopic properties. Enantiomers can differ from each other with respect to their interaction with plane-polarized light and with respect to biological activity.

The designations “R and S” are used to denote the absolute configuration of the molecule about its chiral center(s). The designations may appear as a prefix or as a suffix; they may or may not be separated from the isomer by a hyphen; they may or may not be hyphenated; and they may or may not be surrounded by parentheses.

The designations or prefixes “(+) and (−)” are employed to designate the sign of rotation of plane-polarized light by the compound, with (−) meaning that the compound is levorotatory (rotates to the left). A compound prefixed with (+) is dextrorotatory (rotates to the right).

The term “racemic mixture,” “racemic compound” or “racemate” refers to a mixture of the two enantiomers of one compound. An ideal racemic mixture is one wherein there is a 50:50 mixture of both enantiomers of a compound such that the optical rotation of the (+) enantiomer cancels out the optical rotation of the (−) enantiomer.

The term “resolving” or “resolution” when used in reference to a racemic mixture refers to the separation of a racemate into its two enantiomorphic forms (i.e., (+) and (−); 65 (R) and (S) forms). The terms can also refer to enantioselective conversion of one isomer of a racemate to a product.

The term “enantiomeric excess” or “ee” refers to a reaction product wherein one enantiomer is produced in excess of the other, and is defined for a mixture of (+)- and (−)-enantiomers, with composition given as the mole or weight or volume fraction F₍₊₎ and F₍₋₎ (where the sum of F₍₊₎ and F₍₋₎=1). The enantiomeric excess is defined as *F₍₊₎−F₍₋₎* and the percent enantiomeric excess by 100×*F₍₊₎−F₍₋₎*. The “purity” of an enantiomer is described by its ee or percent ee value (% ee).

Whether expressed as a “purified enantiomer” or a “pure enantiomer” or a “resolved enantiomer” or “a compound in enantiomeric excess”, the terms are meant to indicate that the amount of one enantiomer exceeds the amount of the other. Thus, when referring to an enantiomer preparation, both (or either) of the percent of the major enantiomer (e.g. by mole or by weight or by volume) and (or) the percent enantiomeric excess of the major enantiomer may be used to determine whether the preparation represents a purified enantiomer preparation.

The term “enantiomeric purity” or “enantiomer purity” of an isomer refers to a qualitative or quantitative measure of the purified enantiomer; typically, the measurement is expressed on the basis of ee or enantiomeric excess.

The terms “substantially purified enantiomer,” “substantially resolved enantiomer” “substantially purified enantiomer preparation” are meant to indicate a preparation (e.g. derived from non optically active starting material, substrate, or intermediate) wherein one enantiomer has been enriched over the other, and more preferably, wherein the other enantiomer represents less than 20%, more preferably less than 10%, and more preferably less than 5%, and still more preferably, less than 2% of the enantiomer or enantiomer preparation.

The terms “purified enantiomer,” “resolved enantiomer” and “purified enantiomer preparation” are meant to indicate a preparation (e.g. derived from non optically active starting material, substrates or intermediates) wherein one enantiomer (for example, the R-enantiomer) is enriched over the other, and more preferably, wherein the other enantiomer (for example the S-enantiomer) represents less than 30%, preferably less than 20%, more preferably less than 10% (e.g. in this particular instance, the R-enantiomer is substantially free of the S-enantiomer), and more preferably less than 5% and still more preferably, less than 2% of the preparation. A purified enantiomer may be synthesized substantially free of the other enantiomer, or a purified enantiomer may be synthesized in a stereopreferred procedure, followed by separation steps, or a purified enantiomer may be derived from a racemic mixture.

The term “enantioselectivity,” also called the enantiomeric ratio indicated by the symbol “E,” refers to the selective capacity of an enzyme to generate from a racemic substrate one enantiomer relative to the other in a product racemic mixture; in other words, it is a measure of the ability of the enzyme to distinguish between enantiomers. A nonselective reaction has an E of 1, while resolutions with E's above 20 are generally considered useful for synthesis or resolution. The enantioselectivity resides in a difference in conversion rates between the enantiomers in question. Reaction products are obtained that are enriched in one of the enantiomers; conversely, remaining substrates are enriched in the other enantiomer. For practical purposes it is generally desirable for one of the enantiomers to be obtained in large excess. This is achieved by terminating the conversion process at a certain degree of conversion.

The term “analog” as used herein refers to a compound that results from substitution, replacement or deletion of various organic groups or hydrogen atoms from a parent compound. An analog is structurally similar to the parent compound, but can differ by even a single element of the same valence and group of the periodic table as the element it replaces.

The term “derivative” as used herein refers to a chemical substance related structurally to another, i.e., an “original” substance, which can be referred to as a “parent” compound. A “derivative” can be made from the structurally-related parent compound in one or more steps. The phrase “closely related derivative” means a derivative whose molecular weight does not exceed the weight of the parent compound by more than 50%. The general physical and chemical properties of a closely related derivative are also similar to the parent compound.

As used herein, a “prodrug” refers to compounds that can be converted via some chemical or physiological process (e.g., enzymatic processes and metabolic hydrolysis) to a therapeutic agent. Thus, the term “prodrug” also refers to a precursor of a biologically active compound that is pharmaceutically acceptable. A prodrug may be inactive when administered to a subject, i.e. an ester, but is converted in vivo to an active compound, for example, by hydrolysis to the free carboxylic acid or free hydroxyl. The prodrug compound often offers advantages of solubility, tissue compatibility or delayed release in an organism. The term “prodrug” is also meant to include any covalently bonded carriers, which release the active compound in vivo when such prodrug is administered to a subject. Prodrugs of an active compound may be prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a subject, cleaves to form a free hydroxy, free amino or free mercapto group, respectively. Examples of prodrugs include, but are not limited to, acetate, formate and benzoate derivatives of an alcohol or acetamide, formamide and benzamide derivatives of an amine functional group in the active compound and the like. See Harper, “Drug Latentiation” in Jucker, ed. Progress in Drug Research 4:221-294 (1962); Morozowich et al, “Application of Physical Organic Principles to Prodrug Design” in E. B. Roche ed. Design of Biopharmaceutical Properties through Prodrugs and Analogs, APHA Acad. Pharm. Sci. 40 (1977); Bioreversible Carriers in Drug in Drug Design, Theory and Application, E. B. Roche, ed., APHA Acad. Pharm. Sci. (1987); Design of Prodrugs, H. Bundgaard, Elsevier (1985); Wang et al. “Prodrug approaches to the improved delivery of peptide drug” in Curr. Pharm. Design. 5(4):265-287 (1999); Pauletti et al. (1997) Improvement in peptide bioavailability: Peptidomimetics and Prodrug Strategies, Adv. Drug. Delivery Rev. 27:235-256; Mizen et al. (1998) “The Use of Esters as Prodrugs for Oral Delivery of (3-Lactam antibiotics,” Pharm. Biotech. ll:345-365; Gaignault et al. (1996) “Designing Prodrugs and Bioprecursors I. Carrier Prodrugs,” Pract. Med. Chem. 671-696; Asgharnejad, “Improving Oral Drug Transport”, in Transport Processes in Pharmaceutical Systems, G. L. Amidon, P. I. Lee and E. M. Topp, Eds., Marcell Dekker, p. 185-218 (2000); Balant et al., “Prodrugs for the improvement of drug absorption via different routes of administration”, Eur. J. Drug Metab. Pharmacokinet., 15(2): 143-53 (1990); Balimane and Sinko, “Involvement of multiple transporters in the oral absorption of nucleoside analogues”, Adv. Drug Delivery Rev., 39(1-3): 183-209 (1999); Browne, “Fosphenyloin (Cerebyx)”, Clin. Neuropharmacol. 20(1): 1-12 (1997); Bundgaard, “Bioreversible derivatization of drugs—principle and applicability to improve the therapeutic effects of drugs”, Arch. Pharm. Chemi 86(1): 1-39 (1979); Bundgaard H. “Improved drug delivery by the prodrug approach”, Controlled Drug Delivery 17: 179-96 (1987); Bundgaard H. “Prodrugs as a means to improve the delivery of peptide drugs”, Arfv. Drug Delivery Rev. 8(1): 1-38 (1992); Fleisher et al. “Improved oral drug delivery: solubility limitations overcome by the use of prodrugs”, Arfv. Drug Delivery Rev. 19(2): 115-130 (1996); Fleisher et al. “Design of prodrugs for improved gastrointestinal absorption by intestinal enzyme targeting”, Methods Enzymol. 112 (Drug Enzyme Targeting, Pt. A): 360-81, (1985); Farquhar D, et al., “Biologically Reversible Phosphate-Protective Groups”, Pharm. Sci., 72(3): 324-325 (1983); Freeman S, et al., “Bioreversible Protection for the Phospho Group: Chemical Stability and Bioactivation of Di(4-acetoxy-benzyl) Methylphosphonate with Carboxyesterase,” Chem. Soc., Chem. Commun., 875-877 (1991); Friis and Bundgaard, “Prodrugs of phosphates and phosphonates: Novel lipophilic alphaacyloxyalkyl ester derivatives of phosphate- or phosphonate containing drugs masking the negative charges of these groups”, Eur. J. Pharm. Sci. 4: 49-59 (1996); Gangwar et al., “Pro-drug, molecular structure and percutaneous delivery”, Des. Biopharm. Prop. Prodrugs Analogs, [Symp.] Meeting Date 1976, 409-21. (1977); Nathwani and Wood, “Penicillins: a current review of their clinical pharmacology and therapeutic use”, Drugs 45(6): 866-94 (1993); Sinhababu and Thakker, “Prodrugs of anticancer agents”, Adv. Drug Delivery Rev. 19(2): 241-273 (1996); Stella et al., “Prodrugs. Do they have advantages in clinical practice?”, Drugs 29(5): 455-73 (1985); Tan et al. “Development and optimization of anti-HIV nucleoside analogs and prodrugs: A review of their cellular pharmacology, structure-activity relationships and pharmacokinetics”, Adv. Drug Delivery Rev. 39(1-3): 117-151 (1999); Taylor, “Improved passive oral drug delivery via prodrugs”, Adv. Drug Delivery Rev., 19(2): 131-148 (1996); Valentino and Borchardt, “Prodrug strategies to enhance the intestinal absorption of peptides”, Drug Discovery Today 2(4): 148-155 (1997); Wiebe and Knaus, “Concepts for the design of anti-HIV nucleoside prodrugs for treating cephalic HIV infection”, Adv. Drug Delivery Rev.: 39(1-3):63-80 (1999); Waller et al., “Prodrugs”, Br. J. Clin. Pharmac. 28: 497-507 (1989), content of all of which is herein incorporated by reference in its entirety.

As used herein, the term “pharmaceutically-acceptable salts” refers to the conventional nontoxic salts or quaternary ammonium salts of therapeutic agents, e.g., from non-toxic organic or inorganic acids. These salts can be prepared in situ in the administration vehicle or the dosage form manufacturing process, or by separately reacting a therapeutic agent in its free base or acid form with a suitable organic or inorganic acid or base, and isolating the salt thus formed during subsequent purification. Conventional nontoxic salts include those derived from inorganic acids such as sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like. See, for example, Berge et al., “Pharmaceutical Salts”, J. Pharm. Sci. 66:1-19 (1977), content of which is herein incorporated by reference in its entirety.

In some embodiments of the aspects described herein, representative salts include the hydrobromide, hydrochloride, sulfate, bisulfate, phosphate, nitrate, acetate, succinate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like.

To the extent not already indicated, it will be understood by those of ordinary skill in the art that any one of the various embodiments herein described and illustrated can be further modified to incorporate features shown in any of the other embodiments disclosed herein.

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples do not in any way limit the invention.

### EXAMPLES

### Example 1

### Identification of Hedgehog Pathway Antagonists

Hedgehog (Hh) signaling plays an essential role in developmental processes and adult tissue homeostasis'. An increasing body of evidence identifies the Hh pathway as a contributing factor in the growth of a variety of human cancers. The loss of normal regulatory control of the Hh pathway within a subset of Hh responsive cells leads directly to the initiation of particular solid tumors, notably basal cell carcinoma (BCC), the most prevalent cancer in the Caucasian population², and medulloblastoma (MB), the most common childhood brain cancer³. In other cancers, Hh signals from tumor cells appear to condition the local environment to favor tumor growth. This category includes a broad spectrum of high incidence cancers, particularly those in breast, lung, liver, stomach, pancreas, prostate, and gastro-intestinal tract^{4,5}. The potential of Hh targeted cancer therapy has stimulated an extensive search for Hh pathway antagonists. Typically, drug discovery screens have broadly sampled the Hh pathway looking for agents capable of silencing a Hh signal-dependent transcriptional response. Although small-molecule “hits” may occur at any point in the pathway that can ultimately translate into an altered transcriptional response, Smoothened (Smo), has emerged as the prevalent target.^{6,7} Smo is essential for all pathway activity, and activating mutations in Smo have been observed in both human BCC and MB. Smo antagonists have entered clinical trials⁸, and successful repression of tumorigenesis in patients with invasive or metastatic forms of BCC has validated the concept of Hh targeted cancer therapy⁹.

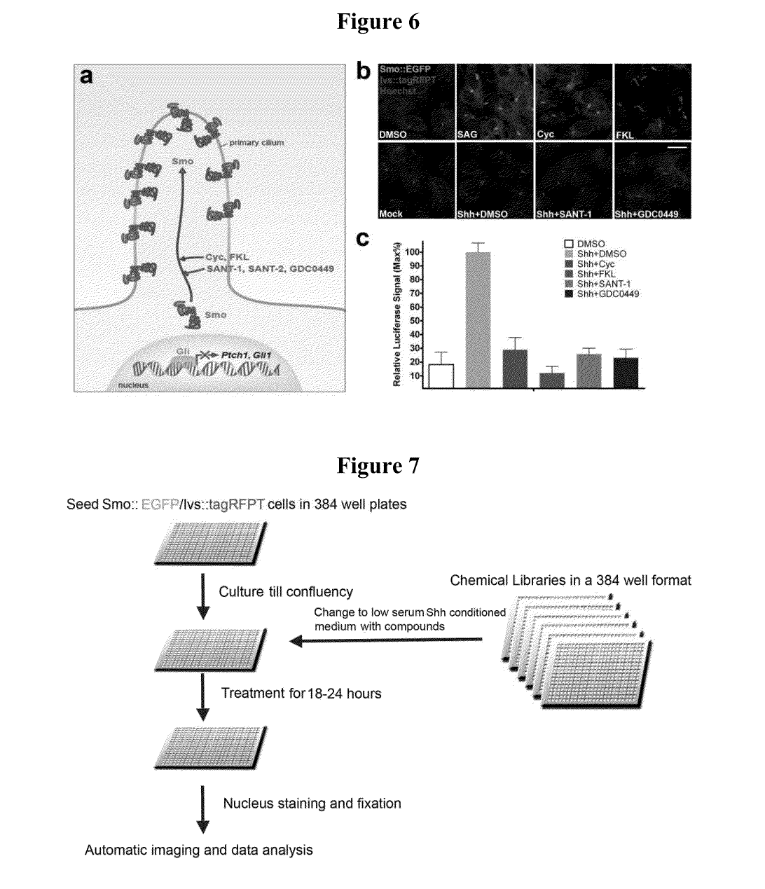

An obligatory step in the activation of Hh signaling is the accumulation of Smo in the primary cilium (PC), a tubulin-scaffolded membrane extension templated by the centriole (FIG. 6). While all small molecule Smo agonists examined so far induce Smo accumulation in the PC, various Smo antagonists affect Smo localization in distinct ways (FIG. 6)¹⁰⁻¹². SANT-1, SANT-2, and GDC0449 (also known as RG3616), a Smo antagonist in clinical trials, inhibit both Hh pathway activation and Sonic hedgehog (Shh) induced Smo accumulation within the PC¹⁰⁻¹². In contrast, Cyclopamine (cyc), a natural product from the plant Veratrum californicum, and its potent derivative KAAD-cyc, bind Smo and inhibit pathway activation, but behave as pseudo-agonists promoting Smo accumulation within the PC¹⁰⁻¹³. Further, forskolin (FKL), a putative protein kinase A (PKA) activator, inhibits Hh pathway activity and indirectly promotes Smo ciliary accumulation through PKA stimulation¹⁰. Thus, there are distinct actions and outcomes associated with different inhibitory factors grouped around Smo action (FIG. 6).

To explore regulatory activity at this critical level of pathway action, we performed a direct screen for inhibitors of Smo translocation to the PC and identified 20 classes of inhibitory compounds. We identified some compounds that can act on Smo in a similar manner to previously identified antagonists and agonists, underscoring the chemical diversity of compound interactions at what is possibly a common site. However, we also identified a compound, SMANT, which inhibits an oncogenic form of Smo refractory to inhibition by currently available Smo antagonists.

### Materials and Methods

Cell Culture:

NIH/3T3 cells were maintained in DMEM containing 10% (v/v) calf serum, penicillin, streptomycin, and L-glutamine. HEK293, L, cos 7, and suFU−/− mouse embryonic fibroblast cells were maintained in DMEM containing 10% (v/v) fetal bovine serum, penicillin, streptomycin, and L-glutamine. Smo::EGFP, SmoM2::GFP, and Ivs::tagRFPT were cloned into pBabe to generate retroviral particles for infection. Smo::EGFP/Ivs::tagRFPT and SmoM2::GFP/Ivs::tagRFPT stable cell lines were generated through viral infection of NIH/3T3¹². A ShhLightII Gli reporter cell line was obtained from the American Type Culture Collection (ATCC) and used in luciferase reporter assays to measure Hh pathway activity. The cell line contains a stably integrated Gli-responsive Firefly luciferase reporter and a constitutive Renilla luciferase expression construct³³. Subclones expressing Smo or SmoM2 in ShhLightII cells were used for chemical epistasis analyses.

Shh conditioned medium, which is collected from cos 7 cells transfected with an expression construct encoding the amino terminal 19 kDa signaling peptide of Shh, was used at 13.7(±3.0) nM. Wnt3a conditioned medium was collected from an L-cell line producing Wnt3a ligand⁴⁰. Controls utilized supernatants from cells cos 7 cells transfected with empty vector or a wild-type L-cell line.

Reagents:

Cyclopamine and forskolin were purchased from Sigma. SAG, SANT-1, GDC0449, and BODIPY-cyclopamine were purchased from Axxora Platform, Tocris Biosciences, Selleck Chemicals, and Toronto Research Chemicals, respectively. All small molecule stock solutions were prepared by dissolving in DMSO at 10 mM and stored at −20° C. Mouse recombinant ShhN purified protein (IIShhN) was purchased from R&D Systems.

Transfection was performed using Fugene6 or Fugene HD from Roche.

Imaging Assays:

Cells were cultured and treated in 384-well imaging plate precoated with poly-D-Lysine (Greiner Bio-one), fixed with 4% paraformaldehyde (Electron Microscopy Sciences), and stained with Hoechst (Invitrogen). Images were collected using Opera High Content Screening System (Perkin Elmer). Activityl)ase (IDBS Inc, Emeryvill, Calif.) and Pipeline Pilot (Accelrys, Inc. San Diego, Calif.) were used for high content screening data management and analysis. Acapella 2.0 software (Evotec Technologies/PerkinElmer) was used to perform multi-parametrical high content image quantification. All the images for comparison were scanned with identical microscopic settings and analyzed with the same input parameters.

Image Analysis of Smo Ciliary Localization:

Acapella 2.0 software (Evotec Technologies/PerkinElmer) was used to perform multi-parametric high content image quantification. Our image analysis script used Ivs::tagREPT to first determine the location of the PC and then Smo::EGFP to quantify the level of Smo present in the cilium.

The inventors used the spot-finding algorithm in the RFP channel to find Ivs-rich spots. Each non-overlapping spot was based on a maximum 8 pixel-radius (when using a 40× objective) around a central intensity peak of 5 pixels. A distance of at least 10 pixels was required between adjacent spot centers. Spot peaks had to exceed thresholds of relative intensity compared to the remaining body of the spot as well as to the entire image. The average, maximum and total RFP intensity of each spot were measured. The spots with sufficiently high absolute maxima to pass the selected threshold were classified as positive spots. To form candidate cilia, the inventors selected the brightest 15% of the pixels in each positive spot and merged those pixels into objects so that the brightest parts of adjacent spots could form single, larger cilia-shaped objects. To qualify the candidate cilia as true Ivs-positive cilia, the width of the objects created from merging the brightest pixels in each spot had to be at least 2 pixels and the length to half-width ratio had to exceed 3. (i.e. a 3-pixel long by 2-pixel wide cilium was the minimum accepted cilium size and the length had to be at least 1.5 fold of the width). The mean GFP intensity within these Ivs-positive cilia was used to estimate the ciliary level of Smo protein. The inventors subtracted the background of the mean GFP intensity in the 3-pixel wide area around each candidate cilium to avoid some false positives. Those Ivs-positive cilia that exceed the final mean GFP intensity threshold set for each experiment were deemed Smo-positive cilia.

Hoechst staining was used to determine the total number of nuclei per well.

The final output measurements of the number of Ivs-positive cilia in the well, the number of Smo-positive cilia in the well and the mean GFP intensity of the accepted cilia within the well were used to calculate if a compound qualified as an inhibitor and to estimate the quality of inhibition Inhibitors of smoothened accumulation into the cilia were initially chosen as compounds that had numbers of Ivs-positive cilia per nucleus (or per field of view) similar to the DMSO controls, but fewer Smo-positive cilia compared to the DMSO controls. Compounds that generated many fewer Ivs-positive cilia were judged as either defective in cilium assembly/trafficking or generally toxic depending on the morphology of the cells. Measurements of the geometry of the cilia as well as the total fluorescent intensities of each cilium in the Smo- and Ivs-channels were used to determine if any of the compounds were having unusual effects on cilia size, intensity or frequency of observation or combinations of all three characteristics.

The thresholds and parameters used in selecting, classifying, and quantitating spots, candidate cilia and nuclei, were applied uniformly for every well in each set of plates prepared as a single batch with the same set of cells and reagents. The inventors used diagnostic images during threshold selection to outline which objects (spots, candidate cilia, candidate nuclei etc) passed or failed each selection. At least 2 fields of positive controls (Shh+SANT-1) and negative controls (Shh+DMSO) were examined for threshold setting. Visual observation and Z-prime calculations measuring the ability of the assay to distinguish positive from negative controls were used for quality control on each batch of plates and set of thresholds. All the images for comparison were scanned with identical microscopic settings and analyzed with the same input parameters.

Hh and Wnt Activity Assays:

Hh activity assays were performed using ShhLightll cells, Smo/Lightll cells, SmoM2/Lightll cells, and suFU−/− mouse embryonic fibroblasts. In the suFU−/− cells, Hh activity was measured after co-transfection with Gli driven firefly luciferase and TK-renilla luciferase reporters⁴¹. Wnt activity was measured in 293 cells co-transfected with Top-flash and TK-renilla luciferase reporters⁴². Cells were cultured and treated in 96 well assay plates (Corning) and incubated with Duo-Glo luciferase substrates (Promega) to measure firefly and renilla luciferase activity sequentially using a Top Count NX Microplate Scintillation and Luminescence Counter (Perkin Elmer). The renilla luciferase signal was used to normalize the firefly reporter activity.

Bodipy-Cyclopamine Competition Assays:

Cos 7 cells were transfected with a plasmid co-expressing Smo and a nuclear localized form of tagRFPT (pCIT-Smo). An empty parental construct (pCIT), and a construct that co-expresses SmoM2, were used as controls to assess specificity and background noise. Three days after transfection, cells were incubated with 5 nM Bodipy-cyclopamine, with or without other compounds for 1 hour at 37□ C. Cells were washed, fixed and stained with Hoechst. Images were collected by an Opera High Content Screen System. Bodipy fluorescence was quantified specifically for transfected cells (determined by red tagRFPT+ nucleus) using a program developed by the authors with Acapella 2.0 software. All of the images were scanned with identical microscopic settings and analyzed with the same input parameters.

CGNP Proliferation Assays:

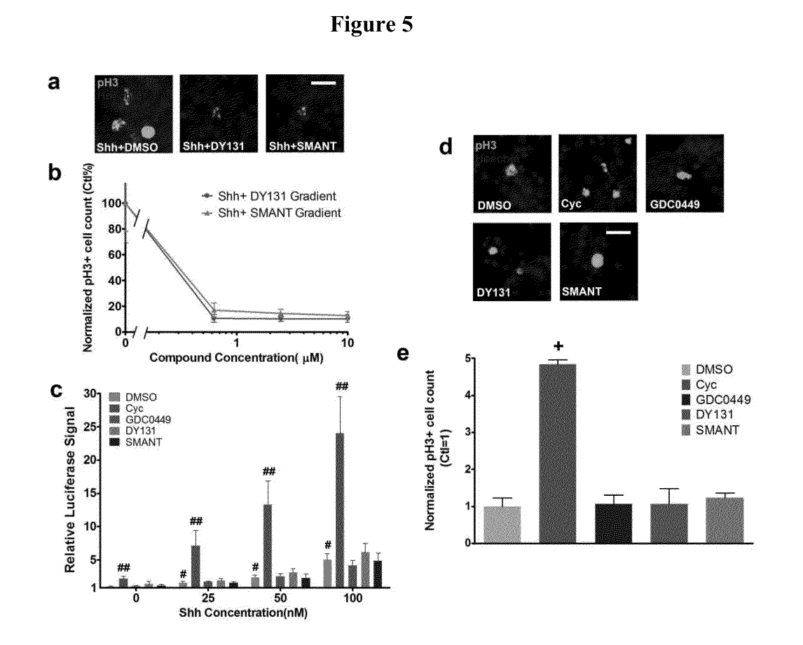

CGNP primary cells were isolated from P7 Ptch1+/− mice as previously reported⁴³ and immediately seeded in poly-D-lysine coated imaging plates (Greiner Bio-one). Compounds were applied 2 hours post seeding, for either 36 (FIG. 5a-b) or 72 hours (FIG. 5d-e). After completion of each experimental regimen, cells were fixed with 4% paraformaldehyde (Electron Microscopy Sciences), and stained with anti-pH3 antibody (Upstate; 1:100) followed by a secondary antibody (Invitrogen) and Hoechst (Invitrogen). Images were then collected using a confocal microscope. Cell proliferation, as marked by a pH3 signal, was quantified with an in-house program developed by the authors using Acapella 2.0 software. Identical microscopic settings were used in each analysis and identical input parameters were implemented for each experiment.

### Results and Discussion

Screening for Antagonists of Smo Translocation to the Primary Cilium.

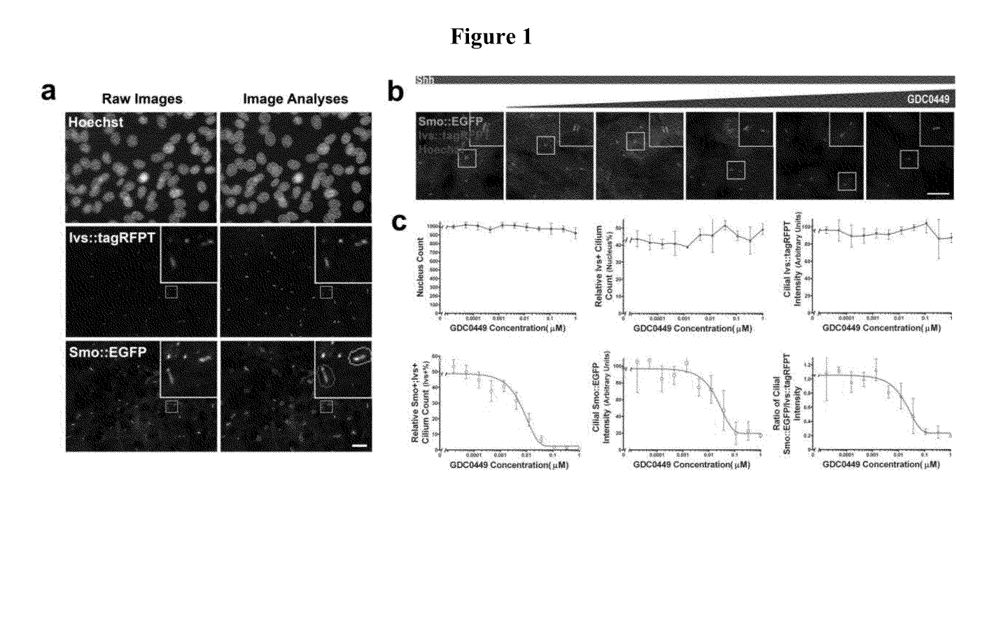

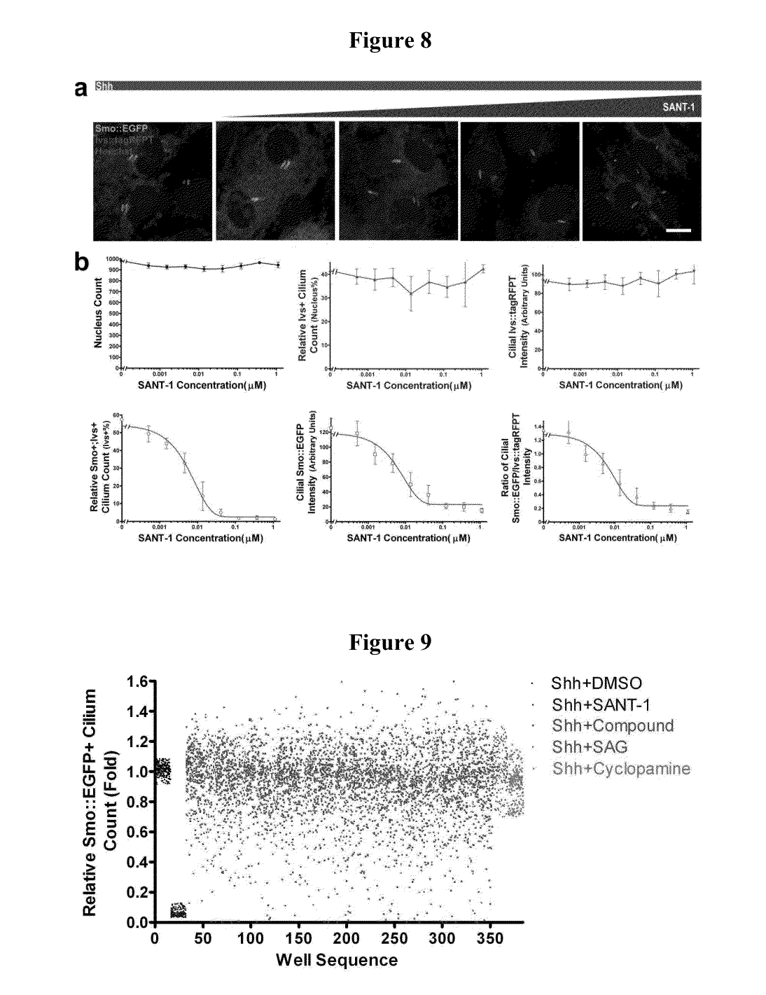

In work to be published elsewhere, we have established a high content screen for modulators of Smo translocation focusing on small molecules stimulating Smo translocation to the PC (Wang Y. et al., under revision). We then modified the system to identify inhibitors of Smo ciliary translocation. In brief, we developed a cell line producing human Smo::EGFP and Ivs::tagRFPT fusion proteins. Ivs::tagRFPT highlights the PC, and GFP enables the cellular trafficking of Smo to be visualized. Test compounds were added in low serum medium for 18-24 hours in the presence of Shh, cells were then fixed and stained with Hoechst (FIG. 7). Quantitative multi-parametric image analyses were performed with custom algorithms. The most critical parameters measured are indicated in FIG. 1a: Cell number was measured by counting Hoechst-labeled nuclei whereas the PC was precisely segmented as an Ivs::tagRFPT positive structure; the specific PC localization of Smo::EGFP was discerned by applying a defined threshold for the length-width ratio of Ivs::tagRFPT positive structures (inset in Ivs::tagRFPT images) and then quantifying hSmo::EGFP intensity within the Ivs::tagRFPT positive PC. Key measurements from these analyses are shown for GDC0449 (FIGS. 1b and c) and SANT-1 (FIG. 8). As expected, each specifically inhibited Smo::EGFP accumulation in the PC without causing significant structural changes to the PC itself or measurable cytotoxicity^{18,20}.

We used this high content assay to screen a collection of approximately 5,600 small molecules for compounds that block Smo accumulation in the presence of Shh. The small molecule library includes FDA approved drugs, drug candidates in preclinical or clinical development and a group of compounds with annotated biological activity. Representative examples of assay plates are shown (FIG. 9). Z-prime scores²¹ were consistently >0.6, confirming the robustness of the screen.

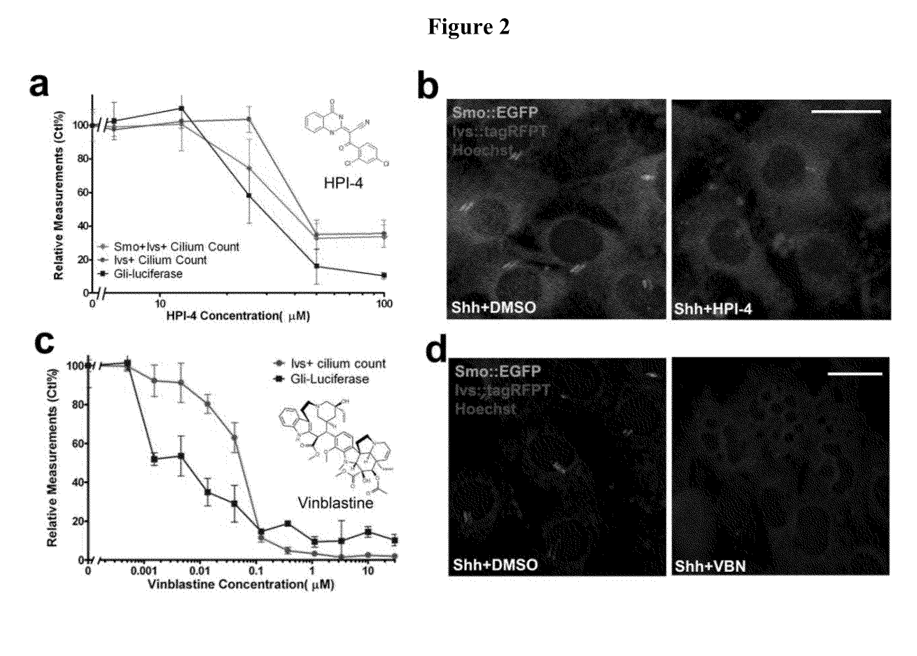

We first eliminated small molecules with “off-target” effects, e.g. inhibitory effects on ciliary assembly/trafficking or general cytotoxicity. For example, HPI-4, a molecule that leads to truncation or loss of the PC²², and vinblastine, a drug known to disrupt the assembly of microtubules²³, both appear as Hh pathway antagonists in a Gli-luciferase reporter assay (FIG. 2 a and c). However, the decrease in the Ivs::tagRFPT ciliary signal in the Smo high-content assay indicates a non-specific mechanism that alters PC structure (FIG. 2).

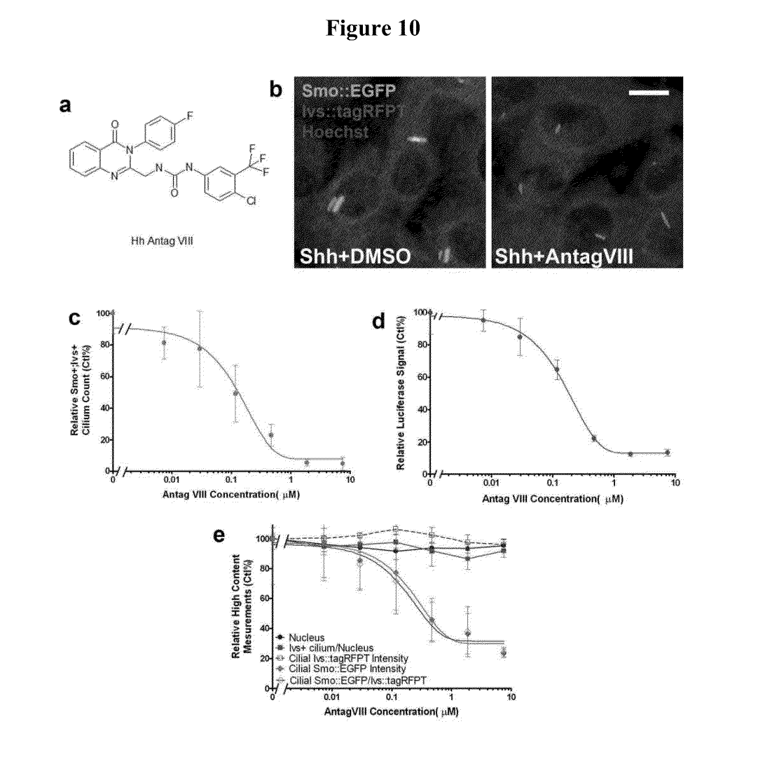

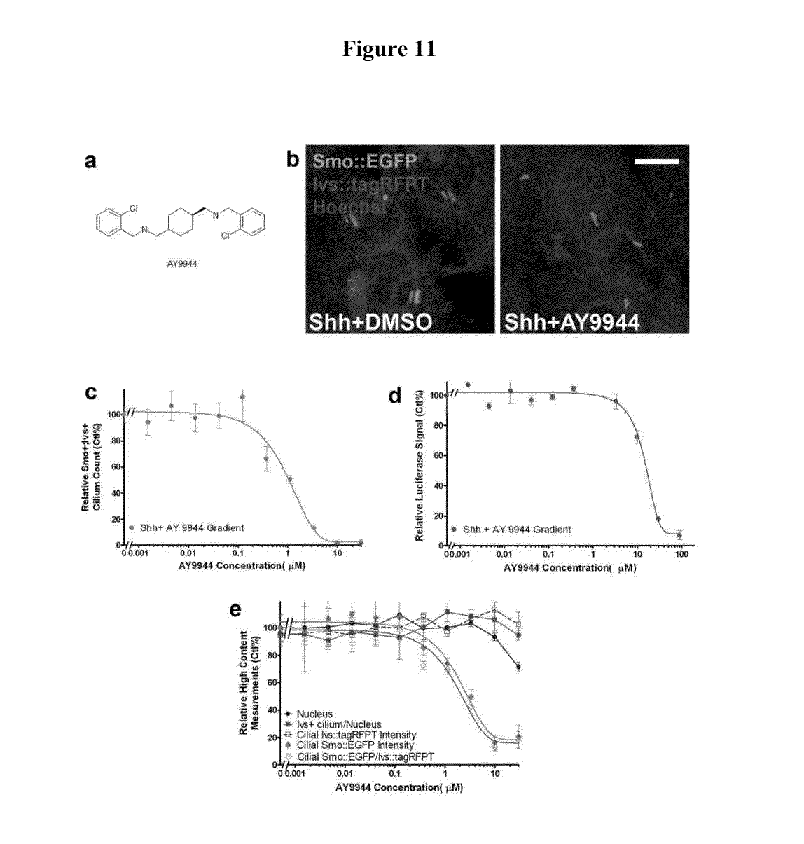

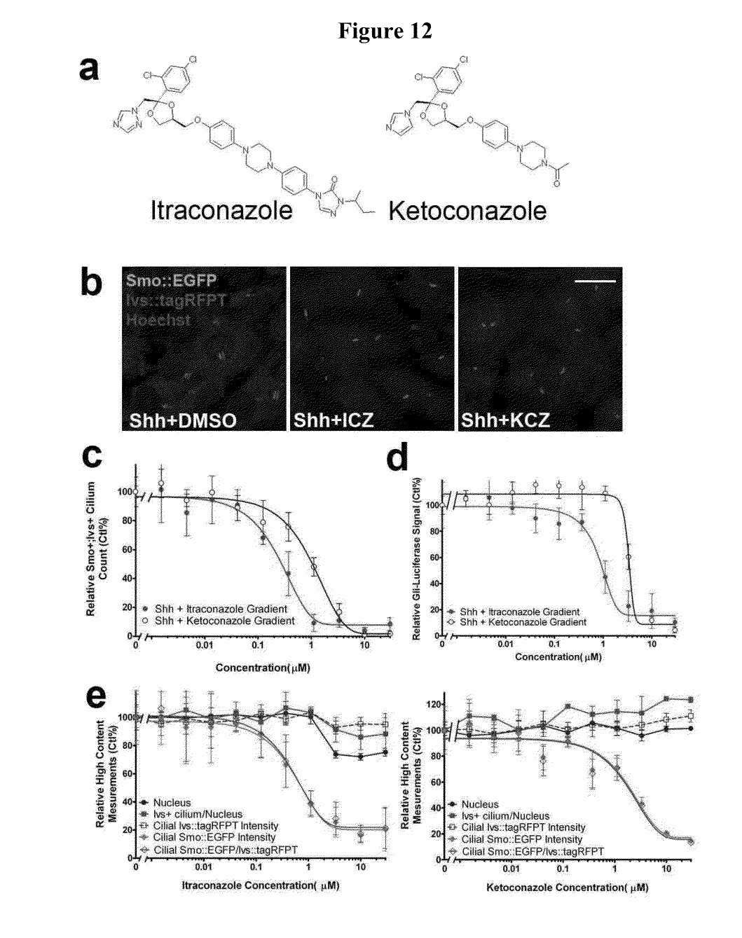

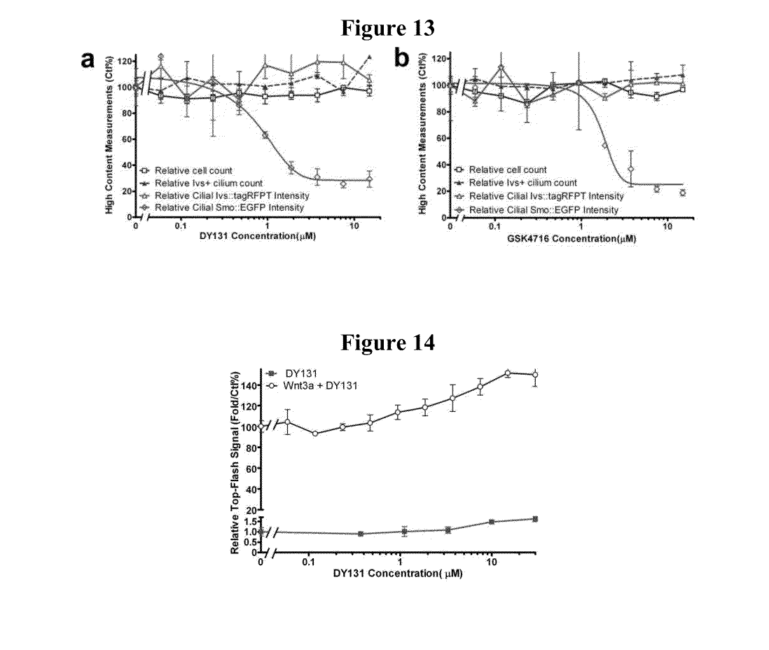

We identified 26 validated hits that could be divided into 20 classes. These hits include known Hh pathway inhibitors such as AntagVIII, a potent phenyl quinazolinone urea derivative (FIG. 10)²⁴. Moreover, identification of AY9944, an inhibitor of cholesterol biosynthesis and esterification²⁵, adds additional support to the proposed intersection between cholesterol metabolism and the Hh pathway²⁶. Hh ligands are covalently modified by cholesterol²⁷ and Hh trafficking has been linked to cholesterol transport processes^{28,9} but in vitro studies suggest the response of the target cell is actually suppressed when cholesterol biosynthesis is blocked²⁶. Our data suggest a potential link with Smo accumulation within the PC (FIG. 11). Further, in line with a recent report³⁰, our screen identified itraconazole and ketoconazole, two anti-fungal drugs in current clinical use, as Smo inhibitors in the ciliary-based assay (FIG. 12). In all cases examined, compounds that blocked Smo translocation to the PC inhibited Gli transcription activity, often with comparable IC50's (FIGS. 10-12).

DY131 Inhibits Smo Signaling Through a Conventional Mechanism.

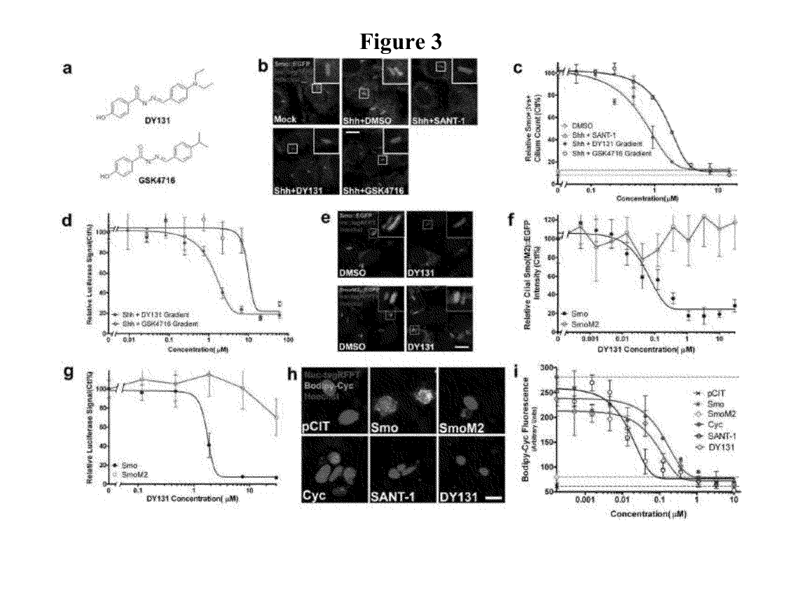

Of the novel compounds, we first selected DY131, a potent hit, for subsequent analysis. DY131, and its analog GSK4716, inhibited Shh induced accumulation of Smo::EGFP with IC50's of 0.8 μM and 2 μM respectively (FIGS. 3a-c and 13, and Table 1). Both DY131 and GSK4716 inhibit Shh induced activation of a Gli-reporter with somewhat higher IC50's (2 μM and 10 μM respectively) (FIG. 3d). The absence of an inhibitory activity in a Wnt pathway reporter assay argues for a specific action of DY131 in suppressing Shh action (FIG. 14).

TABLE 1
IC₅₀'s of Newly Identified Smo Antagonists in Various Cell-Based Assays^{a}

| cell line | stimulus | measurement | DY131 | SMANT |
|---|---|---|---|---|
| 3T3/Smo::GFP/Ivs::tagRFPT | Shh | inhibition of Shh-induced | 0.8 | 1.1 |
| | | Smo::GFP ciliary accumulaton | | |
| 3T3/Shh-LightII | Shh | inhibition of Shh-induced | 2 | 2 |
| | | expression of Gli-luciferase reporter | | |
| 3T3/Smo::GFP/Ivs::tagRFPT | SAG | inhibition of SAG-induced | 2 (100 | >60 (100 |
| | | Smo::GFP ciliary accumulation | nM SAG) | nM SAG) |
| 3T3/Shh-LightII | SAG | inhibition of SAG-induced expression | 2 (100 | 3 (200 nM SAG); |
| | | of Gli-luciferase reporter | nM SAG) | 4 (1 μM SAG) |
| 3T3/Smo::GFP Overexpression/Ivs::tagRFPT | none | inhibiton of ciliary accumulation | 0.08 | 3 |
| | | of Smo::GFP upon overexpression | | |
| 3T3/Smo::GFP Overexpression/Shh-LightII | none | inhibition of expression of Gli-luciferase | >30 | 3 |
| | | reporter induced by Smo::GFP overexpression | | |
| 3T3/SmoM2::GFP Overexpression/Ivs::tagRFPT | none | inhibition of ciliary localization of SmoM2::GFP | >30 | >60 |
| 3T3/SmoM2::GFP Overexpression/Shh-LightII | none | inhibition of expression of Gli-luciferase | >30 | 1.2 |
| | | reporter induced by SmoM2::GFP overexpression | | |
| Cos7/Smo expression | none | competition of BODIFY-cyclopamine-Smo binding | 0.1 | >30 |
| Ptch ± CGNP | Shh | Shh induced cell proliferation marked by pH 3 | <0.625 | <0.625 |

^{a}Please note that IC₅₀'s in this paper were obtained through nonlinear regression based on the following equation: Y = bottom + (top − bottom)/(1 + 10^{X-log IC}^{₅₀}), where the top and bottom are the Y values of plateaus of no inhibition and saturated inhibition separately.

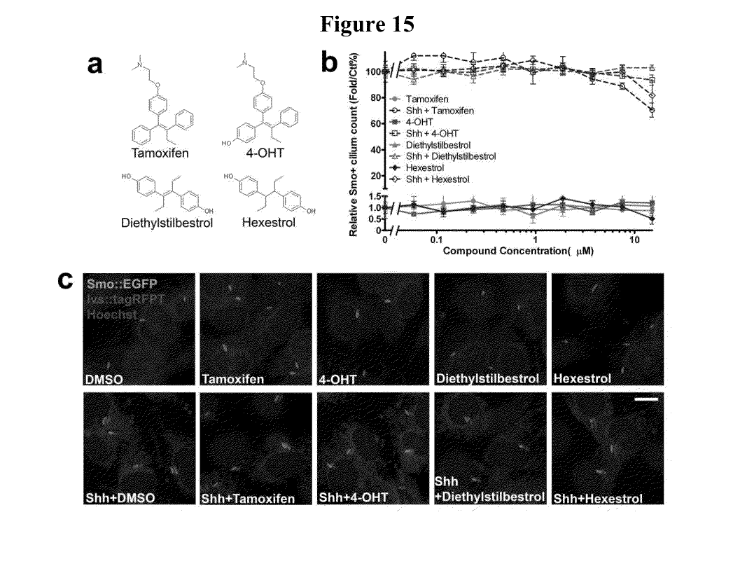

DY131 and GSK4716 were previously identified as agonists of the estrogen related receptors (ERR)^{31,32}. However, other ERR/ER ligands, including tamoxifen citrate, 4-hydroxytamoxifen (4-OHT), diethylstilbestrol, and hexestrol, did not alter the accumulation of Smo on the PC in either the presence or absence of Shh (FIG. 15), arguing against an ERR-based mode of action for DY131 and GSK4716.

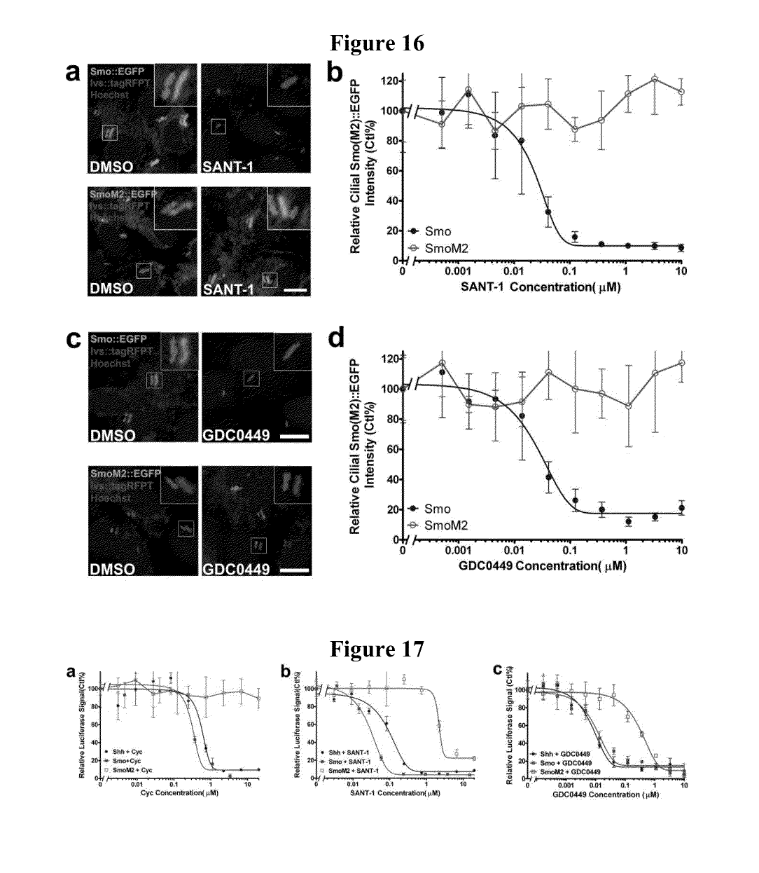

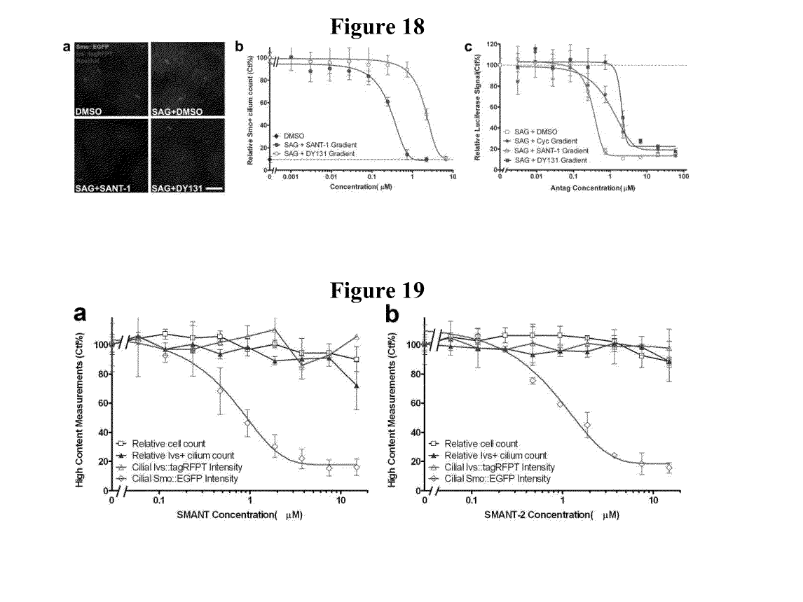

To investigate at what level DY131 functions in the Hh pathway, we compared the drug's dose-dependent performance in inhibiting the activities of wild-type Smo and SmoM2 (also named SMOA1), a constitutively active form of Smo with a tryptophan to leucine mutation in the 7^{th} transmembrane domain². This mutation renders Smo markedly less sensitive (IC50's for SmoM2 are at least an order of magnitude higher than IC50's for wildtype Smo.) to Cyc, SANT-1 and GDC0449-mediated inhibition^{18,20,33} (FIGS. 16 and 17). When over-expressed, both wild-type Smo and SmoM2 constitutively localize to the primary cilium^{6,34}. In contrast to its potent inhibition of the ciliary accumulation of wild-type Smo following exposure to Hh ligand (FIG. 3c), or over-expression of wild-type Smo (FIG. 3 e and f), DY131 failed to inhibit ciliary localization of SmoM2, or SmoM2 driven activation of transcriptional reporters of pathway activity, at doses up to 30 μM (FIG. 3 e-g). However, DY131 suppressed SAG (100 nM) induced accumulation of Smo::EGFP in the primary cilium and Gli transcription activity with an IC50 of approximately 2 μM (FIG. 18).

Interestingly, SANT-1 and GDC0449, at a dose high enough to block SmoM2 activity, did not alter SmoM2 ciliary accumulation, suggesting that, as with wild-type Smo, activity of this mutant can be abolished without blocking its localization to the PC (FIG. 16).

To determine if DY131 binds directly to Smo, we used a competition assay with bodipy-Cyc, a fluorescent analog of Cyc³⁵. Bodipy-Cyc specifically labels cells over-expressing Smo, co-expressing a red, nuclear fluorescent protein (Nuc-tagRFPT) marker, whereas SmoM2-expressing cells do not bind bodipy-Cyc, confirming the specificity of this assay (FIG. 3 h and i). DY131, like Cyc and SANT-1, acts as an effective competitor of bodipy-Cyc labeling of cells overexpressing Smo, consistent with either direct binding to Smo at the same site as bodipy-Cyc, or at another site on Smo resulting in allosteric modification and loss of bodipy-Cyc binding (FIG. 3 h and i).

SMANT Inhibits Smo Signaling with a Novel Mechanism.

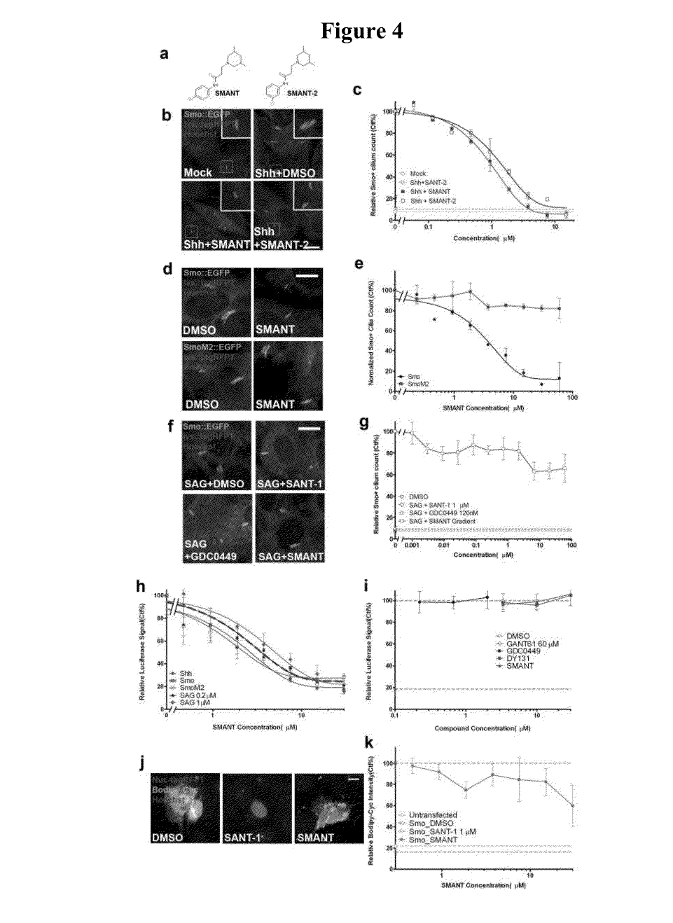

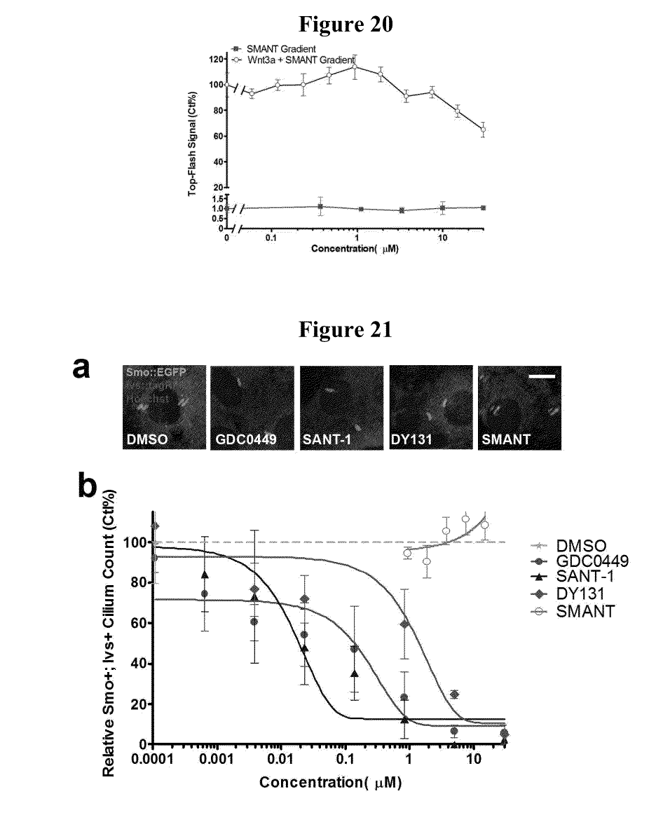

Our data indicates that DY131 and its analogs inhibit Hh signaling through a similar mechanism to inhibitors such as Cyc, SANT-1, and GDC0449. However, our focused effort in characterizing most potent hits from the screen also identified small molecules displaying novel behaviors. We named one compound, Smo Mutant ANTagonist (SMANT) as it exhibited an equivalent activity in inhibiting SmoM2 and wild-type Smo (FIGS. 4a and h). SMANT and its analog SMANT-2 inhibited Shh induced ciliary accumulation of Smo::EGFP with IC50's of 1.1 μM and 1.6 μM, respectively (FIGS. 4b and c). Neither resulted in altered Ivs::tagRFPT localization at the PC or profound modulation of Wnt pathway activity (FIGS. 4b, 19, and 20), consistent with a Hh pathway specific mode of action.

As with DY131, SANT-1, and GDC0449 (FIGS. 3e, 3f and 16), SMANT failed to block SmoM2::EGFP localization to the PC while potently inhibiting wild-type Smo accumulation (FIGS. 4d and 4e). In contrast to some of the other Smo antagonists, SMANT failed to block Smo ciliary localization induced by SAG or Cyc (FIGS. 4f, 4g, 8a, 8b, and 21). However, in contrast to other pathway inhibitors, SMANT was similarly effective at inhibiting Smo and SmoM2 activity, and blocked the stimulatory action of SAG in the Gli-luciferase assay (FIGS. 3d, 3g, 4h; 17 and 18c). SMANT, like DY131 and GDC0449, and distinct from GANT61³⁶, a known Gli inhibitor, does not alter Hh pathway activation induced by loss of suFU, a Gli regulatory factor (FIG. 4i), suggesting that SMANT functions at the Smo level. However, in contrast with strong competition between DY131 and Cyc for binding Smo (FIGS. 3h and i), SMANT was a poor competitor (FIGS. 4j and k), consistent with a unique inhibitory action on Smo activity.

DY131 and SMANT Effectively Inhibit Hh Signaling without the Risk of Rebound Hyperactivity.

To further explore the potential utility of compounds found in our assay for developing anti-cancer agents for Hh pathway targeted therapies, we tested DY131 and SMANT on cultured cerebellar granule neuron precursors (CGNPs) isolated from Ptch1+/− neonates. Constitutive activation of Hh signaling in these cells is associated with medulloblastoma³⁷. Consistent with their potency in inhibiting Hh activity in NIH/3T3 cells, DY131 and SMANT dramatically decreased phosphorylated histone H3 (pH3) marked proliferation of CGNPs induced by Shh (FIG. 5 a-b).

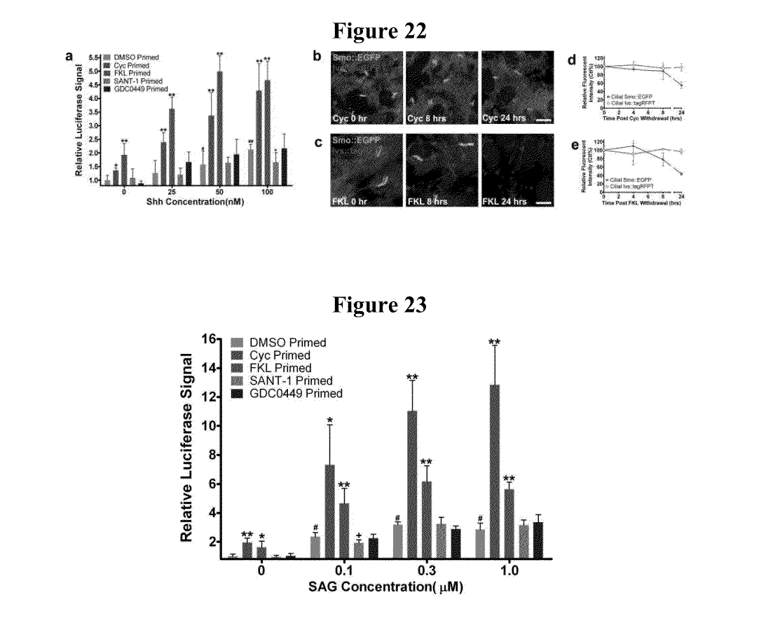

Finally, we primed cells with GDC0449, Cyc, FKL or SANT-1 at doses sufficient to decrease both Smo ciliary localization and Gli mediated transcription activity for 24 hours (FIG. 6). Following the removal of drug containing medium, and extensive washing, cells were stimulated with either Hh ligand, or the direct-binding Smo agonist SAG^{17,18}. As predicted, we observed an elevated signaling response specifically in Cyc and FKL treated cells (FIGS. 22a and 23). The hypersensitivity to Hh pathway activation correlated with high levels of Smo that remained within the PC following removal of the antagonizing compound (FIGS. 22b-22e). Next we tested the consequences of this effect for newly identified DY131 and SMANT using NIH3T3 cells (FIG. 5c) and CGNPs (FIG. 5 d-e). In contrast to Cyc, we observed no Shh driven hyperactivation of Hh pathway activity on removal of DY131 or SMANT in either the NIH3T3 Gli-luciferase assay or the CGNP proliferation assay.

Autocrine and paracrine Hedgehog (Hh) signaling promote tumorigenesis. The accumulation of Smoothened (Smo) to the primary cilium (PC) is a key event in Hh signal transduction and many pharmacological inhibitors identified to date target Smo's actions. While some antagonists (SANT-1, SANT-2, and GDC0449) block Smo translocation to the PC, cyclopamine (Cyc) stimulates Smo accumulation within the PC, triggering a hypersensitive state to renewed Hh signaling when inhibitory compounds fall below an effective concentration. To identify novel inhibitory compounds acting on the critical mechanistic transition of Smo accumulation, we established a high content screen to directly analyze Smo ciliary translocation. Screening thousands of compounds from annotated libraries containing approved drugs and other agents, we identified several new classes of compounds that block Sonic hedgehog-driven Smo localization within the PC. Selective analysis was conducted on two classes of Smo antagonist. One of these, DY131, previously described as an agonist of the estrogen related receptor, inhibits Smo signaling through a binding site shared by Cyc, SANT-1, and GDC0449. The other, SMANT, inhibited Smo activity induced by Hh or Smo overexpression and the Smo binding agonist SAG. SMANT also inhibited an oncogenic mutant form of Smo, SmoM2, previously identified as refractory to other inhibitory molecules. Inhibition occurred without inducing a secondary hypersensitivity to pathway activation. Our observations identify important differences among Hh antagonists and point to new opportunities for developing antagonists that may work effectively against mutant forms of Smo resistant to current therapeutic approaches.

Based on evidence presented here, compounds that inhibit both pathway activity and Smo accumulation in the primary cilium have characteristics that makes them reasonably preferred to antagonists that themselves promote ciliary accumulation of Smo. Therefore, the type of high content screen that the inventors have established, which directly quantifies the Smo-PC interactions required for Hh pathway activity, is useful both for discovering new classes of antagonists, as well as for studying existing ones. The current screen, of over 5,000 compounds, selectively identified a substantial number of small molecules with efficacy in this assay, and more conventional Hh pathway assays. While careful analysis of DY131 suggests a direct interaction with Smo, SMANT shows a unique profile inhibiting an oncogenic form of Smo carrying the M2 mutation with similar efficacy to its wild-type counterpart. The differing properties of SMANT when compared with a variety of other Smo modulators (SAG, Cyc, GDC0449, and SANT-1) are consistent with a SMANT inhibitory action at a different site on Smo to that bound by these other compounds, or an indirect modulation of Smo activity. Smo can be inactivated in the PC by SMANT when harboring the M2 mutation, or after SAG driven translocation to the PC, suggesting that SMANT may inactivate both the oncogenic form and an SAG-bound form of Smo, and more importantly, the ciliary localization of Smo and its activation may be mechanistically divergent. It is possible that post-translational modifications, conformation changes, or interacting partners that regulate ciliary entry or accumulation of Smo differ from those governing activity in the primary cilium. Frequently, compounds show a higher potency in the inhibition of Smo localization to the primary cilium than that in Gli-luciferase assays (FIGS. 3C, 3D, 4C, 4H, and 10-12). This could reflect pathway activity while Smo is out of the PC, or the different time frames involved in the two assay systems.

The work described herein highlight new opportunities for therapeutic development that can potentiate existing approaches and over new strategies towards treatment of resistant forms of Smo emerging from somatic mutation. The screening platform provides a robust assay system. The Smo ciliary screen broadly interrogates a key aspect of HH pathway regulation and biology, and potentially identifies small molecule regulators that may not score in a conventional transcriptional end-point assay. These compounds may nevertheless provide a reasonable grounding for subsequent drug development. Further, the screen enables a stratification of small molecule function in the HH pathway and a platform that can be extended to potentially explore ciliopathies, an increasingly important area of medical significance³⁹.

### REFERENCES

1. McMahon, A. P., Ingham, P. W. & Tabin, C. J. Developmental roles and clinical significance of hedgehog signaling. Curr Top Dev Biol 53, 1-114 (2003).
2. Xie, J. et al. Activating Smoothened mutations in sporadic basal-cell carcinoma. Nature 391, 90-2 (1998).
3. Romer, J. T. et al. Suppression of the Shh pathway using a small molecule inhibitor eliminates medulloblastoma in Ptc1(+/−)p 53(−/−) mice. Cancer Cell 6, 229-40 (2004).
4. Yauch, R. L. et al. A paracrine requirement for hedgehog signalling in cancer. Nature 455, 406-10 (2008).
5. Rubin, L. L. & de Sauvage, F. J. Targeting the Hedgehog pathway in cancer. Nature Reviews Drug discovery 5, 1026-33 (2006).
6. Corbit, K. C. et al. Vertebrate Smoothened functions at the primary cilium. Nature 437, 1018-21 (2005).
7. Rohatgi, R., Milenkovic, L. & Scott, M. P. Patchedl regulates hedgehog signaling at the primary cilium. Science 317, 372-6 (2007).
8. Scales, S. & de Sauvage, F. Mechanisms of Hedgehog pathway activation in cancer and implications for therapy. Trends Pharmacol Sci (2009).
9. Von Hoff, D. et al. Inhibition of the Hedgehog Pathway in Advanced Basal-Cell Carcinoma. N Engl J Med (2009).
10. Wilson, C. W., Chen, M.-H. & Chuang, P.-T. Smoothened adopts multiple active and inactive conformations capable of trafficking to the primary cilium. PLoS ONE 4, e5182 (2009).
11. Rohatgi, R., Milenkovic, L., Corcoran, R. & Scott, M. Hedgehog signal transduction by Smoothened: Pharmacologic evidence for a 2-step activation process. Proc Natl Acad Sci USA (2009).
12. Wang, Y., Zhou, Z., Walsh, C. & McMahon, A. Selective translocation of intracellular Smoothened to the primary cilium in response to Hedgehog pathway modulation. Proc Natl Acad Sci USA (2009).
13. Dijkgraaf, G. J. et al. Small molecule inhibition of GDC-0449 refractory smoothened mutants and downstream mechanisms of drug resistance. Cancer Research 71, 435-44 (2011).
14. Kim, J., Kato, M. & Beachy, P. Gli2 trafficking links Hedgehog-dependent activation of Smoothened in the primary cilium to transcriptional activation in the nucleus. Proc Natl Acad Sci USA (2009).
15. Han, Y. et al. Dual and opposing roles of primary cilia in medulloblastoma development. Nat Med (2009).
16. Wong, S. et al. Primary cilia can both mediate and suppress Hedgehog pathway-dependent tumorigenesis. Nat Med (2009).
17. Frank-Kamenetsky, M. et al. Small-molecule modulators of Hedgehog signaling: identification and characterization of Smoothened agonists and antagonists. J Biol 1, 10 (2002).
18. Chen, J. K., Taipale, J., Young, K. E., Maiti, T. & Beachy, P. A. Small molecule modulation of Smoothened activity. Proc Natl Acad Sci USA 99, 14071-6 (2002).
19. Watanabe, D. et al. The left-right determinant Inversin is a component of node monocilia and other 9+0 cilia. Development 130, 1725-34 (2003).
20. Yauch, R. et al. Smoothened Mutation Confers Resistance to a Hedgehog Pathway Inhibitor in Medulloblastoma. Science (2009).
21. Zhang, J. H., Chung, T. D. Y. & Oldenburg, K. R. A simple statistical parameter for use in evaluation and validation of high throughput screening assays. Journal of Biomolecular Screening 4, 67-73 (1999).
22. Hyman, J. et al. Small-molecule inhibitors reveal multiple strategies for Hedgehog pathway blockade. Proc Natl Acad Sci USA (2009).
23. Jordan, M. A. & Wilson, L. Microtubules as a target for anticancer drugs. Nature Reviews Cancer 4, 253-265 (2004).
24. Brunton, S. A. et al. Potent inhibitors of the hedgehog signaling pathway. Journal of Medicinal Chemistry 51, 1108-1110 (2008).
25. Incardona, J. P. & Eaton, S. Cholesterol in signal transduction. Current Opinion in Cell Biology 12, 193-203 (2000).
26. Cooper, M. K. et al. A defective response to Hedgehog signaling in disorders of cholesterol biosynthesis (vol 33, pg 508, 2003). Nature Genetics 34, 113-113 (2003).
27. Porter, J. A., Young, K. E. & Beachy, P. A. Cholesterol modification of hedgehog signaling proteins in animal development. Science 274, 255-259 (1996).
28. Lewis, P. M. et al. Cholesterol modification of sonic hedgehog is required for long-range signaling activity and effective modulation of signaling by Ptcl. Cell 105, 599-612 (2001).
29. Li, Y. N., Zhang, H. M., Litingtung, Y. & Chiang, C. Cholesterol modification restricts the spread of Shh gradient in the limb bud. Proceedings of the National Academy of Sciences of the United States of America 103, 6548-6553 (2006).
30. Kim, J. et al. Itraconazole, a commonly used antifungal that inhibits Hedgehog pathway activity and cancer growth. Cancer Cell 17, 388-99 (2010).
31. Yu, D.D. & Forman, B. M. Identification of an agonist ligand for estrogen-related receptors ERRbeta/gamma. Bioorg Med Chem Lett 15, 1311-3 (2005).
32. Zuercher, W. J. et al. Identification and structure-activity relationship of phenolic acyl hydrazones as selective agonists for the estrogen-related orphan nuclear receptors ERRbeta and ERRgamma. J Med Chem 48, 3107-9 (2005).
33. Taipale, J. et al. Effects of oncogenic mutations in Smoothened and Patched can be reversed by cyclopamine. Nature 406, 1005-9 (2000).
34. Han, Y.-G. et al. Hedgehog signaling and primary cilia are required for the formation of adult neural stem cells. Nat Neurosci 11, 277-84 (2008).
35. Chen, J. K., Taipale, J., Cooper, M. K. & Beachy, P A Inhibition of Hedgehog signaling by direct binding of cyclopamine to Smoothened. Genes Dev 16, 2743-8 (2002).
36. Lauth, M., Bergström, A., Shimokawa, T. & Toftgård, R Inhibition of GLI-mediated transcription and tumor cell growth by small-molecule antagonists. Proc Natl Acad Sci USA 104, 8455-60 (2007).
37. Schuller, U. et al. Acquisition of granule neuron precursor identity is a critical determinant of progenitor cell competence to form Shh-induced medulloblastoma. Cancer Cell 14, 123-134 (2008).
38. Corcoran, R. B. & Scott, M. P. Oxysterols stimulate Sonic hedgehog signal transduction and proliferation of medulloblastoma cells. Proc Natl Acad Sci USA 103, 8408-13 (2006).
39. Nigg, E. A. & Raff, J. W. Centrioles, centrosomes, and cilia in health and disease. Cell 139, 663-78 (2009).
40. Willert, K. et al. Wnt proteins are lipid-modified and can act as stem cell growth factors. Nature 423, 448-52 (2003).
41. Nybakken, K., Vokes, S. A., Lin, T. Y., McMahon, A. P. & Perrimon, N. A genome-wide RNA interference screen in Drosophila melanogaster cells for new components of the Hh signaling pathway. Nature Genetics 37, 1323-1332 (2005).
42. Corbit, K. C. et al. Kif3a constrains beta-catenin-dependent Wnt signalling through dual ciliary and non-ciliary mechanisms. Nat Cell Biol 10, 70-6 (2008).
43. Chan, J. A. et al. Proteoglycan interactions with Sonic Hedgehog specify mitogenic responses. Nature Neuroscience 12, 409-417 (2009).

### Example 2

### Structure Activity Relationship Analyses of Inhibitory Activity of Smo Cilial Accumulation by DY131 and Analogs

A series of analogs were acquired to further confirm the DY131/GSK 4716 series, as well as develop a preliminary structure activity relationship. An abundant number of compounds with hydrazide scaffold, similar to DY131/GSK4716, exist in the commercial domain. We strictly designed the study by investigating the activity effects of making minor chemical modifications to the A and B ring of DY131. For the following assays, Smo::EGFP/Ivs::tagRFPT cells were primed with compounds for 3 hours prior to Shh addition in order to increase assay sensitivity. We first fixed one ring side, while varying the substituent's on the other ring side. FIG. 24 shows our preliminary results from Smo antagonist assays.

Based on the resulrs shown in FIG. 24, following conclusions can be drawn:
(1) Removal of substituents on Ring A and Ring B (RL-0070822) inactivated the compound.
(2) Removal of the R3 group, keeping R1=hydroxyl (RL-0070462), dramatically impaired the activity, whereas removing the R1=hydroxyl on compound RL-0070463 moderately decreased the activity.
(3) A more bulky or lipophilic substituent (e.g. H→F→NO2→-N(CH3)2) in the R3 position generally increased the activity.
(4) Chemical modifications or variation of substituent location on ring B can be tolerated (e.g. RL-0070464-methylation of the hydroxyl on DY131).

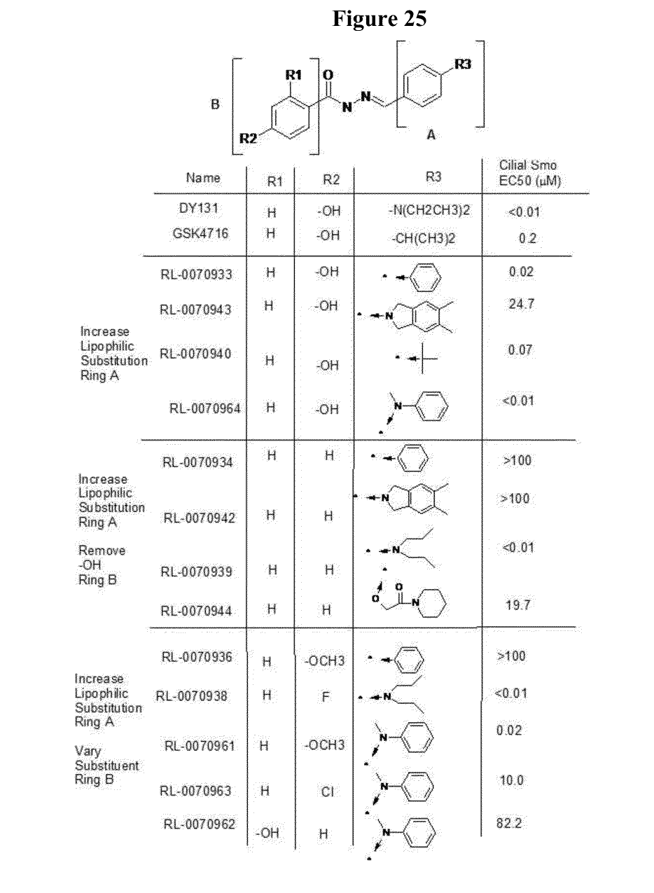

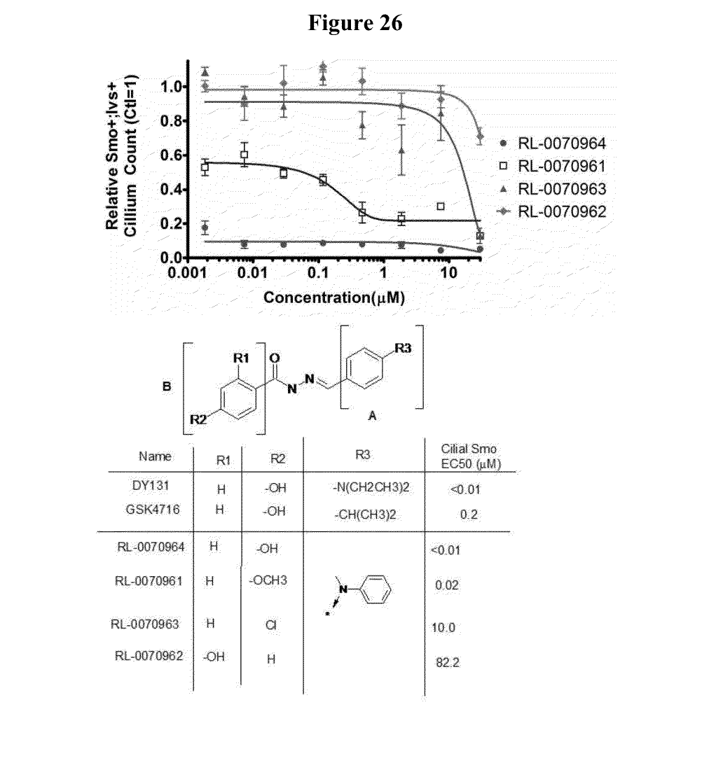

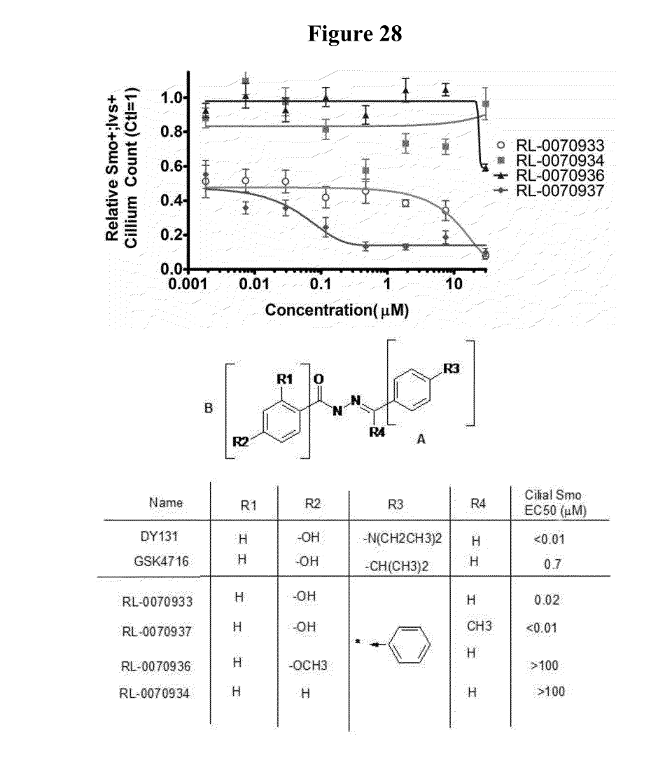

In the second round studies, we expanded upon conclusions (2), (3) and (4) from the above results. We identified several compounds where there was a more bulky substituent on ring A that still retained Smo inhibitory activity (FIG. 25; e.g. RL-0070933 and RL-0070964). Notably, a compound close to RL-0070938, in which R2=-N(CH2CH3)₂ and R3=F, was reported to be inactive in ERRγ activity¹, suggesting divergence of structural features associated with different biological activities. Compounds with the same R3 were grouped to further investigate SAR hypothesis (2) and (4) (FIGS. 26-28). Removing the R1=hydroxyl impairs the activity when R3=phenyl (FIG. 28) while retaining the activity when R3=diethyl-amine (FIG. 4). Moreover, whether it is tolerated to have chemical modifications or variation of substituent location on ring B also depends on the R3 group (FIG. 26-28).

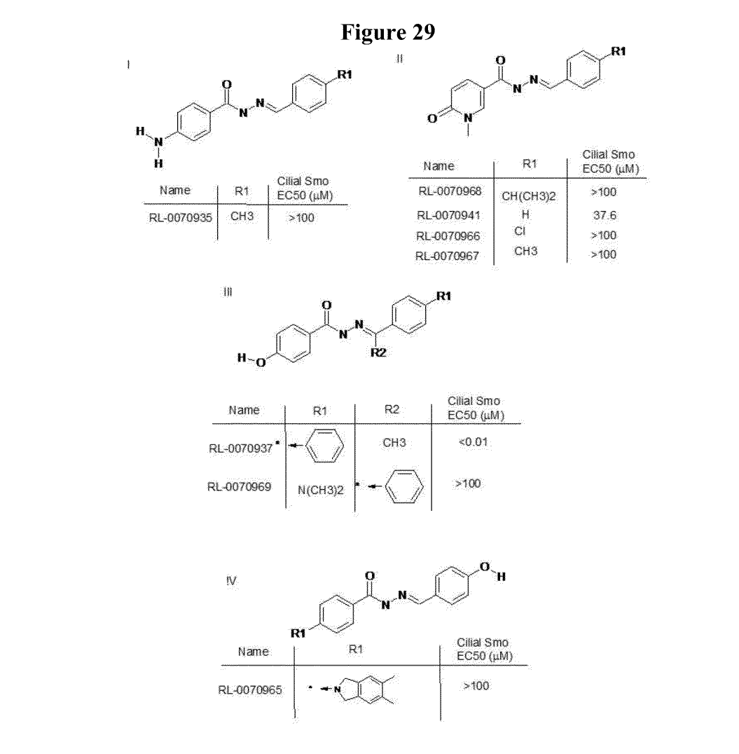

In addition, a very limited set of analogs (FIG. 29) were acquired for SAR exploration purposes which (1) substitute the hydroxy with a primary amine (Scheme I), (2) modify ring B (Scheme II), (3) modify the linkage between rings A & B (Scheme III), (4) reverse the position of the hydroxyl on the chemical scaffold (Scheme IV).

### REFERENCES

1. Zuercher, W. J. et al. Identification and structure-activity relationship of phenolic acyl hydrazones as selective agonists for the estrogen-related orphan nuclear receptors ERRbeta and ERRgamma. J Med Chem 48, 3107-9 (2005).

### Example 3

### Structure Activity Relationship Analyses of Inhibitory Activity of Smo Cilial Accumulation by SMANT and Analogs

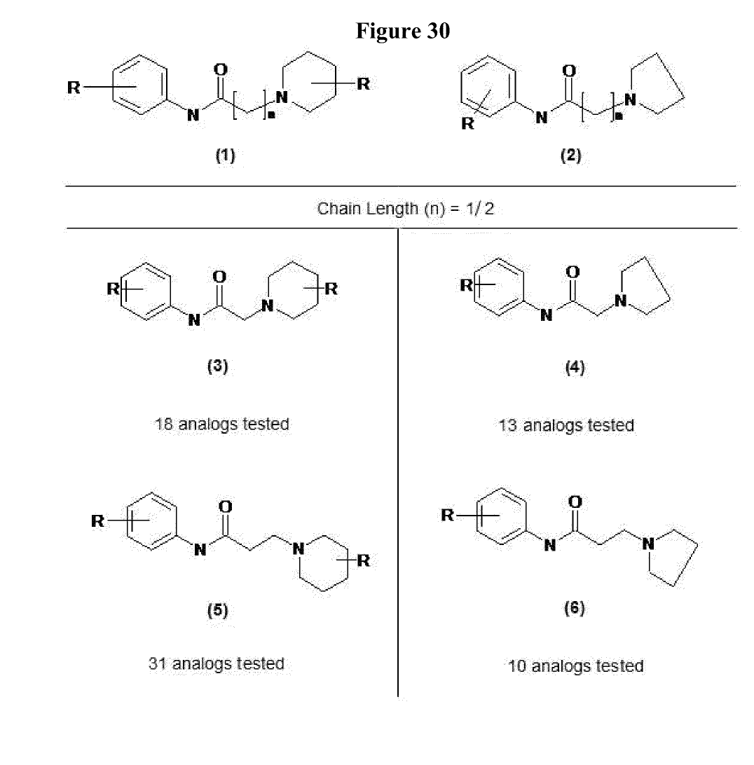

Inventors assembled a library around chemistry of SMANT/SMANT-2 consisting of 72 azacyclo-N-Alkylanilides (FIG. 30). The chemical series were differentiated by the chain length size (n=1 or 2) and the types of cyclic secondary amine. The two types of substituted cyclic secondary amines have piperidine (1) or pyrrolidine (2) moieties. The compounds where the chain length (n) is equal to 1 with either a piperidine (3) or pyrrolidine (4) have been confirmed as inactives in the Smo antagonist high content screen. However, Smo antagonist activity is confirmed when the chain length (n) is equal to 2 with either a piperidine (5) or pyrrolidine (6) moiety. The piperidine containing subclass of the azacyclo-N-alkylanilides series are more potent Smo antagonists compared to the pyrrolidine class. In our studies, we have elaborated upon the structure activity relationship (SAR) of this class for the inhibitory activity of Smo cilial localization induced by Sonic Hedgehog (Shh). We primed Smo::EGFP/Ivs::tagRFPT cells with compounds for 3 hours before adding Shh in order to increase assay sensitivity.

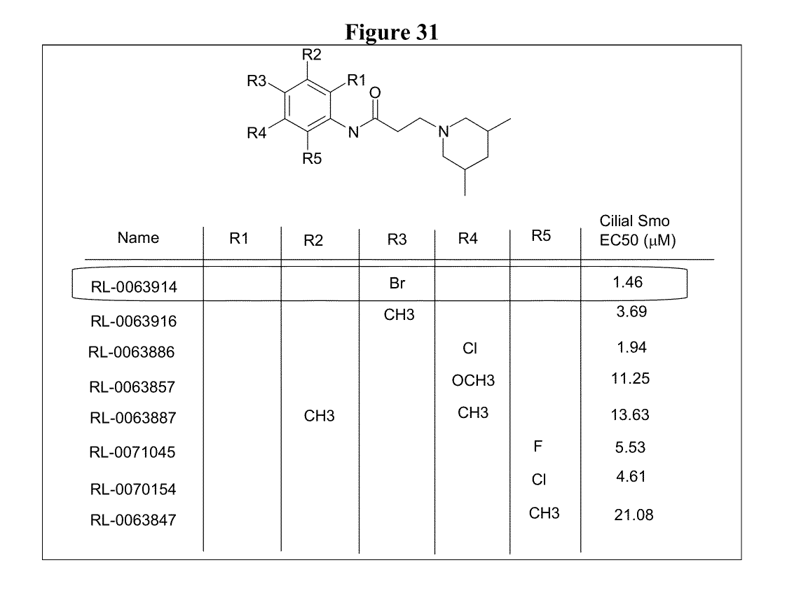

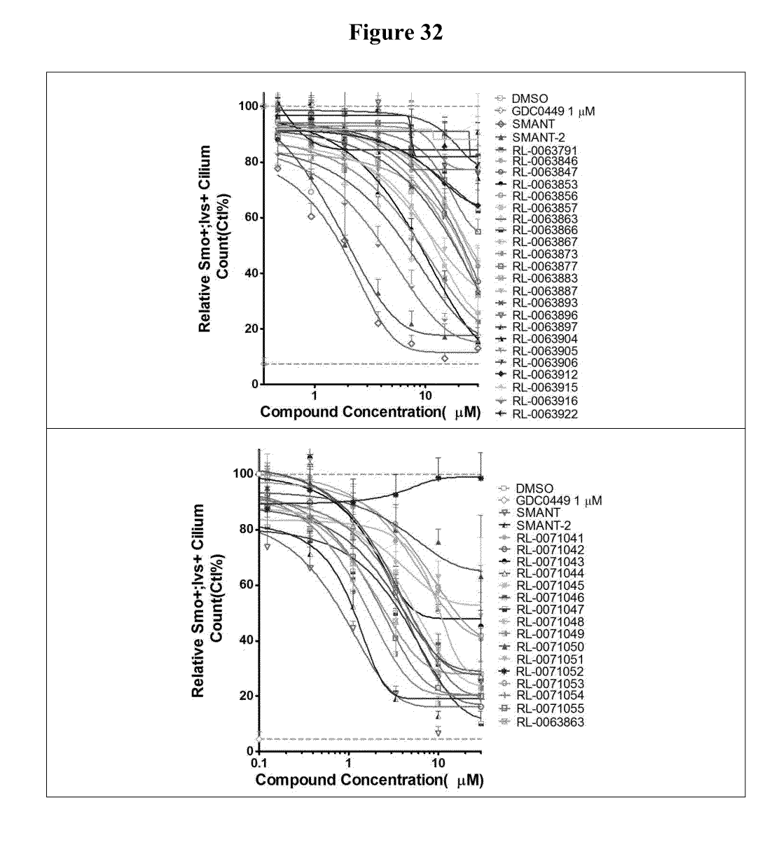

The most potent compounds identified was SMANT (assigned with catalog #RL-0063914 in house; EC50=1.46 μM). We first looked at a number of analogs of SMANT where the cyclic secondary amine remained constant to study activity effect by varying the substituted aniline (FIG. 31). FIG. 32 shows all of the dose-response curves generated during our experiments. Preliminary SAR data suggest that the location of the halogen or electron withdrawing group (EWG; o-, m-, p-substitution) is of importance to the compounds' activity. In general, the more potent Smo antagonists contain weak electron withdrawing groups (Br, Cl, F) compounds compared to compounds containing electron donating groups (OCH3, CH3).

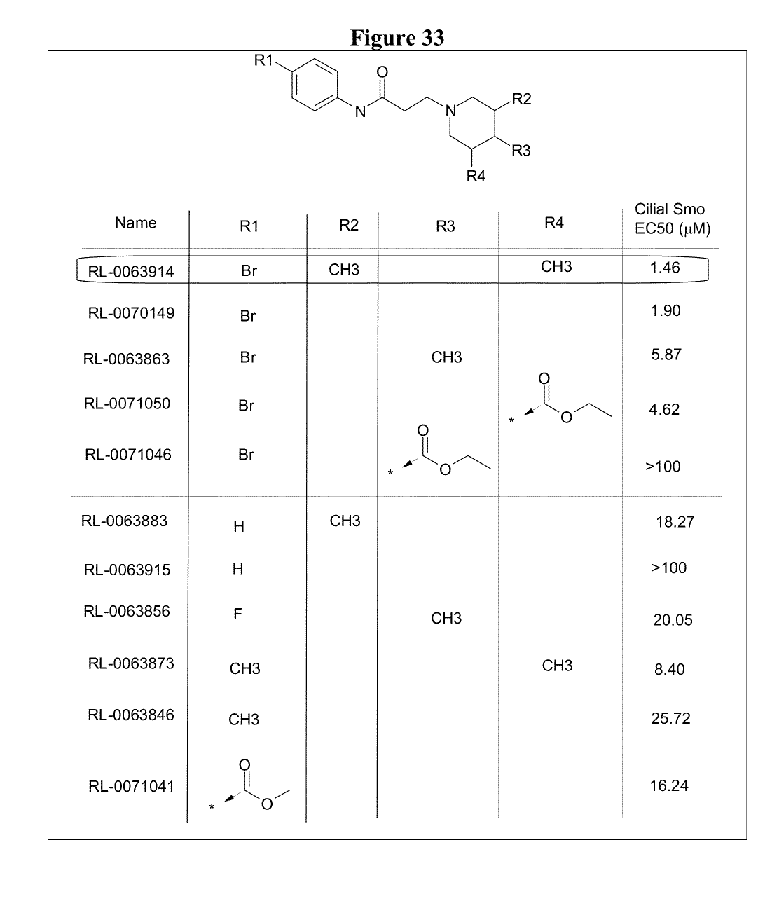

When grouping analogs with same substitution of the R1 position, preferably a EWG group, and varying substitution at the other ring (FIG. 33), we observed that substitutions in R2, R4 are more tolerated than R3 in order to retain Smo antagonist activity. The location of the substituent (R2 or R4>R3) on the cyclic amine moiety can be of importance when further developing the SAR of the series.

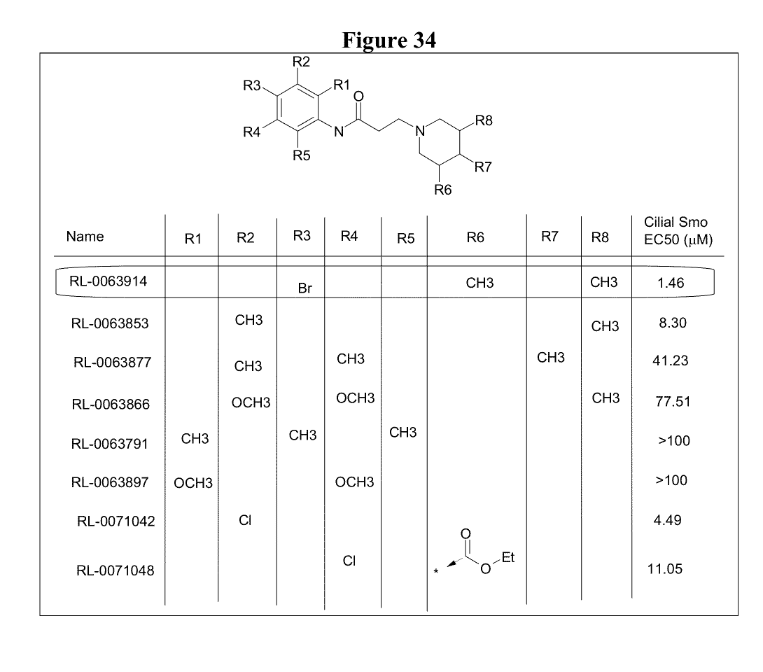

A selected few compounds have been tested where substituent positioning at both the aniline and piperidine is varied (FIG. 34). A number of the compounds (e.g. RL-0063791, RL-0063897, RL-0063877, and RL-0063866) are inactive when multiple electron donating groups are positioned at either end, or just the aniline side, of the molecule. Overall, the data supports pervious observations

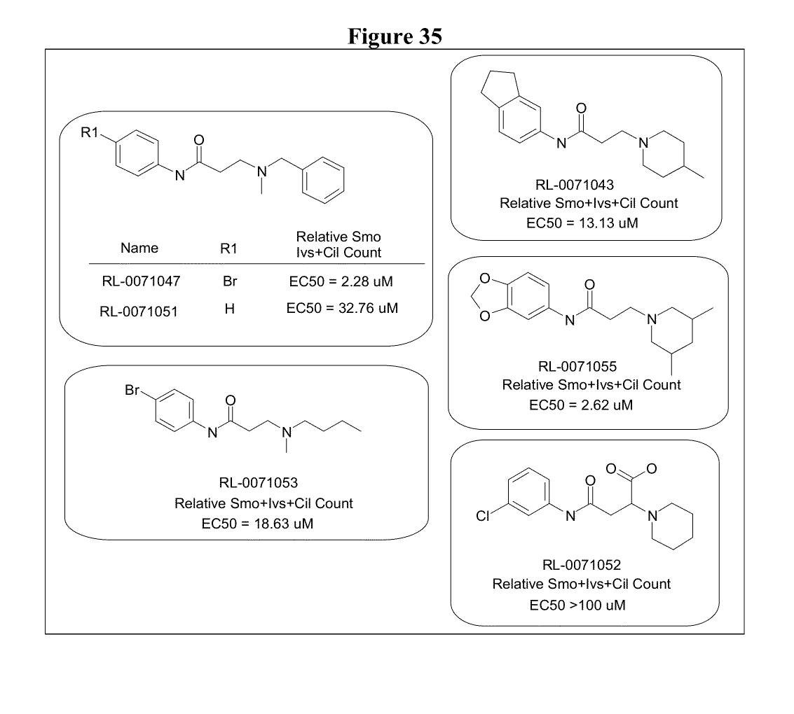

A few compounds were tested that expand or change the pyridine moiety to another group (FIG. 35). The most interesting result is RL-0071047 (EC50=2.28 μM) compared to RL-0071051 (EC50=32.76 μM) and RL-0071053 (EC50=18.63 μM). A more dramatic change of the secondary amine building block is tolerated when an appropriate functional group is kept on the aniline side of the molecule. The data on RL-0071043 and RL-0071055 shows possibilities of expanding to bicyclic groups with retained activity. Interestingly, the one branched compound (RL-0071052) inactivates Smo antagonist activity.

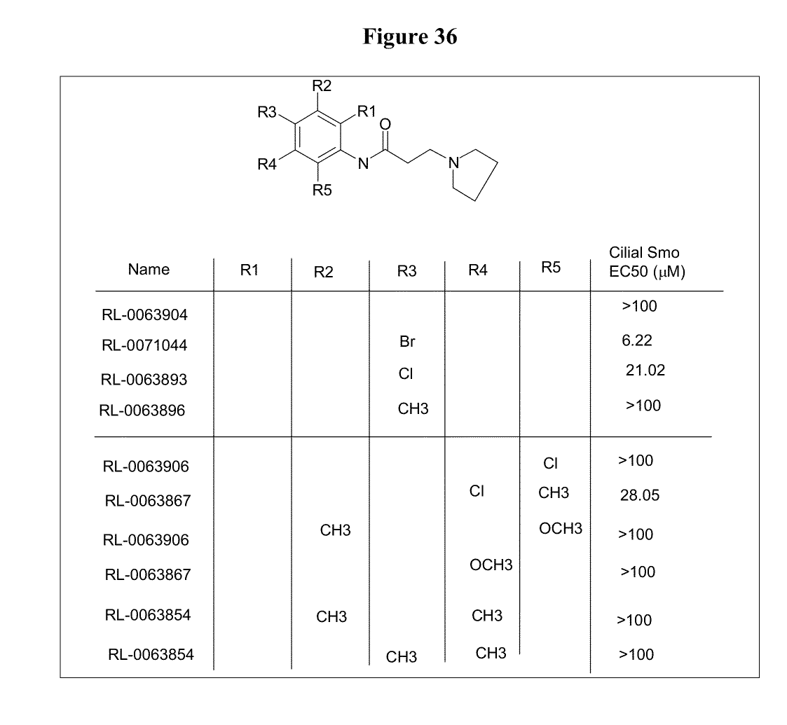

As discussed above, the piperidine containing subclass of the azacyclo-N-alkylanilides series are more potent Smo antagonists compared to the pyrrolidine class. FIG. 36 shows our results on the pyrrolidine class. The class is generally inactive because the lack of substituent's off of the pyrrolidine ring. However, RL-0071044 (EC50=6.22 μM) shows a good example where the R3 position contains bromine with retained, but weaker, Smo antagonistic activity compared to RL-0070149 (piperidine). Compound RL-0063904 is inactive, similarly to RL-0063915 (FIG. 33).

### Example 4

### Potential Glucocorticoid Drug Crosstalk with the Hedgehog Pathway

The Hedgehog (Hh) pathway is one of the central pathways of animal development, and deregulated pathway activity underlies a multitude of diseases, notably a variety of cancers¹. Activating mutations in Hh pathway components are cell intrinsic causal factors in cancers linked to Gorlin syndrome, medulloblastoma (MB), basal cell carcinoma (BCC), and rhabdomyosarcoma (RMS). In addition, paracrine Hh signaling-based modulation of the tumor microenvironment is thought to play a wider role in the support of a number of other malignancies including those of the breast, lung, liver, stomach, pancreas, prostate, and colon². Hh signaling is also linked to medically beneficial actions such as the promotion of stem/progenitor cell proliferation that may enable regenerative therapies. Considerable clinical interest has developed about the mechanisms of Hh pathway action and the identification of drugs that can modulate pathway activity.

Smoothened (Smo), a seven-pass transmembrane protein, has emerged as a predominant target in screens for small-molecule pathway modulators. Smo is essential for all Hh signaling³. All 7 drugs in clinical trials for Hh targeted cancer therapy act directly on Smo to inhibit Hh signaling⁴. Smo regulation is quite unusual. Hh binding to its receptor Patched-1 (Ptch1) counters Ptch1 mediated inhibition of Smo, enabling Smo-dependent activation of a Gli-based transcriptional response⁵. These events correlate with, and are critically linked to, the primary cilium (PC), a tubulin-based cell extension present on most vertebrate cells⁶. After binding Hh, Ptch1 moves from the PC while Smo accumulates on the ciliary axoneme. Though the mechanistic details are unclear, Smo action at the PC is essential for pathway activation^{7,8}, and this cellular translocation presents an opportunity for novel drug development.

In this work, the inventors report on a high content screen (HCS) to identify small molecules that modulate Smo accumulation at the PC. Most strikingly, the inventors identified a large number of glucocorticoids (GC), several of which are in clinical use, that induce this activity. Surprisingly, these compounds fail to trigger robust pathway activation; instead, they sensitize cells to Hh ligand input and impair pathway inhibition by co-administered pharmacological antagonists of Smo signaling. In contrast, anther steroid, Budesonide, inhibits Smo ciliary translocation and Hh signaling, synergizing with GDC0449, a Smo antagonist under clinical evaluation. Importantly, Budesonide acts similarly on wildtype Smo, and an oncogenic mutant form refractory to other Smo antagonists, SmoM2⁹. These findings have important ramifications for the design of new therapeutic approaches to treat cancers whose growth can be modulated by Smo activation, and potential implications for off-target crosstalk of glucocorticoid drugs in the Hedgehog signaling pathway.

### Materials and Methods

Cell Culture:

NIH/3T3 cells were maintained in DMEM containing 10% (v/v) calf serum, penicillin, streptomycin, and L-glutamine. HEK293, L, cos 7, and suFU−/− mouse embryonic fibroblast cells were maintained in DMEM containing 10% (v/v) fetal bovine serum, penicillin, streptomycin, and L-glutamine. Smo::EGFP and Ivs:: tagRFPT were cloned into pBabe retroviral constructs. Smo::EGFP/Ivs::tagREPT stable cell lines was generated through viral infecting NIH/3T3 cells according to the procedure described previously¹⁰. A ShhLightII cell line (ATCC) was used for Gli-luciferase reporter assays. This line contains a stably integrated Gli-responsive firefly luciferase reporter and a constitutive Renilla luciferase expression construct⁴³. A subclone of this cell line was created expressing a stably integrated SmoM2 expression construct. Shh conditioned medium was collected from cos 7 cells transfected with an expression construct encoding the amino terminal 19 kDa signaling peptide of Shh and used at 13.7(±3.0) nM unless stated otherwise. Control conditioned medium was collected from cos 7 cells transfected with an empty plasmid. Wnt3a conditioned medium was collected from an L cell line stably expressing a Wnt3a expression construct. Control conditioned medium was collected from wild-type L cells. All conditioned medium were diluted 1:10 prior to assay.

Reagents:

Chemical libraries screening utilized the Library of Pharmacologically Active Compounds (LOPAC, Sigma-Aldrich), the Spectrum Collection (Microsource Discovery Systems), and the Prestwick Chemical Library (Prestwick Chemical), along with a custom collection of additional biologically annotated chemistries absent from the above pre-plated reference collections. Glucocorticoids, cyclopamine, and forskolin for follow-up studies were purchased from Sigma. SAG was purchased from Axxora Platform. SANT-1 was obtained from Tocris Biosciences. GDC0449 was purchased from Selleck Chemicals. BODIPY-cyclopamine was purchased from Toronto Research Chemicals. All small molecule stock solutions were prepared by dissolving in DMSO at 1 or 10 mM and stored at −20° C. Mouse recombinant ShhN purified protein (IIShhN) was a gift from Dr. Pepinsky (Biogen Inc). Transfection was performed using Fugene6 or Fugene HD (Roche).

Imaging Assays:

Cells were cultured and treated in 384-well imaging plate precoated with poly-D-Lysine (Greiner Bio-one), fixed with 4% paraformaldehyde (Electron Microscopy Sciences), and stained with Hoechst (Invitrogen). Images were collected using Opera High Content Screening System (Perkin Elmer). ActivityBase (IDBS), Pipeline Pilot (Accelrys), Excel (Microsoft), and Prism (GraphPad) were used for high content screening data management and analysis. Acapella 2.0 software (Evotec Technologies/PerkinElmer) was used to perform multi-parametric image quantification. All the comparative images were scanned with identical microscopic setting and analyzed with the same input parameters.

Hh and Wnt Activity Assays:

ShhLightII cells and SmoM2/LightII cells were cultured and treated in 96 well assay plates (Corning) and incubated with Duo-Glo luciferase substrates (Promega) to sequentially measure firefly and renilla luciferase activity. Smo, or GFP, expression plasmids were co-transfected into 3T3 cells together with a Gli-responsive firefly reporter and a TK-renilla luciferase reporter contruct to monitor effects of Smo overexpression. Co-transfection of the two reporter constructs was conducted in assays measuring Hh pathway activity in suFU−/− cells. Wnt activity was measured following co-transfection of a Top-flash and renilla luciferase reporter. In both Hh and Wnt activity assays, renilla luciferase reporter activity, or mass of protein, was used to normalize expression values. Luciferase signal was read by TopCount NX Microplate Scintillation and Luminescence Counter (Perkin Elmer). Quantitative PCR probes for Ptch1, Gli1, and β-actin were purchased from Applied Biosystems. Reactions and measurements were performed using on an Applied Biosystems 7900HT at Harvard FAS Center of System Biology. β-actin was used to normalize Ptch1 and Gil1 values.

Bodipy-Cyclopamine Competition Assays:

Cos 7 cells were transfected with a plasmid that co-expresses Smo and a nuclear localized tagRFPT marker (pCIT-Smo). The empty parental construct (pCIT) and a construct that co-express SmoM2 were used as controls to assess specificity and background signal. Three days after transfection, cells were incubated with 5 nM Bodipy-cyclopamine, with or without additional compounds, for 1 hour at 37° C. Cells were then fixed and stained with Hoechst. Images were collected with the Opera High Content Screen System. Fluorescence values were assessed in transfected cells (red nuclei) with a program developed by the authors using Acapella 2.0 software. All of images were scanned with identical microscopic setting and analyzed with the same input parameters.

CGNP Proliferation Assays:

CGNP primary cells were isolated from P7 Ptch1+/− mice as previously reported⁴⁴. Cells were seeded in poly-D-lysine coated imaging plates (Greiner Bio-one), treatments were applied 2 hours thereafter and last for 36 hours. Cells then were fixed with 4% paraformaldehyde (Electron Microscopy Sciences), and stained with anti-pH3 antibody (Upstate; 1:100) followed by a secondary antibody (Invitrogen) and Hoechst (Invitrogen). Images were collected and cell proliferation quantified with a program developed by the authors utilizing Acapella 2.0 software. All of the images in each experiment were collected with identical microscopic settings and analyzed with identical input parameters.

### Results and Discussion

Development of a High Content Screen to Identify Agonists of Smo Ciliary Accumulation.

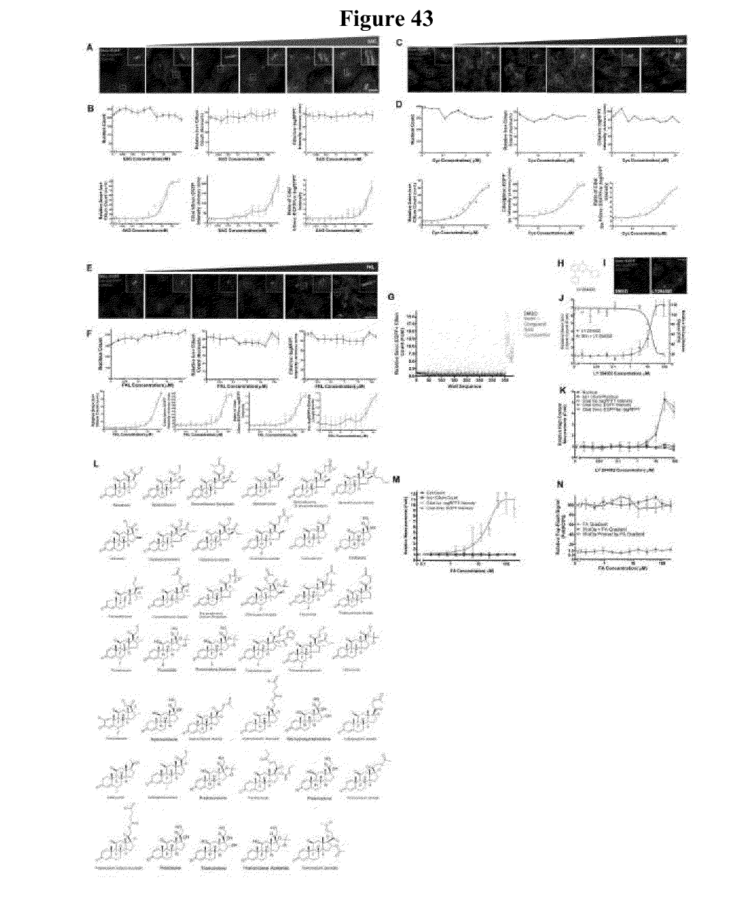

To gain a more comprehensive view of the Hh pathway at early stages of drug development, we developed and validated a novel High Content Screening (HCS) method based directly on Smo translocation to the PC. An EGFP tagged form of human Smo was introduced into Hh responsive NIH3T3 cells¹⁰ (FIG. 43 A) to generate a clonal cell line in which Hh-dependent accumulation of Smo::EGFP in the PC mirrored movement of endogenous Smo¹⁰. An Inversin(Ivs)::tagRFPT expression cassette provided a constitutive, independent PC marker¹¹.

Custom algorithms were developed to perform quantitative multi-parametric image analyses. Robust dose dependent responses were observed upon treatment with several known small molecule modulators of Smo: the agonist SAG and the antagonist cyclopamine (Cyc), both of which directly bind Smo, and forskolin (FKL), whose stimulatory action on protein kinase A inhibits Smo signaling (FIG. 43 A-F). Despite of antagonist activity, Cyc, as the same as SAG, physically interacts with Smo and induces its accumulation in the PC, which suggests that Smo accumulation may be necessary but not sufficient for the pathway activation. FKL, on the other hand, may induce Smo ciliary accumulation indirectly through acceleration of anterograde intraflagellar transport. The divergence of differential modulations of Smo ciliary translocation by these agents suggest complicated mechanisms of this critical process in the Hh pathway and highlight the need for a better understanding. As a demonstration of the assay's ability to detect local changes within the PC, elongation of the PC on FKL treatment was detected as an expanded Ivs+ domain (last panel in FIG. 43F), consistent with a recent report¹².

Screening Results.

We conducted a screen with a library consisting of 5,672 compounds with annotated activities, including FDA approved drugs and drug candidates in preclinical or clinical development. Representative examples of plates including small-molecule control wells are shown for the analysis (FIG. 43G). Z-prime scores¹³ consistently >0.4 indicate the statistical robustness of the primary screen.

Approximately 60 compounds in 15 distinct chemical classes were confirmed to induce Smo accumulation at the PC, after rigorous assessment of the dose-response curves for primary hits. As expected, these comprised both pathway agonists and antagonists. For example, LY 294002, an inhibitor of phosphatidylinositol 3-kinase (PI3K)¹⁴, induces Smo ciliary accumulation, but inhibits Hh signaling (FIG. 43H-K). The PI3K pathway is important in a variety of cancer types and may intersect with the Hh pathway in tumorigenesis¹⁵. When combined, a PI3K inhibitor and a Smo antagonist induce a delayed onset of drug resistance in a mouse model of medulloblastoma¹⁶. PI3K action has been linked to the regulation of Gli2¹⁷. These data suggest that PI3K may act at multiple levels in Hh signaling.

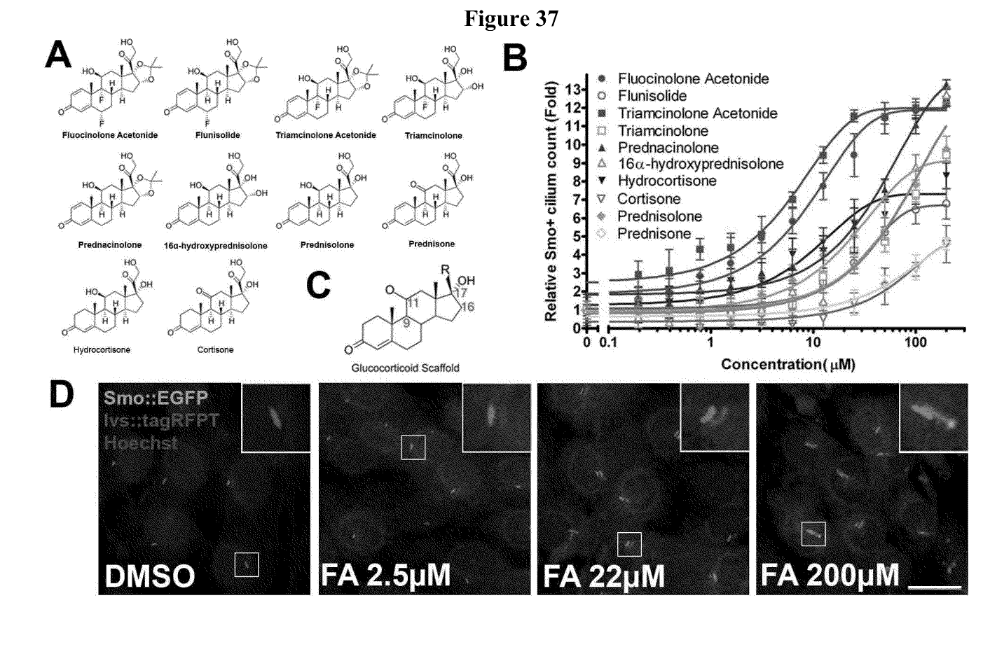

Strikingly, the most predominant chemical class identified comprised naturally occurring and synthetic glucocorticoids (GC), several of which are widely used as anti-inflammatory agents in the clinic (FIG. 37A-B; FIG. 43L)¹⁸. A recently screen examining β-arrestin aggregation identified a subset of these compounds, lending additional support to a GC intersection in Smo-directed Hedgehog signaling¹⁹. Structure-activity relationship (SAR) analysis suggests that fluorine at position 9, a ketal at positions 16 and 17, and protonation at position 11 significantly enhance the potency of this class of compounds in directing Smo accumulation to the PC (FIG. 37C).

GCs Accumulate Smo in the PC without Activating the Hh Pathway.

To investigate in more detail the consequences of GC-induced Smo accumulation in the PC, and to obtain mechanistic insights into GC action in the Hh pathway, we chose one compound in clinical use, fluocinolone acetonide (FA). FA displays an EC50 of around 5 μM for accumulation of Smo in the PC; in addition, no obvious cytotoxic effects are observed in vitro at much higher doses (up to 200 μM; FIGS. 37B and D, FIG. 43M). Localization of an inversin-based PC reporter and other PC markers including Arl13b and acetylated tubulin were unaltered in response to FA (FIG. 37D; FIG. 43M; data not shown). Further, no change was detected in the activity of a Wnt-signaling reporter in response to FA concentrations that modify Smo distribution (FIG. 43M-N). Together these data suggest that FA's effects in this assay are specific to the Hh pathway.

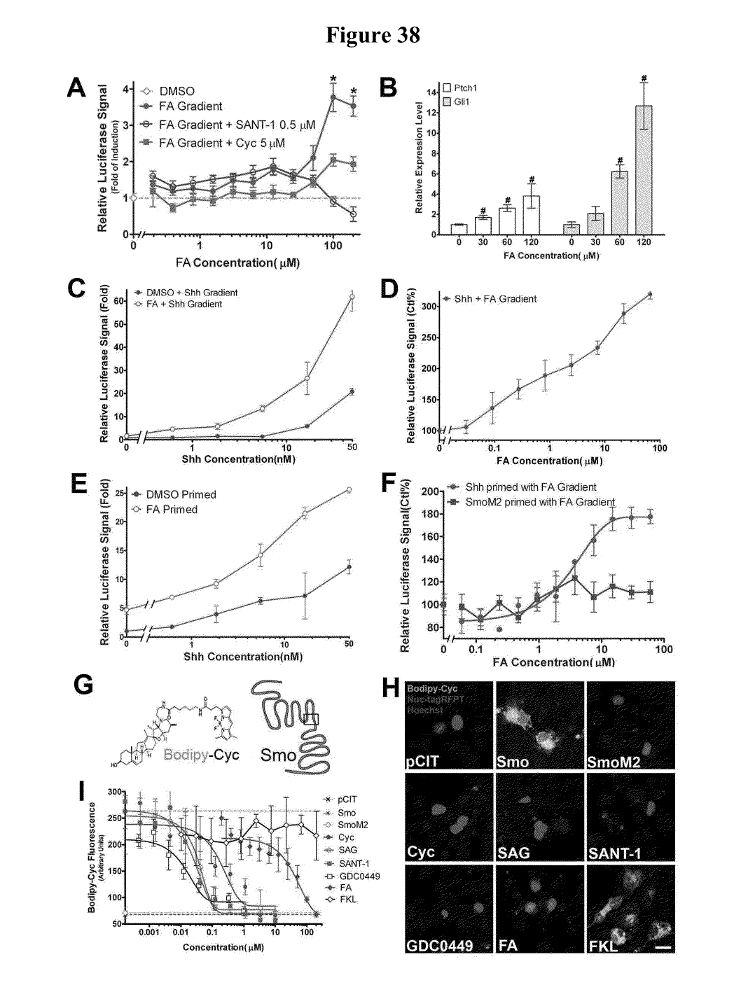

The accumulation of Smo in the PC is thought to be essential for transcriptional activation of the Hh pathway^{5,20}. However, we observed a marked disparity between FA-induced Smo accumulation in the PC and Hh pathway activation in transcription reporter assays. At low levels of FA that effectively promote Smo accumulation in the PC (10 μM), no pathway activation was observed. Higher concentrations (>50 μM) invoked a weak transcriptional response measurable in a Gli-luciferase reporter assay (4 fold versus 25 fold for 1 μM of the Smo agonist SAG in the same assay (data not shown)), and on quantitative reverse transcription-polymerase chain reaction (qRT-PCR) measurement of Hedgehog target gene expression (Ptch1 and Gli1; FIG. 38A-B). The EC50 for weak transcriptional activation (>50 μM) was 10 fold higher than that of FA-induced accumulation of Smo within the PC.

FA Induces Hypersensitivity to Hh Pathway Stimulation.

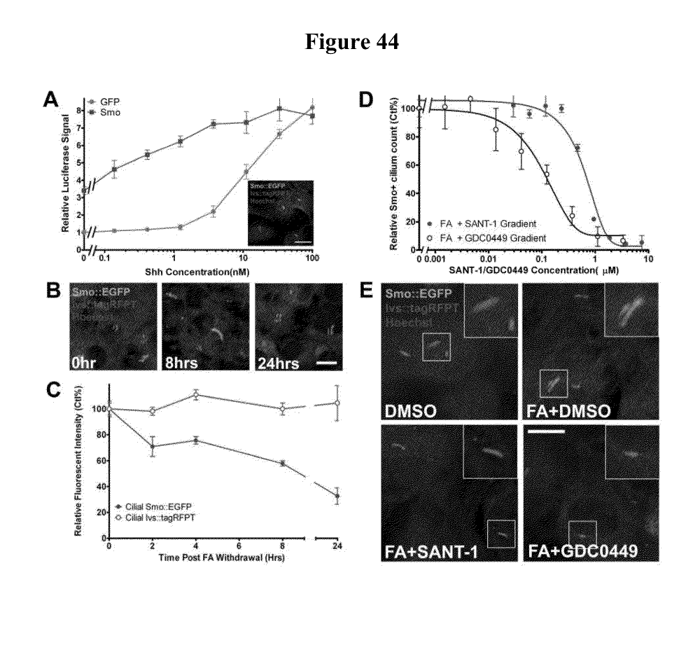

The effects of FA resemble over-expression of Smo in that constitutive accumulation of wild-type Smo within the PC only results in weak pathway activation (FIG. 44A). Ciliary accumulation of Smo sensitizes cells to subsequent Sonic hedgehog (Shh) ligand input, raising the possibility that FA-driven Smo accumulation may sensitize Hh responsive cells. Indeed, co-stimulation of cells with 10 μM FA results in a dose-dependent enhancement of a Shh-induced transcriptional response (FIG. 38C-D). Furthermore, this effect was measurable after prolonged withdrawal of FA; cells treated for 24 hours with FA followed by compound withdrawal prior to Shh addition showed a higher induction of pathway activity than DMSO treated controls (FIG. 38E-F). The EC50 of a FA induced response to priming is approximately 4 μM, in good agreement with the dose required for efficient accumulation of Smo in the PC (FIG. 37B). Smo turnover in the PC is relatively slow after Shh-invoked pathway activation¹⁰, or compound withdrawal (FIG. 44B-C), providing a potential explanation for a FA induced pathway priming effect. FA treatment showed no effect on Wnt pathway activity (FIG. 43N), consistent with Hh pathway specificity.

FA May Regulate Smo by Direct Binding.

To determine whether FA interacts with Smo, we performed a competition assay with Bodipy-Cyc. Cyc binds Smo directly²¹ and its fluorescent analog, Bodipy-Cyc, shows strong Smo-dependent fluorescence within cells over-producing Smo (identified by co-expression of a nuclear localized tagRFP-T, FIG. 38G-H). An oncogenic mutation within the 7^{th} transmembrane domain (SmoM2, also named SmoA1, FIG. 38H-I)²¹, and a recently described drug resistance mutation within the 6^{th} transmembrane domain (SMOD473H) significantly impair Cyc binding to Smo, suggesting that these are critical sites for chemical interaction²².

FA displayed a dose-dependent competition of Bodipy-Cyc binding to wild-type Smo, similar to other small molecules that directly bind Smo (SAG, and GDC0449), or that likely interact directly with Smo based on similar competition assays (SANT-1) (FIG. 38H-I)²¹⁻²⁴. In contrast, FKL induces Smo accumulation in the PC but does not compete with Bodipy-Cyc, reflecting an indirect action through its protein kinase A target^{25,26}. FA induced activation of the Hh pathway at high concentrations is blocked by Cyc and SANT-1 (FIG. 38A), and SANT-1 and GDC0449 inhibit FA promoted accumulation of Smo in the PC (FIG. 43D-E). Collectively, these data support a direct interaction between FA and Smo.

Antagonistic Drug-Drug Interactions Between FA and Smo Antagonists.

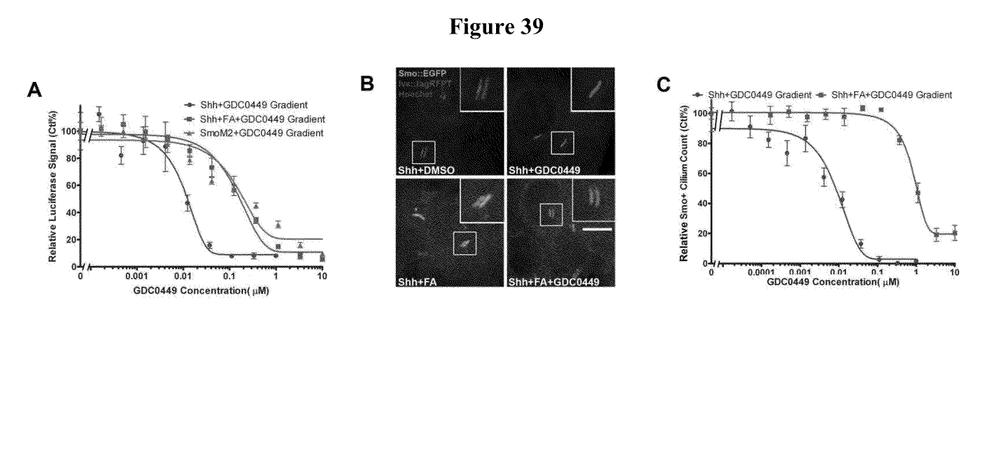

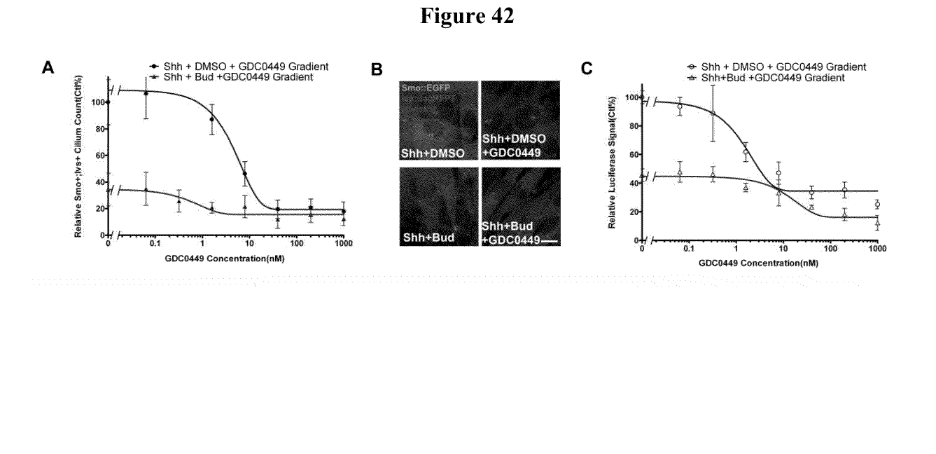

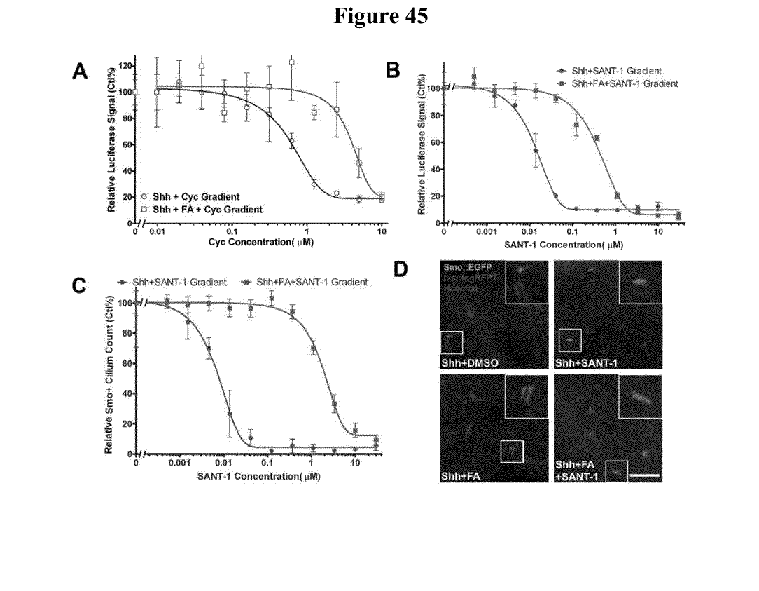

Considering that GCs and various Hh pathway antagonists may share a common Smo target, and GCs are widely used to suppress inflammation in conjunction with cancer therapy, we next asked whether we could observe a potential GC crosstalk with Smo antagonists in cell culture assays. Hh pathway inhibition by GDC0449, Cyc and SANT-1, as measured by both Gli-luciferase induction (FIG. 39A and FIG. 42) and Smo ciliary localization (FIG. 39B-C and FIG. 45), was dramatically reduced in vitro in the presence of FA. Thus, FA co-treatment leads to a drug-dependent impairment of cellular response to chemical inhibitors of Smo. This may occur through competition, or another mechanism, but the outcome resembles the genetic resistance seen with a dominant active Smo mutation (SmoM2) (FIG. 39A).

Ex Vivo Studies of FA with Ptch1+/−CGNPs.

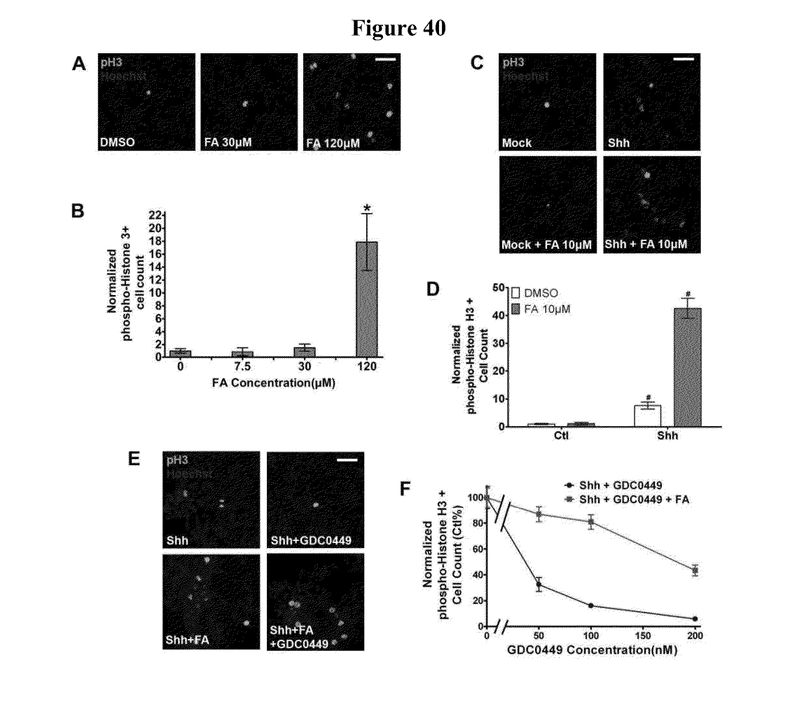

To further explore FA actions, we isolated cerebellar granule neuron precursors (CGNPs) from Ptch1+/− neonates. Proliferation of CGNP is Shh-dependent and Ptch1 heterozygosity predisposes both mice and humans to develop CGNP-derived medulloblastoma²⁷. Consistent with results on Hh pathway activation in NIH3T3 cells, only very high doses of FA (120 μM) elevated the number of proliferative, phospho-histone H3 (pH3) positive GCNPs (FIG. 40A-B). However, a lower dose of FA (10 μM) markedly enhanced Shh-driven CGNP proliferation (FIG. 40C-D). Further, co-administration of FA (10 μM), with the Smo antagonist GDC0449, impaired GDC0449 inhibition of Shh-stimulated GCNP proliferation (FIG. 40E-F).

GC Inhibitors of Smo Accumulation to the PC and of Smo Signaling.

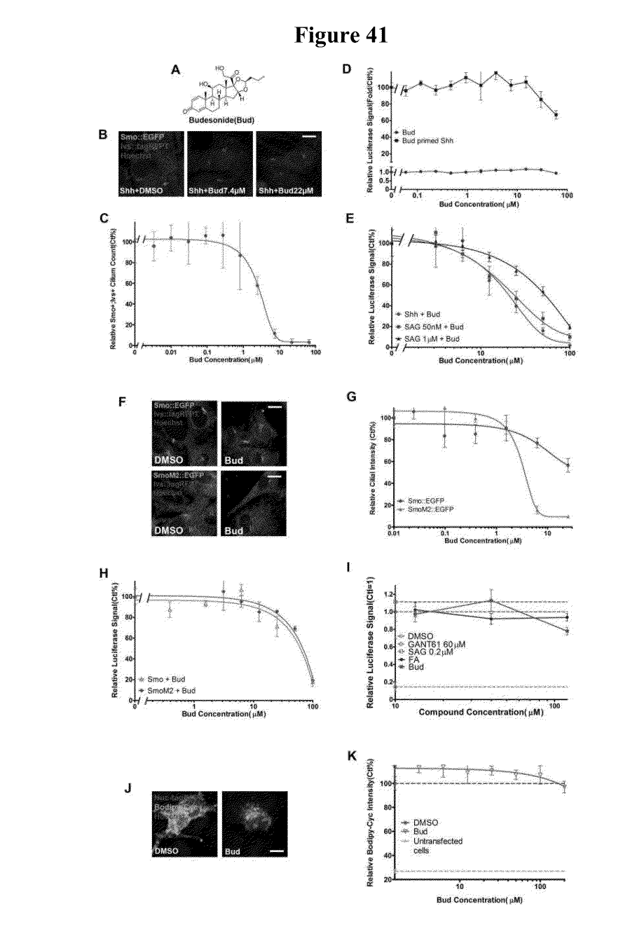

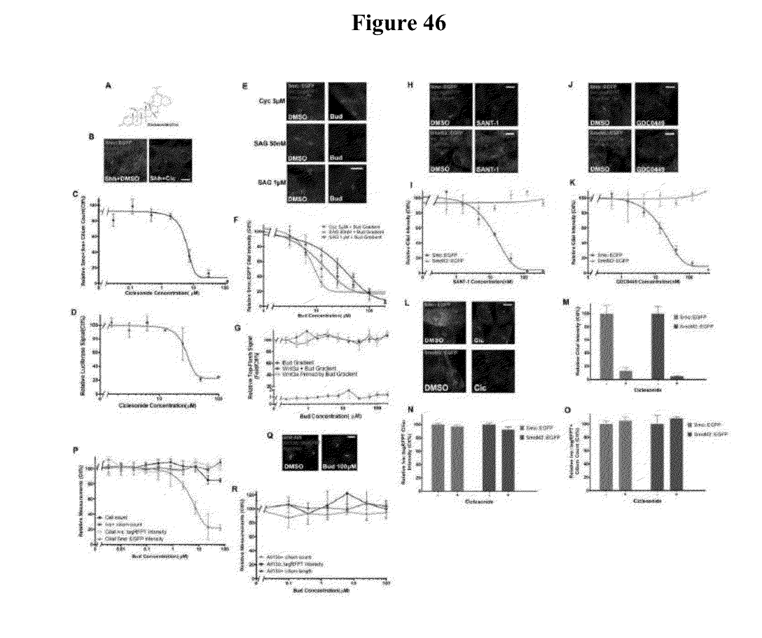

While a large number of GCs promote Smo ciliary accumulation, secondary assays of small molecules sharing the core GC scaffold identified two inhibitory GCs: Budesonide (Bud) and Ciclesonide (Cic) (FIG. 41A-C; FIG. 46A-C). When compared with Smo promoting GCs, Bud and Cic are distinguished by bulky hydrophobic groups at positions 16 and 17 (FIG. 37; FIG. 43L; FIG. 41A; FIG. 46A). In contrast to FA, Bud had no pathway inducing activity, nor did Bud induce a hypersensitive response to Hh ligand (FIG. 38A-F; FIG. 41D), reinforcing the association of hyper-responsiveness to Smo ciliary accumulation activity. As expected from the inhibition of Smo accumulation in the PC, Bud and Cic inhibited Shh dependent activation of a Gli-reporter (FIG. 41E and FIG. 46D). Further, Bud attenuated Smo ciliary accumulation and pathway activation by SAG (FIG. 41E; FIG. 46E-F), and also suppressed Cyc induced Smo accumulation to the PC (FIG. 46E-F). Bud treatment showed no effect on Wnt pathway activity (FIG. 46G), consistent with a specific modulation of Hh signaling outside of its GC activity.

Bud and Cic Inhibit SmoM2 Ciliary Localization and its Signaling.

SmoM2 encodes a dominant active Smo variant identified in a human cancer that is resistant to inhibition by available Smo antagonists at concentrations that completely suppressed wildtype Smo activity (FIG. 46H-K). Unexpectedly, both Bud and Cic attenuated SmoM2 ciliary localization, and downstream pathway activity, as effectively as wildtype Smo (FIG. 41F-H; FIG. 46L-M). Bud and Cic did not disrupt ciliary structure or ciliary trafficking: acetylated-tubulin (acet-tub), Ivs::tagRFPT, and Arl13b::tagRFPT within the PC were unaltered on treatment (FIG. 46N-R). To examine the site of Bud action in the Hh pathway, we examined Hh signaling activity following removal of suppressor of Fused (suFU) activity, a Gli repressor functioning downstream of Smo. Distinct from GANT61²⁸, Bud failed to suppress ligand-independent Hh pathway activity induced by loss of suFU function (FIG. 41I). Together these data suggest that Bud may act at the level of Smo but through a different mechanism than other Smo-interacting antagonists including SANT-1, Cyc, and GDC0449, and also distinct from FA and SAG. Consistent with a unique inhibitory action, Bud failed to compete with Bodipy-Cyc even at levels well above the inhibitory maximum (100 μM; FIG. 28J-K). Further, whereas FA competed with GDC0449 to suppress effective pathway inhibition (FIG. 39), Bud enhanced GDC0449's activity to block Smo accumulation at the PC and Hh pathway inhibition (FIG. 42).

The interaction of GCs with the Hh pathway leads to several important observations: First, all small molecules that induce ligand-independent Smo accumulation to the PC characterized to date either activate or inhibit Smo activity. Agonists include SAG and purmorphamine^{23,24,29}. Cyc though an antagonist also induces Smo transolcation to the PC^{10,26,30}. Several lines of evidence indicate that whereas Smo accumulation in the PC is essential for signaling, accumulation is not sufficient, with additional ligand-dependent actions being required to generate an active form of Smo^{10,11,26,30}. Together, our data suggest that many GCs can function in a novel mechanism that synergizes with Hh-ligand-directed signaling by promoting accumulation of Smo within the primary cilium. Such synergistic effect might be due to bypassing the Ptch1-mediated “barrier” for Smo entry to the primary cilium and priming the cells for activation of Smo within the organelle.

Second, the finding of a potential effect of Smo promoting GCs in modulating the Hh response highlights the value of a “direct target screen” focusing on critical parameters of target action. To date most small molecule Hh pathway modulators have been identified through “end-point” transcriptional assays. However, because of their modest effects on transcription, GC interactions are not readily detected with this screening approach.

Third, the dose of GC required to modify Smo localization (EC50s>1 μM) is significantly higher than that required to directly modulate GC receptor-based transcriptional responses (EC50s <10 nM or lower³¹). Thus, we believe GC's likely act directly on Smo at high concentrations, and not indirectly through a nuclear hormone receptor triggered transcriptional regulatory action.

Fourth, naturally occurring hydrocortisone and cortisone show different potencies in accumulating Smo to the PC (FIGS. 37A and B). 11β-hydroxysteroid dehydrogenase type 2 (HSD11β2), an enzyme that transforms hydrocortisone into cortisone, is up-regulated by Hh signaling in CGNPs³², whereas HSD11β1, an enzyme that mainly catalyzes the reverse reaction, was recently discovered as a target gene for Hh signaling in prostate cancer tissue³³. Taken together, these findings suggest potential feedback mechanisms linking the Hh transcriptional output to steroid regulation of Smo action.

Fifth, inflammation and cancer are linked, necessitating combinatorial therapies to treat both aspects of disease³⁴. To this end, GCs are frequently co-administered to patients receiving anti-cancer therapies. However, GCs are reported to enhance cancers of the breast³⁵, colon³⁶, lung³⁷, ovary^{35,38}, and pancreas³⁹, and to increase the metastatic potential of breast cancer⁴⁰. Amongst these are glucocorticoids that promote Smo ciliary accumulation in the current study. Further, FA is reported to act as a tumor promoter in the skin⁴¹. Our studies also raise the possibility of high dosing of glucocorticoids leading to off-target action of glucocorticoid agents in the Hh pathway, modifying therapeutic outcome: for example, in Hh-antagonist-directed cancer therapy. Whether an effective dose for GC drug-mediated crosstalk is reached during therapeutic administration is not clear, but pharmacokinetics of certain GC drugs in human patients warrants further investigation in a clinical setting. For example, the peak plasma concentration of Dexamethasone, a broadly used GC in cancer patients, can be as high as >10 μM after a single high dose⁴², which falls in the range where significant Smo cilial accumulation occurs (data not shown). Long-term systematic treatment, common in cancer therapy, might result in longer exposure to higher concentrations.

Sixth, our data indicates that most GCs likely share a similar interaction site with a broad range of agonists and antagonists including SAG, GDC0449, SANT-1, and Cyc, or modify Smo on binding to block access to this binding region. In contrast, Bud-like GCs do not compete with other Smo antagonists. Further, Bud works equally well inhibiting wildtype Smo, or a mutant form of Smo refractory to clinically active inhibitory compounds. Thus, it may act more like an allosteric regulator of Smo activity. Collectively, our findings highlight the potential to develop new drugs around a GC scaffold that may synergize with compounds currently undergoing clinical development to enhance anti-Hh-based cancer therapies and may also reveal more about the ways in which Smo trafficking and activity are regulated.

### REFERENCES

1. Rubin, L. L. & de Sauvage, F. J. Targeting the Hedgehog pathway in cancer. Nature Reviews Drug discovery 5, 1026-33 (2006).
2. Yauch, R. L. et al. A paracrine requirement for hedgehog signalling in cancer. Nature 455, 406-10 (2008).
3. Zhang, X. M., Ramalho-Santos, M. & McMahon, A. P. Smoothened mutants reveal redundant roles for Shh and Ihh signaling including regulation of L/R symmetry by the mouse node. Cell 106, 781-92 (2001).
4. Tremblay, M. R., McGovern, K., Read, M. A. & Castro, A. C. New developments in the discovery of small molecule Hedgehog pathway antagonists. Curr Opin Chem Biol 14, 428-35 (2010).
5. Rohatgi, R., Milenkovic, L. & Scott, M. P. Patchedl regulates hedgehog signaling at the primary cilium. Science 317, 372-6 (2007).
6. Goetz, S. C. & Anderson, K. V. The primary cilium: a signalling centre during vertebrate development. Nat Rev Genet. 11, 331-44 (2010).
7. Han, Y. et al. Dual and opposing roles of primary cilia in medulloblastoma development. Nat Med (2009).
8. Wong, S. et al. Primary cilia can both mediate and suppress Hedgehog pathway-dependent tumorigenesis. Nat Med (2009).
9. Xie, J. et al. Activating Smoothened mutations in sporadic basal-cell carcinoma. Nature 391, 90-2 (1998).
10. Wang, Y., Zhou, Z., Walsh, C. & McMahon, A. Selective translocation of intracellular Smoothened to the primary cilium in response to Hedgehog pathway modulation. Proc Natl Acad Sci USA (2009).
11. Watanabe, D. et al. The left-right determinant Inversin is a component of node monocilia and other 9+0 cilia. Development 130, 1725-34 (2003).
12. Besschetnova, T. Y. et al. Identification of signaling pathways regulating primary cilium length and flow-mediated adaptation. Curr Biol 20, 182-7 (2010).
13. Zhang, J. H., Chung, T.D. Y. & Oldenburg, K. R. A simple statistical parameter for use in evaluation and validation of high throughput screening assays. Journal of Biomolecular Screening 4, 67-73 (1999).
14. Vlahos, C. J., Matter, W. F. & Brown, R. F. A Specific Inhibitor of Phosphatidylinositol 3-Kinase. Journal of Cellular Biochemistry, 274-274 (1994).
15. Hambardzumyan, D. et al. PI3K pathway regulates survival of cancer stem cells residing in the perivascular niche following radiation in medulloblastoma in vivo. Genes & Development 22, 436-448 (2008).
16. Buonamici, S. et al. Interfering with resistance to smoothened antagonists by inhibition of the PI3K pathway in medulloblastoma. Sci Transl Med 2, 51ra70 (2010).
17. Riobo, N. A., Lu, K., Ai, X. B., Haines, G. M. & Emerson, C. P. Phosphoinositide 3-kinase and Akt are essential for Sonic Hedgehog signaling. Proceedings of the National Academy of Sciences of the United States of America 103, 4505-4510 (2006).
18. Sommer, P. & Ray, D. W. Novel therapeutic agents targeting the glucocorticoid receptor for inflammation and cancer. Current Opinion in Investigational Drugs 9, 1070-1077 (2008).
19. Wang, J. et al. Identification of select glucocorticoids as Smoothened agonists: potential utility for regenerative medicine. Proc Natl Acad Sci USA 107, 9323-8 (2010).
20. Kim, J., Kato, M. & Beachy, P. Gli2 trafficking links Hedgehog-dependent activation of Smoothened in the primary cilium to transcriptional activation in the nucleus. Proc Natl Acad Sci USA (2009).
21. Chen, J. K., Taipale, J., Cooper, M. K. & Beachy, P A Inhibition of Hedgehog signaling by direct binding of cyclopamine to Smoothened. Genes Dev 16, 2743-8 (2002).
22. Yauch, R. et al. Smoothened Mutation Confers Resistance to a Hedgehog Pathway Inhibitor in Medulloblastoma. Science (2009).
23. Frank-Kamenetsky, M. et al. Small-molecule modulators of Hedgehog signaling: identification and characterization of Smoothened agonists and antagonists. J Biol 1, 10 (2002).
24. Chen, J. K., Taipale, J., Young, K. E., Maiti, T. & Beachy, P. A. Small molecule modulation of Smoothened activity. Proc Natl Acad Sci USA 99, 14071-6 (2002).
25. Milenkovic, L., Scott, M. P. & Rohatgi, R. Lateral transport of Smoothened from the plasma membrane to the membrane of the cilium. J Cell Biol 187, 365-74 (2009).
26. Wilson, C. W., Chen, M.-H. & Chuang, P.-T. Smoothened adopts multiple active and inactive conformations capable of trafficking to the primary cilium. PLoS ONE 4, e5182 (2009).
27. Schuller, U. et al. Acquisition of granule neuron precursor identity is a critical determinant of progenitor cell competence to form Shh-induced medulloblastoma. Cancer Cell 14, 123-134 (2008).
28. Lauth, M., Bergström, A., Shimokawa, T. & Toftgård, R Inhibition of GLI-mediated transcription and tumor cell growth by small-molecule antagonists. Proc Natl Acad Sci USA 104, 8455-60 (2007).
29. Sinha, S. & Chen, J. K. Purmorphamine activates the Hedgehog pathway by targeting Smoothened. Nat. Chem Biol 2, 29-30 (2006).
30. Rohatgi, R., Milenkovic, L., Corcoran, R. & Scott, M. Hedgehog signal transduction by Smoothened: Pharmacologic evidence for a 2-step activation process. Proc Natl Acad Sci USA (2009).
31. Johnson, M. Development of fluticasone propionate and comparison with other inhaled corticosteroids. J Allergy Clin Immunol 101, S434-9 (1998).
32. Heine, V. M. & Rowitch, D. H. Hedgehog signaling has a protective effect in glucocorticoid-induced mouse neonatal brain injury through an 11betaHSD2-dependent mechanism. J Clin Invest 119, 267-77 (2009).
33. Shaw, A., Gipp, J. & Bushman, W. The Sonic Hedgehog pathway stimulates prostate tumor growth by paracrine signaling and recapitulates embryonic gene expression in tumor myofibroblasts. Oncogene (2009).
34. Mantovani, A., Allavena, P., Sica, A. & Balkwill, F. Cancer-related inflammation. Nature 454, 436-444 (2008).
35. Sui, M. H., Chen, F., Chen, Z. & Fan, W. M. Glucocorticoids interfere with therapeutic efficacy of paclitaxel against human breast and ovarian xenograft tumors. International Journal of Cancer 119, 712-717 (2006).
36. Zhang, C. et al. Dexamethasone desensitizes hepatocellular and colorectal tumours toward cytotoxic therapy. Cancer Letters 242, 104-111 (2006).
37. Herr, I. et al. Glucocorticoid cotreatment induces apoptosis resistance toward cancer therapy in carcinomas. Cancer Research 63, 3112-3120 (2003).
38. Zhang, C. W. et al. Glucocorticoid-mediated inhibition of chemotherapy in ovarian carcinomas. International Journal of Oncology 28, 551-558 (2006).
39. Zhang, C. W. et al. Corticosteroid co-treatment induces resistance to chemotherapy in surgical resections, xenografts and established cell lines of pancreatic cancer. Bmc Cancer 6,-(2006).
40. Sherlock, P. & Hartmann, W. H. Adrenal Steroids and Pattern of Metastases of Breast Cancer. Jama-Journal of the American Medical Association 181, 313-& (1962).
41. Fukao, K. et al. Effects of Fluocinolone Acetonide on Mouse Skin Sterol-Metabolism and 2-Stage Carcinogenesis. Carcinogenesis 9, 1661-1664 (1988).
42. Brady, M. E., Sartiano, G. P., Rosenblum, S. L., Zaglama, N. E. & Bauguess, C. T. The Pharmacokinetics of Single High-Doses of Dexamethasone in Cancer-Patients. European Journal of Clinical Pharmacology 32, 593-596 (1987).
43. Taipale, J. et al. Effects of oncogenic mutations in Smoothened and Patched can be reversed by cyclopamine. Nature 406, 1005-9 (2000).
44. Chan, J. A. et al. Proteoglycan interactions with Sonic Hedgehog specify mitogenic responses. Nature Neuroscience 12, 409-417 (2009).

All patents and other publications identified in the specification and examples are expressly incorporated herein by reference for all purposes. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

What is claimed is:

1. A method for modulating a Hedgehog signaling pathway in a cell, the method comprising contacting a cell with a glucocorticoid, or a compound of formula (I): 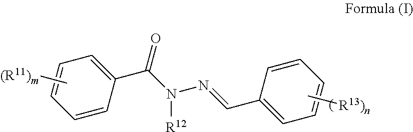
wherein:
R¹¹ is independently for each occurrence optionally substituted alkyl, optionally substituted alkenyl, optionally substituted alkynyl, optionally substituted cyclyl, optionally substituted heterocyclyl, optionally substituted aryl, optionally substituted heteroaryl, OR¹⁴, NO₂, CN, CF₃, halo, C(O)R¹⁴, CO₂R¹⁴, SOR¹⁴, SO₂R¹⁴, or N(R¹⁵);
R¹² is H, optionally substituted alkyl, optionally substituted alkenyl, optionally substituted alkynyl, optionally substituted cyclyl, optionally substituted heterocyclyl, optionally substituted aryl, optionally substituted heteroaryl, OR¹⁴, C(O)R¹⁴, or CO₂R¹⁴;
R¹³ is independently for each occurrence optionally substituted alkyl, optionally substituted alkenyl, optionally substituted alkynyl, optionally substituted cyclyl, optionally substituted heterocyclyl, optionally substituted aryl, optionally substituted heteroaryl, OR¹⁴, NO₂, CN, CF₃, halo, C(O)R¹⁴, CO₂R¹⁴, SOR¹⁴, SO₂R¹⁴, or N(R¹⁵);
R¹⁴ is independently for each occurrence optionally substituted alkyl, optionally substituted alkenyl, optionally substituted alkynyl, optionally substituted cyclyl, optionally substituted heterocyclyl, optionally substituted aryl, or optionally substituted heteroaryl;
R¹⁵ is independently for each occurrence H, optionally substituted alkyl, optionally substituted alkenyl, optionally substituted alkynyl, optionally substituted cyclyl, optionally substituted heterocyclyl, optionally substituted aryl, optionally substituted heteroaryl, C(O)R¹⁴, or CO₂R¹⁴;
m is 0, 1, 2, 3, 4, or 5;
n is 0, 1, 2, 3, 4, or 5; and
isomers and pharmaceutically acceptable salts thereof,
or a compound of formula (II): 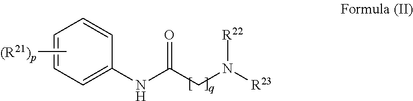
wherein:
R²¹ is independently for each occurrence optionally substituted alkyl, optionally substituted alkenyl, optionally substituted alkynyl, optionally substituted cyclyl, optionally substituted heterocyclyl, optionally substituted aryl, optionally substituted heteroaryl, OR²⁴, NO₂, CN, CF₃, halo, OC(O)R²⁴, C(O)R²⁴, CO₂R²⁴, C(O)N(R²⁴)₂, SOR²⁴, SO₂R²⁴, N(R²⁴)₂, NHC(O)R²⁴, NHCO₂R²⁴, or two R²¹s together with the carbons they are attached to form an optionally substituted 5-8 member cyclyl or an optionally substituted 5-8 member heterocyclyl;
R²² and R²³ are independently H, optionally substituted alkyl, optionally substituted alkenyl, optionally substituted alkynyl, optionally substituted cyclyl, optionally substituted heterocyclyl, optionally substituted aryl, optionally substituted heteroaryl, optionally substituted —(CH₂)ₜ—R²⁴, or R²² and R²³ together with the nitrogen they are attached to form an optionally substituted 5-8 membered ring;
R²⁴ is independently for each occurrence optionally substituted alkyl, optionally substituted alkenyl, optionally substituted alkynyl, optionally substituted cyclyl, optionally substituted heterocyclyl, optionally substituted aryl, or optionally substituted heteroaryl;
p is 0, 1, 2, 3, 4, or 5;
q is 1, 2, 3, 4, 5, or 6;
t is 1, 2, 3, 4, 5, or 6; and
isomers and pharmaceutically acceptable salts thereof,
and any combinations thereof.

2. The method of claim 1, wherein the compound inhibits the Hedgehog signaling pathway.

3. The method of claim 1, wherein the compound activates the Hedgehog signaling pathway.

4. The method of claim 1, wherein the compound inhibits the translocation of Smoothened (Smo) to the primary cilium and/or the accumulation of Smo in the primary cilium.

5. The method of claim 1, wherein the compound increases the translocation of Smoothened (Smo) to the primary cilium and/or the accumulation of Smo in the primary cilium.

6. The method of claim 1, wherein said Hedgehog signaling is sonic Hedgehog signaling, desert Hedgehog signaling, or Indian Hedgehog signaling.

7. The method of claim 1, wherein the cell has a phenotype of Ptch1 loss-of-function, Hedgehog gain-of-function, smoothened gain-of-function, Gli gain-of-function or over expression of Hedgehog ligands.

8. The method of claim 1, wherein the cell is a cancer cell.

9. The method of claim 1, wherein the compound of formula (I) is

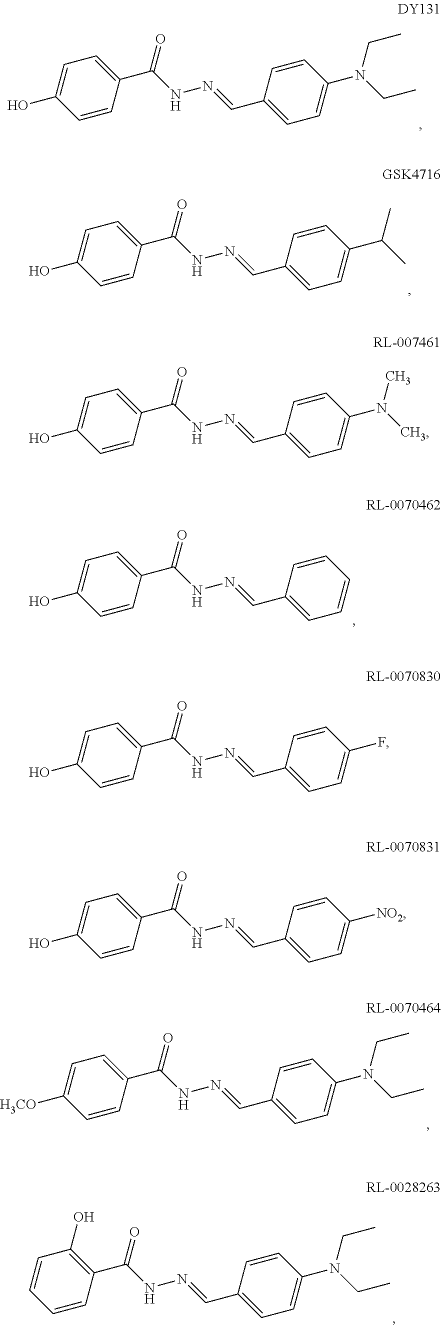

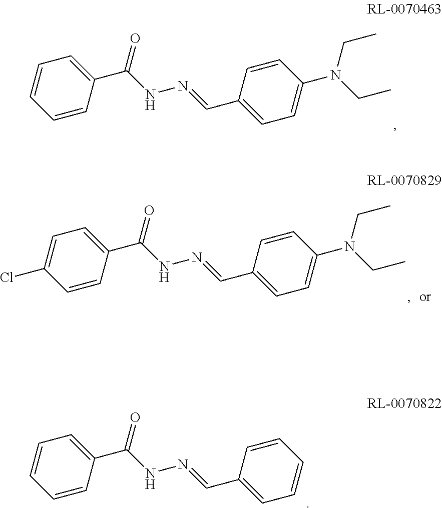 [SMILES: CCN(CC)c1ccc(/C=N/NC(=O)c2ccccc2)cc1 ; CCN(CC)c1ccc(/C=N/NC(=O)c2ccc(Cl)cc2)cc1 ; O=C(N/N=C/c1ccccc1)c1ccccc1]

10. The method of claim 1, wherein the compound of formula (II) is selected from those shown in FIGS. 24-36.

11. The method of claim 1, wherein the contact is in vitro.

12. The method of claim 1, wherein the contact is in vivo.

13. The method of claim 12, wherein in vivo contact is in a mammal.

14. The method of claim 12, wherein said in vivo contact is in a mouse or a rat.

15. The method of claim 12, wherein said in vivo contact is in a human.

16. The method of claim 12, wherein said in vivo contact is in a subject, which subject is afflicted with cancer, a precancerous condition and/or metastasis.

17. The method of claim 16, wherein cancer is selected from the group consisting of breast cancer, biliary tract cancer, bladder cancer, brain cancer including Glioblastomas and medulloblastomas; cervical cancer; choriocarcinoma; colon cancer; endometrial cancer; esophageal cancer, gastric cancer; hematological neoplasms including acute lymphocytic and myelogenous leukemia; T-cell acute lymphoblastic leukemia/lymphoma; hairy cell leukemia; chronic myelogenous leukemia, multiple myeloma; AIDS-associated leukemias and adult T-cell leukemia lymphoma; intraepithelial neoplasms including Bowen's disease and Paget's disease; liver cancer; lung cancer; lymphomas including Hodgkin's disease and lymphocytic lymphomas; neuroblastomas; oral cancer including squamous cell carcinoma; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, and osteosarcoma; skin cancer including melanoma, Merkel cell carcinoma, Kaposi's sarcoma, basal cell carcinoma, and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; and renal cancer including adenocarcinoma and Wilms tumor.

18. A method for modulating differentiated state, survival, and/or proliferation of a cell, the method comprising contacting the cell with a glucocorticoid, or a compound of formula (I): 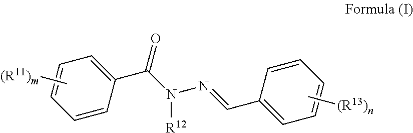
wherein:
R¹¹ is independently for each occurrence optionally substituted alkyl, optionally substituted alkenyl, optionally substituted alkynyl, optionally substituted cyclyl, optionally substituted heterocyclyl, optionally substituted aryl, optionally substituted heteroaryl, OR¹⁴, NO₂, CN, CF₃, halo, C(O)R¹⁴, CO₂R¹⁴, SOR¹⁴, SO₂R¹⁴, or N(R¹⁵);
R¹² is H, optionally substituted alkyl, optionally substituted alkenyl, optionally substituted alkynyl, optionally substituted cyclyl, optionally substituted heterocyclyl, optionally substituted aryl, optionally substituted heteroaryl, OR¹⁴, C(O)R¹⁴, or CO₂R¹⁴;
R¹³ is independently for each occurrence optionally substituted alkyl, optionally substituted alkenyl, optionally substituted alkynyl, optionally substituted cyclyl, optionally substituted heterocyclyl, optionally substituted aryl, optionally substituted heteroaryl, OR¹⁴, NO₂, CN, CF₃, halo, C(O)R¹⁴, CO₂R¹⁴, SOR¹⁴, SO₂R¹⁴, or N(R¹⁵);
R¹⁴ is independently for each occurrence optionally substituted alkyl, optionally substituted alkenyl, optionally substituted alkynyl, optionally substituted cyclyl, optionally substituted heterocyclyl, optionally substituted aryl, or optionally substituted heteroaryl;
R¹⁵ is independently for each occurrence H, optionally substituted alkyl, optionally substituted alkenyl, optionally substituted alkynyl, optionally substituted cyclyl, optionally substituted heterocyclyl, optionally substituted aryl, optionally substituted heteroaryl, C(O)R¹⁴, or CO₂R¹⁴;
m is 0, 1, 2, 3, 4, or 5;
n is 0, 1, 2, 3, 4, or 5; and
isomers and pharmaceutically acceptable salts thereof,
or a compound of formula (II): 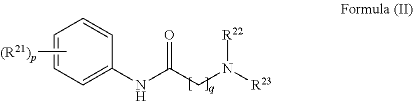
wherein:
R²¹ is independently for each occurrence optionally substituted alkyl, optionally substituted alkenyl, optionally substituted alkynyl, optionally substituted cyclyl, optionally substituted heterocyclyl, optionally substituted aryl, optionally substituted heteroaryl, OR²⁴, NO₂, CN, CF₃, halo, OC(O)R²⁴, C(O)R²⁴, CO₂R²⁴, C(O)N(R²⁴)₂, SOR²⁴, SO₂R²⁴, N(R²⁴)₂, NHC(O)R²⁴, NHCO₂R²⁴, or two R²¹s together with the carbons they are attached to form an optionally substituted 5-8 member cyclyl or an optionally substituted 5-8 member heterocyclyl;
R²² and R²³ are independently H, optionally substituted alkyl, optionally substituted alkenyl, optionally substituted alkynyl, optionally substituted cyclyl, optionally substituted heterocyclyl, optionally substituted aryl, optionally substituted heteroaryl, optionally substituted —(CH₂)ₜ—R²⁴, or R²² and R²³ together with the nitrogen they are attached to form an optionally substituted 5-8 membered ring;
R²⁴ is independently for each occurrence optionally substituted alkyl, optionally substituted alkenyl, optionally substituted alkynyl, optionally substituted cyclyl, optionally substituted heterocyclyl, optionally substituted aryl, or optionally substituted heteroaryl;
p is 0, 1, 2, 3, 4, or 5;
q is 1, 2, 3, 4, 5, or 6;
t is 1, 2, 3, 4, 5, or 6; and
isomers and pharmaceutically acceptable salts thereof,
and any combinations thereof.

19. A method for treating or preventing cancer, a precancerous condition and/or metastasis, the method comprising administering to a subject in need thereof a therapeutically effective amount of a compound of formula (I): 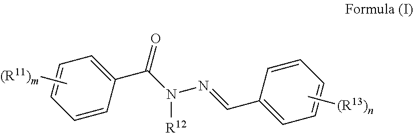
wherein:
R¹¹ is independently for each occurrence optionally substituted alkyl, optionally substituted alkenyl, optionally substituted alkynyl, optionally substituted cyclyl, optionally substituted heterocyclyl, optionally substituted aryl, optionally substituted heteroaryl, OR¹⁴, NO₂, CN, CF₃, halo, C(O)R¹⁴, CO₂R¹⁴, SOR¹⁴, SO₂R¹⁴, or N(R¹⁵);
R¹² is H, optionally substituted alkyl, optionally substituted alkenyl, optionally substituted alkynyl, optionally substituted cyclyl, optionally substituted heterocyclyl, optionally substituted aryl, optionally substituted heteroaryl, OR¹⁴, C(O)R¹⁴, or CO₂R¹⁴;
R¹³ is independently for each occurrence optionally substituted alkyl, optionally substituted alkenyl, optionally substituted alkynyl, optionally substituted cyclyl, optionally substituted heterocyclyl, optionally substituted aryl, optionally substituted heteroaryl, OR¹⁴, NO₂, CN, CF₃, halo, C(O)R¹⁴, CO₂R¹⁴, SOR¹⁴, SO₂R¹⁴, or N(R¹⁵);
R¹⁴ is independently for each occurrence optionally substituted alkyl, optionally substituted alkenyl, optionally substituted alkynyl, optionally substituted cyclyl, optionally substituted heterocyclyl, optionally substituted aryl, or optionally substituted heteroaryl;
R¹⁵ is independently for each occurrence H, optionally substituted alkyl, optionally substituted alkenyl, optionally substituted alkynyl, optionally substituted cyclyl, optionally substituted heterocyclyl, optionally substituted aryl, optionally substituted heteroaryl, C(O)R¹⁴, or CO₂R¹⁴;
m is 0, 1, 2, 3, 4, or 5;
n is 0, 1, 2, 3, 4, or 5; and
isomers and pharmaceutically acceptable salts thereof,
or a compound of formula (II): 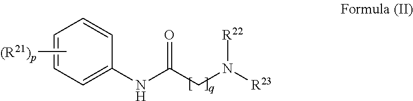
wherein:
R²¹ is independently for each occurrence optionally substituted alkyl, optionally substituted alkenyl, optionally substituted alkynyl, optionally substituted cyclyl, optionally substituted heterocyclyl, optionally substituted aryl, optionally substituted heteroaryl, OR²⁴, NO₂, CN, CF₃, halo, OC(O)R²⁴, C(O)R²⁴, CO₂R²⁴, C(O)N(R²⁴)₂, SOR²⁴, SO₂R²⁴, N(R²⁴)₂, NHC(O)R²⁴, NHCO₂R²⁴, or two R²¹s together with the carbons they are attached to form an optionally substituted 5-8 member cyclyl or an optionally substituted 5-8 member heterocyclyl;
R²² and R²³ are independently H, optionally substituted alkyl, optionally substituted alkenyl, optionally substituted alkynyl, optionally substituted cyclyl, optionally substituted heterocyclyl, optionally substituted aryl, optionally substituted heteroaryl, optionally substituted —(CH₂)ᵣR²⁴, or R²² and R²³ together with the nitrogen they are attached to form an optionally substituted 5-8 membered ring;
R²⁴ is independently for each occurrence optionally substituted alkyl, optionally substituted alkenyl, optionally substituted alkynyl, optionally substituted cyclyl, optionally substituted heterocyclyl, optionally substituted aryl, or optionally substituted heteroaryl;
p is 0, 1, 2, 3, 4, or 5;
q is 1, 2, 3, 4, 5, or 6;
t is 1, 2, 3, 4, 5, or 6; and
isomers and pharmaceutically acceptable salts thereof,
and any combinations thereof.
